(19) United States
(12) Patent Application Publication
(10) Pub. No.: US 2005/0020559 A1
(43) Pub. Date: Jan. 27, 2005

(54) Chlorin photosensitizing agents for use in photodynamic therapy

(76) Inventors: Byron C Robinson, Santa Barbara, CA (US); Avinash Phadke, Branford, CT (US)

Correspondence Address: FINNEGAN, HENDERSON, FARABOW, GARRETT & DUNNER;LLP, 1300 I STREET, NW, WASHINGTON, DC, 20005, US

(21) Appl. No.: 10/491,328

(22) PCT Filed: Oct. 2, 2002
(86) PCT No.: PCT/US02/29833

Related U.S. Application Data
(60) Provisional application No. 60/326,450, filed on Oct. 3, 2001.

(51) Int. Cl. C07D 487/22; A61K 31/555

(52) U.S. Cl. 514/185; 514/410; 540/145

(57) ABSTRACT

Chlorin compounds and compositions useful in photodynamic therapy for treating ophtalmic, cardiovascular, skin, and cancer or malignant diseases.

### FIELD OF THE INVENTION

This invention relates to a class of free base and metallated chlorin compounds having phototherapeutic properties utilizable in photodynamic therapy for the treatment of diseases applicable to photodynamic therapy, in particular, ophthalmic diseases.

### BACKGROUND OF THE INVENTION

Photodynamic therapy (“PDT”) is a new modality for the treatment of malignancies, diseased tissue, hyperproliferating tissues, normal tissues or pathogens. PDT involves a localized or systemic administration of a photosensitizing compound followed by exposure of target tissue to photoactivating light. The photoactivating light excites the photosensitizer which, in turn, interacts with singlet oxygen causing the production of cytotoxic oxygen species. The interaction of the cytotoxic oxygen species with tissues in which the photosensitizer is localized results in a modification of the tissue, resulting in a desired clinical effect. The tissue specificity of the resultant phototoxic damage is determined largely (although not entirely) by the relative concentrations of the photosensitizer in each tissue at the time of exposure to the photoactivating light. The method of light delivery is also an important therapeutic factor.

Following systemic administration, many photosensitizers accumulate to varying degrees within tissues depending on the pharmacokinetic and distribution profile of the photosensitizing compound and the cell types comprising the tissues. The chemical factors that enable certain photosensitizers to accumulate to a greater degree at a target site than other photosensitizers is not well understood. In addition, the biological factors that result in the preferential uptake of some photosensitizers in certain tissue types compared to others is not well understood either. It is very clear, however, that each photosensitizer has its own distribution and pharmacokinetic properties within different tissues and these properties determine the relative usefulness of the drug for the therapy. Currently, only rigorous screening and biological evaluation in appropriate model systems identifies suitable photosensitizers that display the characteristics necessary within the diseased or target tissues for an effective therapy.

An important visible clinical role for photodynamic therapy is in the treatment of choroidal neovascularization (CNV) of the eye. CNV is a pathologic feature of many eye diseases that leads to severe visual loss. The etiology of the disease is such that new abnormal blood vessels proliferate from the choriocapillaris through defects in Bruch's membrane under the retinal pigment epithelium (RPE), forming vascular membranes. These new vessels leak serous fluid that may give rise to serous and hemorrhagic detachment of the RPE and neurosensory retina and stimulate fibrous disciform scarring with subsequent vision loss. In age related macular degeneration (ARMD), the abnormal leaking neovessel growth occurs in the area of the macula of the eye. Leakage of serous fluid and damage to the RPE leads to rapid and often debilitating vision loss. The treatment of ARMD using photodynamic therapy and a drug called Visudyne® (QLT Inc., Vancouver Canada) has demonstrated modest, but statistically significant 2 year stabilization of patients' vision. While statistically significant, the treatment produces results that are only 16% better than that observed in the placebo group. It is clear that other photosensitizers that display greater efficacy in animal models and humans are desired for effective treatment of ARMD. Other ophthalmic disease applications that may be applicable to treatment with photodynamic therapy include proliferative retinopathies, macular edema, corneal neovascularization, conjunctival neovascularization, ocular tumors, viral retinitis, adjunct to glaucoma filtration surgery and cyclodestruction, posterior capsule opacification and dry ARMD.

### SUMMARY OF THE INVENTION

To achieve these and other advantages, and in accordance with the purpose of the invention, as embodied and broadly described herein, the present invention, in one aspect, provides phototherapeutic compositions of derivatized free base and metallo-chlorin compounds of formula I, which may be used in a medicament for treatment of ophthalmic diseases or other disease indications:

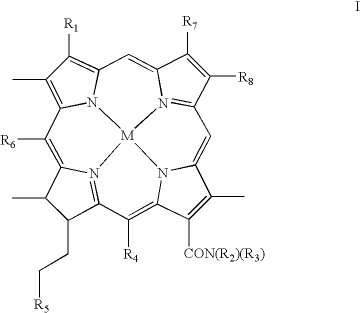

In formula I:
R₁, R₅, R₆, R₇, and R₈ can be the same or different and are selected from the group consisting of:
   H, halogen, methyl, ethyl, substituted or unsubstituted C1-C20 alkyl, heteroalkyl, haloalkyl, heterohaloalkyl, cyclic alkyl, aryl, substituted aryl, alkenyl, haloalkenyl, substituted alkenyl, alkynyl, substituted alkynyl, amide, ester, ethers, polyethers, alkoxy group, aryloxy group, haloalkoxy group, amino group, alkylcarbonyloxy group, alkoxycarbonyl group, aryloxycarbonyl group, azo group, arylcarbonyloxy group, alkoxycarbonyloxy group, aryloxycarbonyloxy group, sulfinyl group, sulfonyl group, silil group, carbamoyl group, heterocyclic group, nitro group, nitroso group, formyloxy group, isocyano group, cyanate group, isocyanate group, thiocyanate group, isothiocyanate group, N(alkyl)₂, N(aryl)₂, CH═CH(aryl), CH═CHCH₂N(CH₃)₂, CH═CHCH₂N⁺(CH₃)₃A, CH═N(alkyl)₂⁺A or N(alkyl)₃⁺A, CN, OH, CHO, COCH₃, CO(alkyl), CO₂H, CO₂Na, CO₂K, CH(CH₃)OH, CH(CH₃)O-alkyl, CH(CH₃)O-alkoxy, CH(CH₃)O-aryl, CH(CH₃)NH-alkyl, CH(CH₃)NH-cycloalkyl, CH(CH₃)NH-heteroalkyl, CH(CH₃)NH-heteroalkoxy, CH(CH₃)-(amino acid), CH(CH₃)-(amino acid ester), CH(CH₃)-(amino acid amide), C(X)₂C(X)₃ (where X is H or halogen), CH═NR₉ (where R₉ is OH, O-alkyl, O-ether, or O-alkylamino), NHCOCH₂N(CH₃)₂, NHCOCH₂N(CH₃)₃⁺A, NHCOCH₂-(pyridinium)⁺A, (CH₂)ₙO-alkoxy, or (CH₂)ₙO-alkyl, where n is an integer ranging from 0 to 8 and A is a charge balancing ion;
   CO₂R₁₀, where R₁₀ is selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons;
   (CH₂)ₙOH, or (CH₂)ₙOR₁₁, where R₁₁ is selected from alkyl, haloalkyl, heteroalkyl, haloheteroalkyl, aryl, heteroaryl, heterocycle, a protecting group, a mono-, di- or polyhydroxyalkyl residue, a mono-, di-, or polyhydroxyaryl residue, or a functional group of less than about 100,000 daltons, and n is an integer ranging from 0 to 4;
   (CH₂)ₙCO₂R₁₂, (CHX)ₙCO₂R₁₂, or (CX₂)ₙCO₂R₁₂, where X is selected from OH, OR₁₃, or a halogen, and R₁₂ and R₁₃ can be the same or different and are selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons, and n is an integer ranging from 1 to 4;
   CONH(R₁₄), CONHNH(R₁₄), CO(R₁₄), CON(R₁₄)₂, CON(R₁₄)(R₁₅) (CH₂)ₙCONH(R₁₄), (CH₂)ₙCON(R₁₄)₂, (CH₂)ₙCOR₁₄, (CH₂)ₙCON(R₁₄)(R₁₅), (CX₂)ₙCONH(R₁₄), (CX₂)ₙCON(R₁₄)₂, (CX₂)ₙCON(R₁₄)(R₁₅), (CX₂)ₙCOR₁₄, (CH₂)ₙCONHNH(R₁₄), (CX₂)ₙCONHNH(R₁₄), (CHX)ₙCONH(R₁₅), (CHX)ₙCONHNH(R₁₄), (CHX)ₙCO(R₁₄), (CHX)ₙCON(R₁₄)₂, or (CHX)ₙCON(R₁₄)(R₁₅), where X is selected from OH, OR₁₆, or a halogen, and R₁₄, R₁₅ and R₁₆ can be the same or different and are selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, or a functional group of less than about 100,000 daltons, and n is an integer ranging from 1 to 4;
   S(R₁₇), CH(CH₃)S(R₁₇), (CH₂)ₙS(R₁₇), (CH₂)ₙNH(R₁₇), (CH₂)ₙNHNH(R₁₇), (CH₂)ₙN(R₁₇)₂, (CH₂)ₙN(R₁₇)(R₁₈), or (CH₂)ₙN(R₁₇)(R₁₈)(R₁₉)⁺A, CH═N(R₁₇), or CH═NN(R₁₇)(R₁₈), where R₁₇, R₁₈ and R₁₉ can be the same or different and are selected from H, OH, O-alkyl, NH₂, acetyl, a straight or branched chain C1-C20 alkyl, haloalkyl, heteroalkyl, haloheteroalkyl, aryl, heteroaryl, heterocycle, amino acids (provided —NH(R₁₇) or —N(R₁₇)(R₁₈) is part of the amino acid), a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, where R₁₇, R₁₈ and R₁₉ may together possess the atoms necessary to constitute an aromatic ring system, n is an integer ranging from 0 to 4, and A is a physiologically acceptable counter ion;
   (CH₂)ₙOPO(OR₂₀)₂ or (CH₂)ₙPO(OR₂₀)₂, where R₂₀ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, and n is an integer ranging from 0 to 4;
   (CH₂)ₙNHCOR₂₁, or (CH₂)ₙNHNHCOR₂₁, where R₂₁ is selected from a straight or branched chain C1-C20 alkyl, haloalkyl, heteroalkyl, haloheteroalkyl, aryl, heteroaryl, heterocycle, or a functional group of less than about 100,000 daltons, and n is an integer ranging from 0 to 4;
   SO₃R₂₂, SO₂NHR₂₂, SO₂N(R₂₂)₂, SO₂NHNHR₂₂, SO₂R₂₂, (CH₂)ₙSO₂NHR₂₂, (CH₂)ₙSO₂N(R₂₂)₂, (CH₂)ₙSO₂NHNHR₂₂, or (CH₂)ₙSO₂R₂₂, where R₂₂ is selected from H, OH, A⁺ (where A⁺ is a physiologically acceptable counter ion), a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, and NHR₂₂ can also be an amino acid, an amino acid salt, an amino acid ester residue, an amino acid amide residue, and n is an integer ranging from 0 to 4;
   aryl or substituted aryl, which may optionally bear one or more substituents with a molecular weight of less than or equal to about 100,000 daltons;
   R₂ and R₃ can be the same or different and are selected from H, C1-C20 alkyl, C1-C20 cyclic alkyl, aryl, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN((CH₂)ₘN(CH₃)₂)₂, (CH₂)ₙO-haloalkyl, (CH₂)ₙN((CH₂)ₘN(CH₃)₃⁺A)₂, ((CH₂)ₙO)ₘ((CH₂)_{Q}O)COCH₃, a mono-, di-, or polyhydroxyalkyl residue, a mono-, di-, or polyhydroxyaryl residue, a mono-, di-, or polyetheralkyl residue, a mono-, di-, or polyetheraryl residue, and Q, n and m are integers ranging from 0 to 10,000, and A is a physiologically acceptable counter ion;
   R₄ is selected from:
   (CH₂)ₙCO₂R₂₃, (CHX)ₙCO₂R₂₃, or (CX₂)ₙCO₂R₂₃, where X is selected from OH, OR₂₄, SR₂₄, or a halogen and R₂₃ and R₂₄ can be the same or different and are selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons, and n is an integer ranging from 1 to 4;
   (CH₂)ₙCONH(R₂₅), (CH₂)ₙCON(R₂₅)₂, (CH₂)ₙCOR₂₅, (CH₂)ₙCON(R₂₅)(R₂₆), (CX₂)ₙCONH(R₂₅), (CX₂)ₙCON(R₂₅)₂, (CX₂)ₙCON(R₂₅)(R₂₆), (CX₂)ₙCOR₂₅, (CH₂)ₙCONHNH(R₂₅), (CX₂)ₙCONHNH(R₂₅), (CHX)ₙCONH(R₂₅), (CHX)ₙCONHNH(R₂₅), (CHX)ₙCO(R₂₅), (CHX)ₙCON(R₂₅)₂, or (CHX)ₙCON(R₂₅)(R₂₆), where X is selected from OH, OR₂₇, SR₂₇, or a halogen, and R₂₅, R₂₆ and R₂₇ can be the same or different and are selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, or a functional group of less than about 100,000 daltons, and n is an integer ranging from 1 to 4;
   (CH₂)ₙS(R₂₈), (CH₂)ₙNH(R₂₈), (CH₂)ₙNHNH(R₂₈), (CH₂)ₙN(R₂₈)₂, (CH₂)ₙN(R₂₈)(R₂₉), or (CH₂)ₙN(R₂₈)(R₂₉)(R₃₀)⁺A, where R₂₈, R₂₉ and R₃₀ can be the same or different and are selected from H, OH, NH₂, acetyl, a straight or branched chain C1-C20 alkyl, haloalkyl, heteroalkyl, haloheteroalkyl, aryl, heteroaryl, heterocycle, amino acids (provided —NH(R₂₈) is part of the amino acid), amino acid esters (provided —NH(R₂₈) is part of the amino acid ester), amino acid amides (provided —NH(R₂₈) is part of the amino acid amide), a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, where R₂₈, R₂₉ and R₃₀ together may possess the atoms necessary to constitute an aromatic ring system, n is an integer ranging from 1 to 4, and A is a physiologically acceptable counter ion;
   (CH₂)ₙOPO(OR₃₁)₂, or (CH₂)ₙPO(OR₃₁)₂, where R₃₁ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, and n is an integer ranging from 0 to 4;
   (CH₂)ₙOH or (CH₂)ₙOR₃₂, where R₃₂ is selected from alkyl, haloalkyl, heteroalkyl, haloheteroalkyl, aryl, heteroaryl, heterocycle, a protecting group, a mono-, di- or polyhydroxyalkyl residue, a mono-, di-, or polyhydroxyaryl residue, or a functional group of less than about 100,000 daltons, and n is an integer ranging from 0 to 4;
   (CH₂)ₙNHCOR₃₃ or (CH₂)ₙNHNHCOR₃₃, where R₃₃ is selected from a straight or branched chain C1-C20 alkyl, OH, haloalkyl, heteroalkyl, haloheteroalkyl, aryl, heteroaryl, heterocycle, or a functional group of less than about 100,000 daltons, and n is an integer ranging from 1 to 4;
   (CH₂)ₙSO₂NHR₃₄, (CH₂)ₙSO₂N(R₃₄)₂, (CH₂)ₙSO₂NHNHR₃₄, (CH₂)ₙSO₂R₃₄ (CH₂)ₙOSO₂NHR₃₄, (CH₂)ₙOSO₂N(R₃₄)₂, (CH₂)ₙOSO₂NHNHR₃₄, or (CH₂)ₙOSO₃R₃₄, where R₃₄ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, NHR₃₄ can be an amino acid, an amino acid salt, an amino acid ester residue, an amino acid amide residue, and n is an integer ranging from 1 to 4; and
   aryl or substituted aryl, which may optionally bear one or more substituents with a molecular weight of less than or equal to about 100,000 daltons.

In formula I, M can be selected from 2H, a metal cation, and photoactive metal ions being preferably selected from Ga³⁺, Pt²⁺, Pd²⁺, Sn⁴⁺, In³⁺, Ge⁴⁺, Si⁴⁺, Al³⁺, Zn²⁺, Mg²⁺, wherein optionally associated with the metal ion is the appropriate number of physiologically acceptable charge balancing counter ions.

In a preferred embodiment of formula I, when R₁ is vinyl, R₂ is H, R₄ is CH₂CO₂CH₃, R₅ is CO₂H, R₆ is H, R₇ is CH₃, and R₈ is CH₂CH₃, R₃ cannot be (CH₂)₂CH₃, CH₂CH₃, CH(CH₃)₂, (CH₂)₃CH₃, CH₂-phenyl, or cyclohexyl.

In accordance with the invention, another preferred class of chlorins that may be used in a medicament for treatment of ophthalmic diseases or other disease indications is shown below:

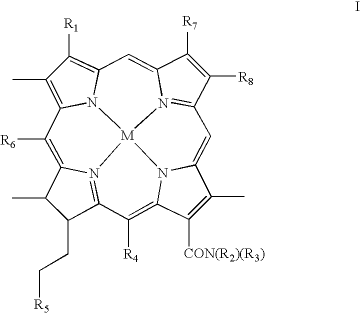

In this preferred class of chlorines within formula I:
R₁, R₇, and R₈ can be the same or different and are selected from vinyl, hydroxyalkyl, alkylether, and CH₂CH₂OCOCH₃;
R₆ is selected from methyl, halogen, CH═CHCHO, CH═CHCH₂OH, CH═CHCH₂O-alkyl, and CH═CHCH₂O-alkylether;
R₄ is selected from:
   (CH₂)ₙCO₂R₉, (CHX)ₙCO₂R₉, or (CX₂)ₙCO₂R₉, where X is selected from OH, OR₁₀, SR₁₀, or a halogen, and R₉ and R₁₀ can be the same or different and are selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons, and n is an integer ranging from 1 to 4;
   (CH₂)ₙCONH(R₁₁), (CH₂)ₙCON(R₁₁)₂, (CH₂)ₙCOR₁₁, (CH₂)ₙCON(R₁₁)(R₁₂), (CX₂)ₙCONH(R₁₁), (CX₂)ₙCON(R₁₂)₂, (CX₂)ₙCON(R₁₁)(R₁₂), (CX₂)ₙCOR₁₁, (CH₂)ₙCONHNH(R₁₁), (CX₂)ₙCONHNH(R₁₁), (CHX)ₙCONH(R₁₁), (CHX)ₙCONHNH(R₁₁), (CHX)ₙCO(R₁₁), (CHX)ₙCON(R₁₁)₂, or (CHX)ₙCON(R₁₁)(R₁₂), where X is selected from OH, OR₁₃, SR₁₃, or a halogen, and R₁₁, R₁₂ and R₁₃ can be the same or different and are selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, or a functional group of less than about 100,000 daltons, and n is an integer ranging from 1 to 4;
   (CH₂)ₙS(R₁₄), (CH₂)ₙNH(R₁₄), (CH₂)ₙNHNH(R₁₄), (CH₂)ₙN(R₁₄)₂, (CH₂)ₙN(R₁₄)(R₁₅), or (CH₂)ₙN(R₁₄)(R₁₅)(R₁₆)⁺A, where R₁₄, R₁₅ and R₁₆ can be the same or different and are selected from H, OH, NH₂, acetyl, a straight or branched chain C1-C20 alkyl, haloalkyl, heteroalkyl, haloheteroalkyl, aryl, heteroaryl, heterocycle, amino acids (provided —NH(R₁₄) is part of the amino acid), amino acid esters (provided —NH(R₁₄) is part of the amino acid ester), amino acid amides (provided —NH(R₁₄) is part of the amino acid amide), a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, where R₁₄, R₁₅ and R₁₆ together may possess the atoms necessary to constitute an aromatic ring system, n is an integer ranging from 1 to 4, and A is a physiologically acceptable counter ion;
   (CH₂)ₙOPO(OR₁₇)₂, or (CH₂)ₙPO(OR₁₇)₂, where R₁₇ is selected from H, OH, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, and n is an integer ranging from 0 to 4;
   (CH₂)ₙNHCOR₁₈, or (CH₂)ₙNHNHCOR₁₈, where R₁₈ is selected from a straight or branched chain C1-C20 alkyl, haloalkyl, heteroalkyl, haloheteroalkyl, aryl, heteroaryl, heterocycle, or a functional group of less than about 100,000 daltons, and n is an integer ranging from 0 to 4;
   (CH₂)ₙSO₂NHR₁₉, (CH₂)ₙSO₂N(R₁₉)₂, (CH₂)ₙSO₂NHNHR₁₉, (CH₂)ₙSO₂R₁₉, (CH₂)ₙOSO₂NHR₁₉, (CH₂)ₙOSO₂N(R₁₉)₂, (CH₂)ₙOSO₂NHNHR₁₉, or (CH₂)ₙOSO₃R₁₉, where R₁₉ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, and NHR₁₉ can also be an amino acid, an amino acid salt, an amino acid ester residue, an amino acid amide residue, and n is an integer between 0 and 4;
   (CH₂)ₙOH, or (CH₂)ₙOR₂₀, where R₂₀ is selected from alkyl, haloalkyl, heteroalkyl, haloheteroalkyl, aryl, heteroaryl, heterocycle, a protecting group, a mono-, di- or polyhydroxyalkyl residue, a mono-, di-, or polyhydroxyaryl residue, or a functional group of less than about 100,000 daltons, and n is an integer ranging from 0 to 4; and
   aryl or substituted aryl, which may optionally bear one or more substituents with a molecular weight of less than or equal to about 100,000 daltons;
   R₂ and R₃ can be the same or different and are selected from H, alkyl, cycloalkyl, aryl, (CH₂)_{w}OH, (CH₂)ₙO-alkyl, (CH₂)ₙOCOCH₃, CH₂CH(OH)CH₂OH, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, ((CH₂)ₙO)ₘ(CH₂)_{Q}OH, ((CH₂)ₙO)ₘ(CH₂O)_{Q}COCH₃, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN((CH₂)ₘN(CH₃)₂)₂, (CH₂)ₙN((CH₂)ₘN(CH₃)₃⁺A)₂, ((CH₂)ₙO)ₘ(CH₂)_{Q}OH, ((CH₂)ₙO)ₘ(CH₂O)_{Q}COCH₃, (CH₂)ₙO-haloalkyl, (CH₂)ₙN((CH₂)ₘO-alkyl)₂, (CH₂)ₙN((CH₂)ₘO-alkylether)₂, a mono-, di- or polyhydroxyalkyl residue, CH₂CH(OAc)CH₂OAc, an alkylphosphate residue, an alkylsulfonic acid residue, an alkylsulfonic ester or amide reside, an alkylmorpholine residue, an alkylheterocyclic residue, an alkylthiol residue, a mono-, di-, or polyhydroxyaryl residue, a mono-, di-, or polyetheralkyl residue, a mono-, di-, or polyetheraryl residue, and Q, n and may be the same or different and are integers ranging from 0 to 10,000, w is 1 or an integer ranging from 3 to 10,000, and A is a charge balancing ion;

In formula II, M can be selected from 2H, a metal cation, or photoactive metal ions preferably selected from Ga³⁺, Pt²⁺, Pd²⁺, Sn⁴⁺, In³⁺, Ge⁴⁺, Si⁴⁺, Al³⁺, Zn²⁺, and Mg²⁺, wherein optionally associated with the metal ion is the appropriate number of physiologically acceptable charge balancing counter ions.

In accordance with the invention, another preferred class of chlorins that may be used in a medicament for treatment of ophthalmic diseases or other disease indications is shown below:

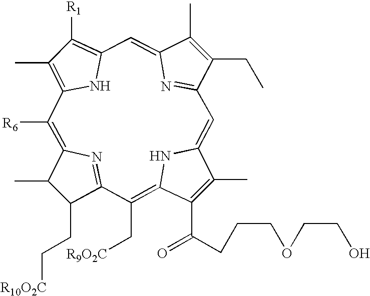

In this preferred class of chlorins:
R₁ is selected from H, CH₃, CH₂CH₃, CH═CH₂, CH₂OH, CH₂OAc, CH₂O-alkyl, CH₂O-alkoxy, CH═CHCH₂N(CH₃)₂, CH═CHCH₂N(CH₃)₃⁺A⁻, COCH₃, CHO, CH(OH)CH₃, CH(O-alkyl)CH₃, CH(O-alkoxy)CH₃, CH₂CH₂O-alkyl, CH₂CH₂O-alkoxy, and CH₂CH₂OAc, where A is a charge balancing ion;
R₆ is selected from H, halogen, and methyl;
R₉ is selected from alkyl, aryl, hydroxyalkyl, H, and a physiologically acceptable counter ion; and
R₁₀ is selected from alkyl, aryl, H, and a physiologically acceptable counter ion.

### DETAILED DESCRIPTION OF THE PREFERRED EMBODIMENTS

The ring opening of pheophorbides with ammonia, primary and secondary amines (Scheme 1) to form 6-carboxamides was first reported by Hans Fischer and co-workers in 1936 (H. Fischer, S. Goebel, Ann. Chem., 524, 269, 1936). Throughout the disclosure Fischer's nomenclature will be used for the numbering of group positions. In the experimental section, IUC nomenclature will be used. 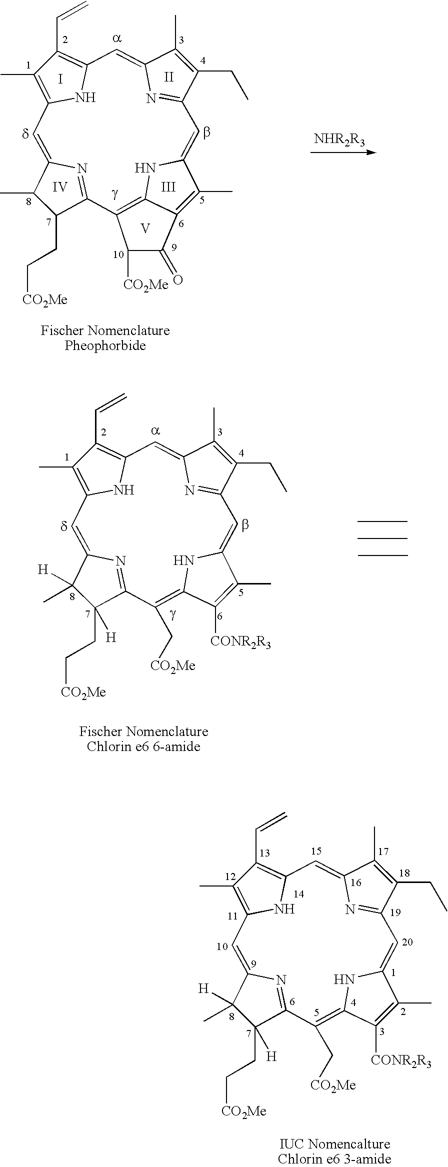
Scheme 1. Positional Numbering Systems for Pheophorbides and Chlorin e6 Analogs.

While being of some academic interest, the generality of the reaction in its application to designing photoreactive molecules for photodynamic therapy and in particular ophthalmic disease indications has resulted in it being largely ignored. As part of our ongoing investigations into designing photoactivated medicines for the field of photodynamic therapy, we have discovered that many chlorin e6 type 6-amide derivatives are surprisingly effective at closing and sustaining closure of corneal neovessels and neovessels of the choriocapillaris in advanced animal models. Indeed, many of these compounds display rapid elimination from skin, making them potentially valuable phototherapeutic compounds to treat diseases of the skin or eyes.

### Synthesis

The compounds of the instant invention rely on the selective opening of the five membered pheophorbide ring with primary or secondary amine containing compounds as shown in Scheme 1. As such, pheophorbides and functionalized derivatives thereof constitute effective starting materials for the production of a large number of functionalized chlorin e6 6-amide derivatives. The synthesis of several functionalized pheophorbides that act as photosensitizing agents themselves or as starting materials for the production of the modified chlorin e6 amides are outlined in Scheme 2.

### Methyl Pheophorbide (1)

Methyl pheophorbide a (Scheme 2, compound (1), X═H) is an abundant starting material for the synthesis of derivatized pheophorbides as well as the synthesis of chlorin e6 amide analogs. Methyl pheophorbide b, like methyl pheophorbide a except it contains a formyl group in the 3-position, may also be used according to the invention. The derivatized pheophorbides may then be ring opened according to the invention to produce functionalized chlorin e6 6-amides in which the amide unit is regioselectively introduced only at the 6-position. FIG. 1 shows the positions for chemical reactivity of methyl pheophorbide a or b according to classical pheophorbide chemistry.

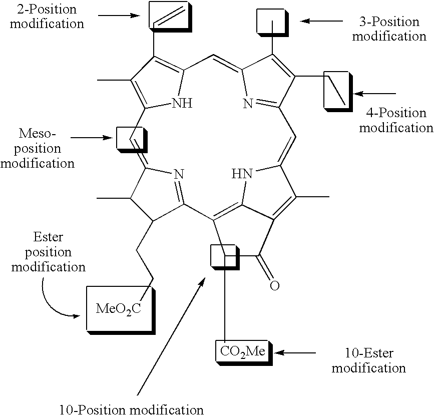

FIG. 1. Chemical Modification of Pheophorbide (1) (Fischer nomenclature used).

### Synthesis of Functionalized Pheophorbides

### 1.0 2-Position Modification

Scheme 2 outlines the modification of methyl pheophorbide a (compound (1), (X═H)) to produce pheophorbide derivatives, for example compounds (2)-(13), functionalized at the 2-position. Such modifications are described in the literature and are briefly summarized below.

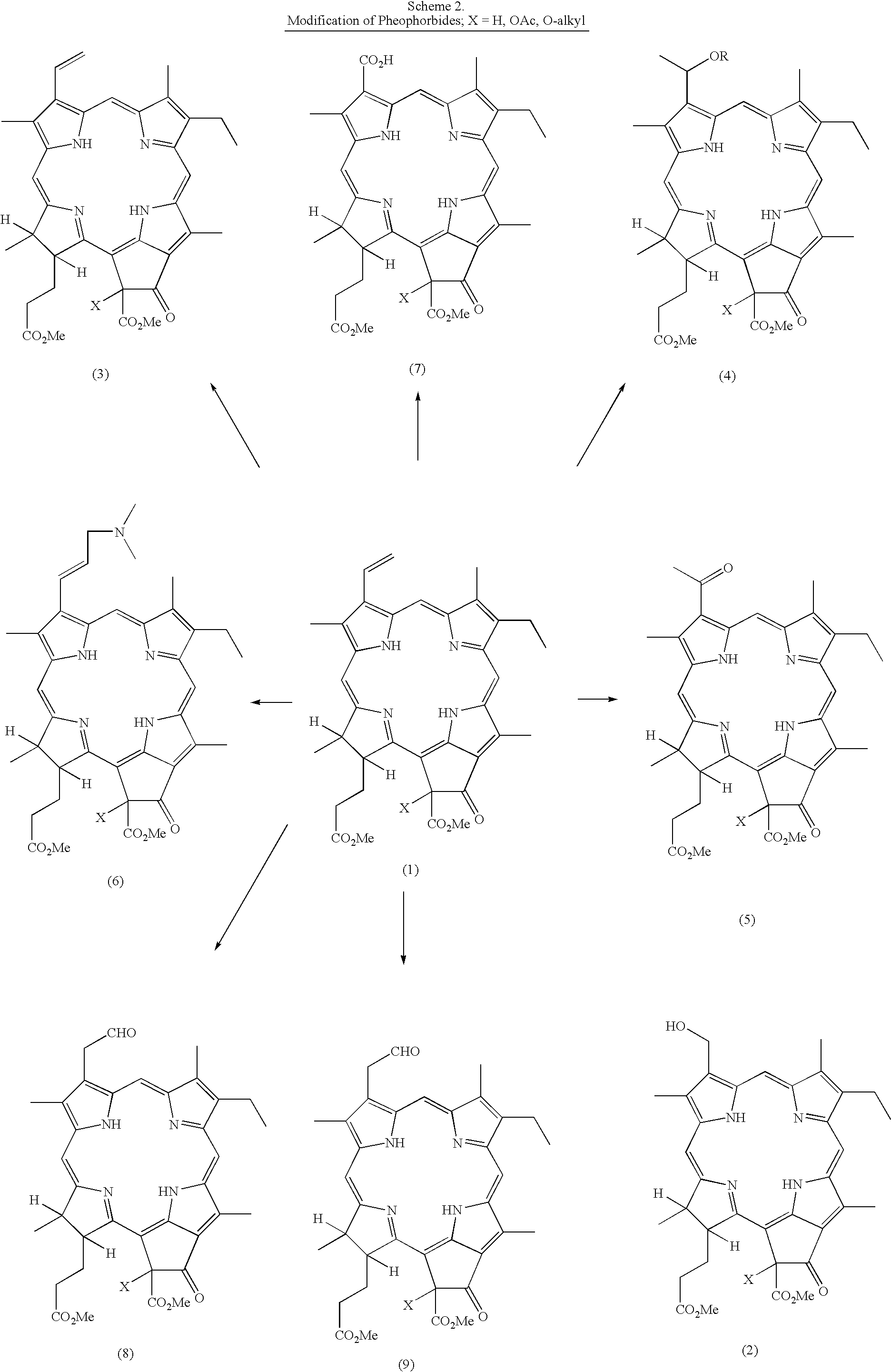

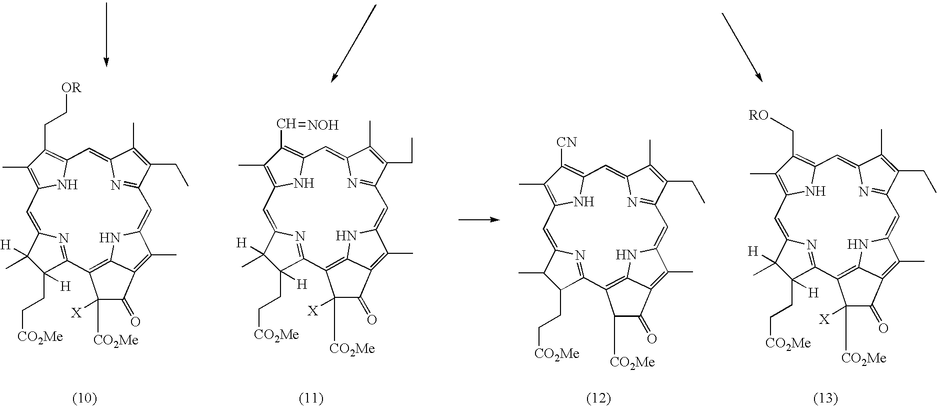

### 1.1 Vinyl Group Hydrogenation

Methyl pheophorbide (1) may be hydrogenated using H₂ Pd/C to give the corresponding meso-methylpheophorbide (3).

### 1.2 Alkylalcohols, Alkylethers and Alkylformyl Groups

Alternatively, (1) may be treated with 33% HBr/AcOH , which converts the vinyl group to a reactive 1′-bromo ethyl group. The bromine in this intermediate may be replaced via the addition of either alcohols or water to give the pheophorbide derivative (4) where R═H (when hydrolyzed by water; X═H), or functionalized ether derivatives (4) where R═R (X═H), depending on the alcohol used. Reaction of methyl pheophorbide with Tl(NO₂)₃ in methanol, followed by acid hydrolysis, yields the 2-desvinyl-2-(formylmethyl) pheophorbide (8) (X═H), which on reduction with sodium borohydride for example, yields the 2-desvinyl-2-(2-hydroxyethyl)pheophorbide (10) (R═H, X═H). 2-Desvinyl-2-formyl pheophorbides such as (9) (synthesis described in Section 1.5) may be reduced to yield 2-desvinyl-2-(hydroxymethyl)pheophorbides (2), X═H. The hydroxy group of (2) may then be converted to other functionalities (ester, ether, tosylate, etc.) via standard well recognized organic techniques known to those skilled in the art to produce derivatized pheophorbides, such as (13) (X═H), where R is the introduced functionality.

### 1.3 Acetyl Groups

The 2-desvinyl-2-(hydroxymethyl) pheophorbide (4) (R═H, X═H) may be converted to the 2-desvinyl-2-acetyl pheophorbide derivative (5) via selective oxidation with, for example, acetic anhydride/dimethylsulfoxide.

### 1.4 Formyl, Oxime and Cyano Groups

The synthesis of the 2-desvinyl-2-formyl pheophorbide (9) (X═H) from (1) follows several synthetic methodologies, either KMnO₄ oxidation, OsO₄/morpholine N-oxide/NalO₄, or more simply by ozonolysis. Alternatively, as previously mentioned, reaction of methyl pheophorbide with Tl(NO₂)₃ in methanol, followed by acid hydrolysis yields the 2-desvinyl-2-(formylmethyl) pheophorbide (8) (X═H), which may again be modified if desired.

Reaction of formyl pheophorbides with hydroxylamine at room temperature rapidly converts formyl groups to the oximes. If the reaction is monitored closely, or done at higher dilution conditions, very little if any ring V opening occurs. The oxime pheophorbides may then be dehydrated to give the corresponding cyanopheophorbides such as, for example, compound (12) (X═H), Scheme 2.

### 1.5 N,N-dimethylaminovinyl Groups

Methyl pheophorbide (1) (X═H) may be reacted with eschenmoser's salt to produce 2-desvinyl-2-(2-(N,N-dimethylaminomethyl)vinyl) pheophorbides like (6) (X═H). Once formed, these compounds may be reacted with iodonated reagents like iodomethane to produce, for example, 2-desvinyl-2-(2-(N,N,N-trimethylammoniummethyl)vinyl) pheophorbide analogs.

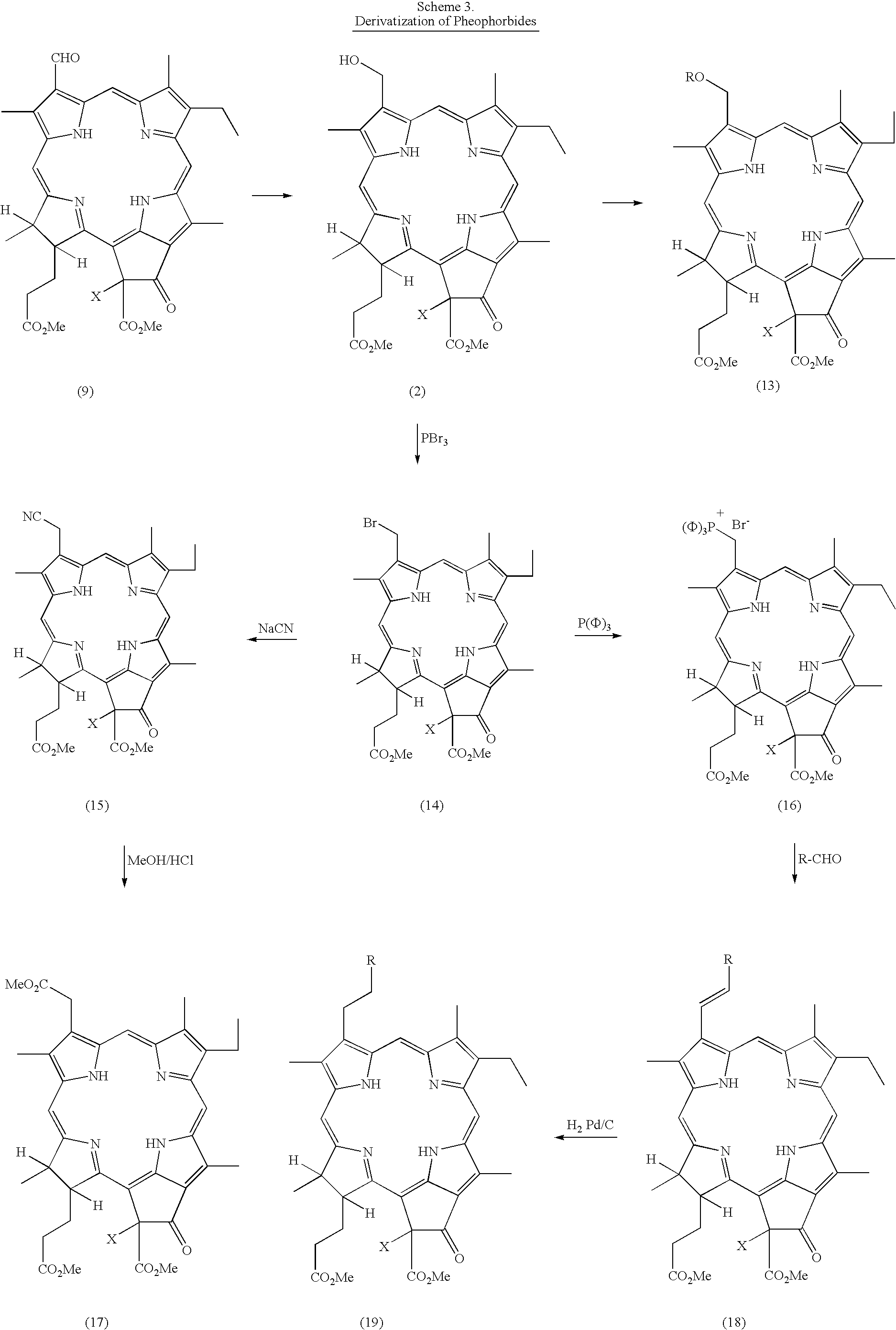

### 1.6 Haloalkyl, Cyanoalkyl Groups

2-Desvinyl-2-(2-hydroxyethyl)pheophorbides (compound 10, R═H) or 2-desvinyl-2-(hydroxymethyl)pheophorbide (2) derivatives react with thionyl bromide, carbon tetrabromide/triphenylphosphine or phosphorus tribromide to afford the 2-desvinyl-2-(2-bromoethyl)pheophorbide (for example compound (19) (R═Br, X═H, Scheme 3), or 2-desvinyl-2-(bromomethyl)pheophorbide derivatives (for example compound (14) (X═H), Scheme 3). Other halogenated pheophorbides can be made using other halogenating reagents. Treatment of such derivatives with sodium cyanide gives the corresponding 2-cyanoethyl compounds (for example compound (15) (X═H)), which can be further derivatized to produce acids, esters (for example compound (17)), amides or amines and the like. Alternatively, the bromine on these groups can be displaced with other nucleophiles to produce further derivatized pheophorbides.

### 1.7 Phosphonium Salts

The 2-desvinyl-2-(2-bromoethyl)pheophorbides (for example compound (19) (R═Br, X═H) or 2-desvinyl-2-(bromomethyl)pheophorbides (compound (14), X═H) may be reacted with triphenylphosphorane to produce the corresponding phosphonium salts (for example (16) (X═H). These may in turn be reacted with compounds containing a formyl group either aromatic or not, to produce the corresponding 2-desvinyl-2-(2-substituted vinyl) pheophorbides (for example compound (18), R=substituent, X═H, Scheme 3). Such derivatives may then be hydrogenated, if desired, to produce 2-desvinyl-2-(2-substituted alkyl) pheophorbide analogs (for example compound (19), X═H).

### 1.8 Esters and Amides

The 2-desvinyl-2-formylpheophorbides (9) under appropriate oxidizing conditions (KMnO₄/acetone) are converted to the 2-acid pheophorbide analogs, for example compound (7), X═H, Scheme 2. The 2-carboxylic acid derivatives may then be converted to an ester of an amide via standard chemistry.

### 2.0 δ-Meso-Position Modification

### 2.1 Halogenation

The meso-position directly adjacent to the reduced pyrrole ring can be functionalized with a variety of reagents. Meso-halogenation with Br or Cl, for example, occurs at the δ-meso-position using halogenated-succinamides, or with HCl to produce meso-halogenated pheophorbides. F and I may also be introduced into the meso position.

### 2.2 Formylation and Methylation

Chlorin e6 trimethyl ester (20) (Scheme 4) may be metallated with either copper or nickel to produce compound (21), which can be reacted with Vilsmeier reagent to give the metallo-meso-formyl chlorin e6 derivative (22). The formyl group may then be converted to a methyl group via reaction with NaBH₄ in TFA (compound 23). Demetallation of the meso-methyl metallochlorin e6 derivative (24) and subsequent treatment with sodium methoxide in methanol/acetone gives δ-methyl pheophorbide (25). The pheophorbide (25) may then be reacted with amines according to the invention or further modified according to the chemistries outlined in Scheme 2, prior to reaction with amines. Similar peripheral functional group modifications as shown in Scheme 2 are possible on chlorin e6 trimethyl ester itself, and following basic treatment functionalized pheophorbides may be generated. Pheophorbides possessing δ-formyl groups are known in the literature.

### 3.0 Propionic Ester Position Modification

The propionic acid side chain on pheophorbides may be modified to produce esters using standard esterification techniques well known to those skilled in the art. The formation of amides is possible using coupling reagents like ethylchloroformate, 1,3-dicyclohexylcarbodiimide or carbonyl diimidazole and the like without opening the cyclic ring system. In this way a vast variety of propionic amide pheophorbides may be generated and subsequently ring opened to form functionalized chlorin e6 analogues according to the invention. Particularly preferred amides are alkylamides and amides containing heteroatoms. 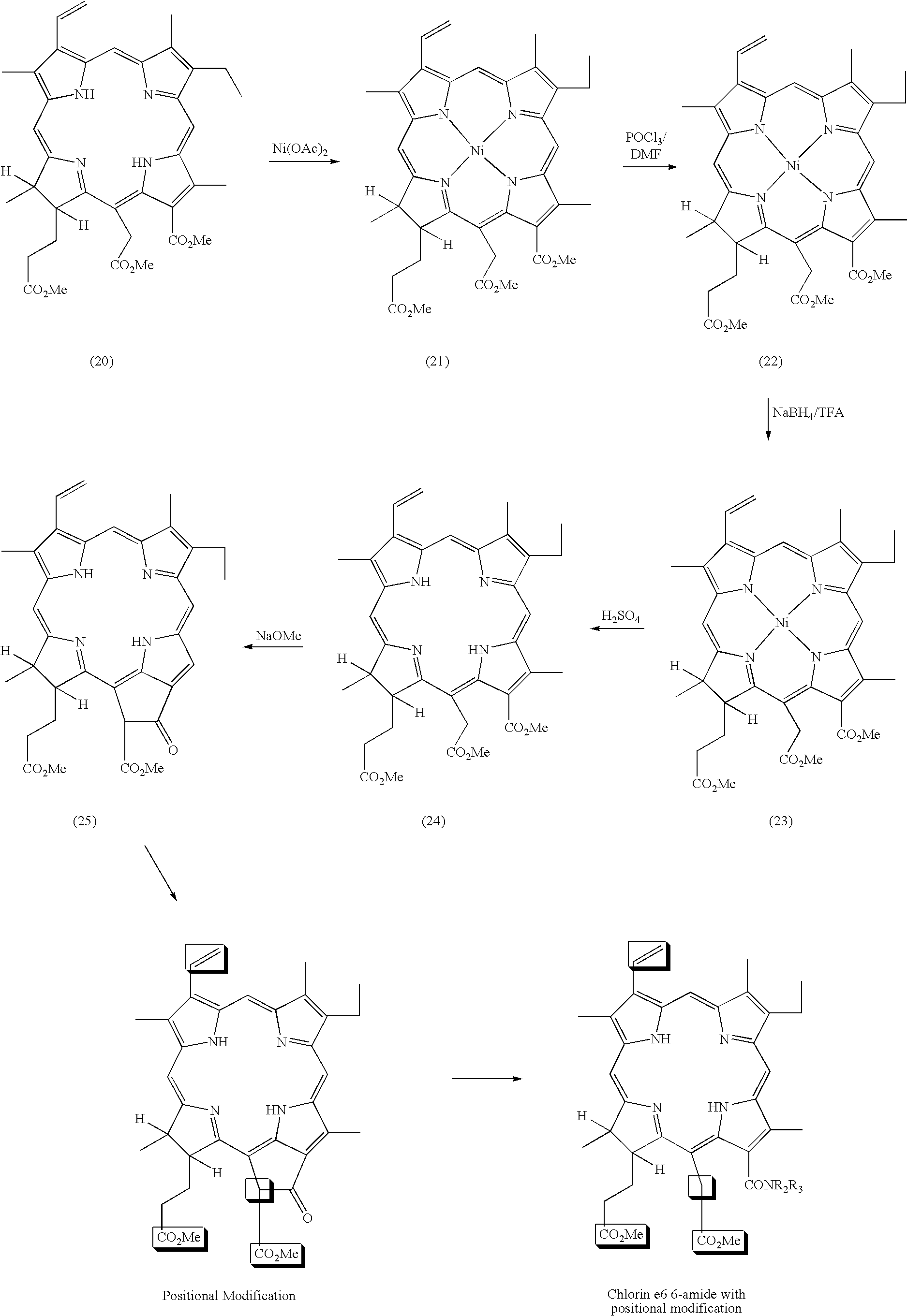
Scheme 4. Chlorin e6 Modification with Conversion to Pheophorbides.

In addition to esters and amides, the propionic ester group may be hydrolyzed under acidic conditions to form carboxylic acids and converted, if desired, to carboxylic acid salts.

### 4.0 10-Position Modification

Pheophorbides undergo chemical modification at the 10-position on ring V. Substituents such as X═OH, OMe, OEt, O-alkyls and acetate have been successfully introduced onto the 10-position.

### 5.0 10-Ester Modification

U.S. Pat. No. 4,709,022, the disclosure of which is hereby incorporated by reference herein, outlines the synthesis of 10-ester modified pheophorbide derivatives. In this instance, the methyl group on the ester is replaced by ethylene glycol. Such ester modifications should be possible with a wide variety of esters, thus producing 10-ester modified pheophorbides. Outlined also in U.S. Pat. No. 4,709,022 is the formation of 10-amide pheophorbides. Such amide modifications should be possible with other amino acid types, thus producing 10-ester modified pheophorbides. Both the 10-ester modified and 10-amide analogs can be ring opened according to the invention.

### 6.0 4-Position Modification

Pheophorbides may be generated that possess in addition to ethyl, groups such as formyl, vinyl, CH(OH)CH₃, CH₂OH, CH(O-alkyl)CH₃, in the 4-position. It is envisaged that similar modifications to such groups as outlined in Scheme 2 above are possible for the 4-position.

### 7.0 3-Position Modification

Methyl pheophorbide b, which possesses a formyl group in the 3-position, serves as a convenient starting material for the synthesis of 3-modified pheophorbides. Through modification of the formyl moiety, groups such as vinyl, CH(OH)CH₃, CH₂OH, CH(O-alkyl)CH₃, CH₂O-alkyl can be introduced to the 3-position. It is envisaged that similar modifications to groups on the 3-position are possible, as outlined above for modifications to the 2-position, thus producing a large number of functionalized pheophorbides.

### Pheophorbides with Multiple Position Modifications

Clearly, it is possible to produce pheophorbides that have one or more of the positions outlined in FIG. 1 that are modified; that is, combinations of modifications from that observed with methyl pheophorbide. Such pheophorbides are within the scope of the invention.

### Synthesis of Diester Chlorin e6 Amides from Diester Pheophorbides

Methyl pheophorbides a & b, compounds outlined in Schemes 2-4 and combinations thereof, as well as pheophorbides with modifications to the periphery as outlined above, serve as starting materials for further derivatization according to the invention. Reaction of pheophorbides with compounds containing an amine functionality form chlorin e6 mono-amide diester derivatives (26) (Scheme 5). Such derivatives may then, if desired, be metallated to produce metallochlorin e6 mono-amide diester derivatives using metallation techniques outlined in “The Porphyrins” Ed. D. Dolphin, Vol I, Chapter 10, p 389-483, Academic Press, the disclosure of which is hereby incorporated by reference herein. Similar chemistry may be undertaken using different ester derivatives of methyl pheophorbide (1), for example, ethyl pheophorbide. In this instance, chlorin e6 diester monoamine derivatives may be synthesized that possess different ester functionalities. This may be important for adjusting the lippophilic properties of the molecules. These compounds may additionally be metallated to produce metallo-complexes. 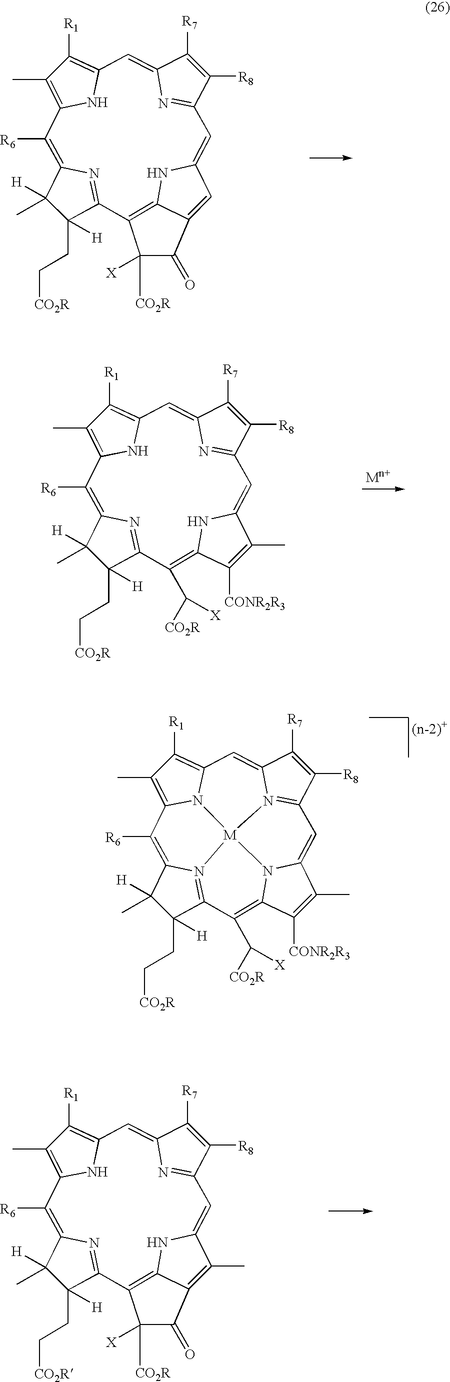 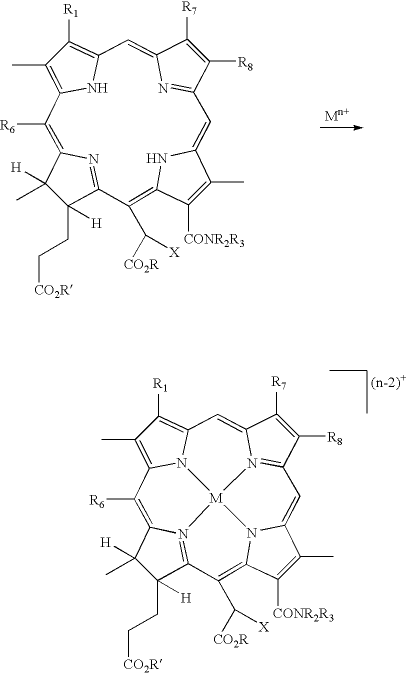
Scheme 5. Chlorin e6 Monoamide Diester Synthetic Routes.

### Synthesis of Mono-ester Mono-acid Chlorin e6 Amides from Mono-ester Pheophorbides

Clearly, in addition to the peripheral modifications to pheophorbides outlined above, the propionic ester on the pheophorbides may be hydrolyzed with HCl/water to give mono-carboxylic acid pheophorbides (for example (27), Scheme 6), which may then be ring opened with an amine of choice to form chlorin e6 monoacid, mono-ester, or mono-amide derivatives such as (28). These may then be metallated, if desired, to give the corresponding metallo-derivatives. Under controlled basic conditions that do not cleave the ester, the chlorin e6 monoacid, mono-ester, mono-amide derivatives may be converted to the corresponding sodium or potassium salts. Alternatively, the acetic acid ester on (28) may be further hydrolyzed to produce di-acid (or di-salt) chlorin e6 mono-amides. 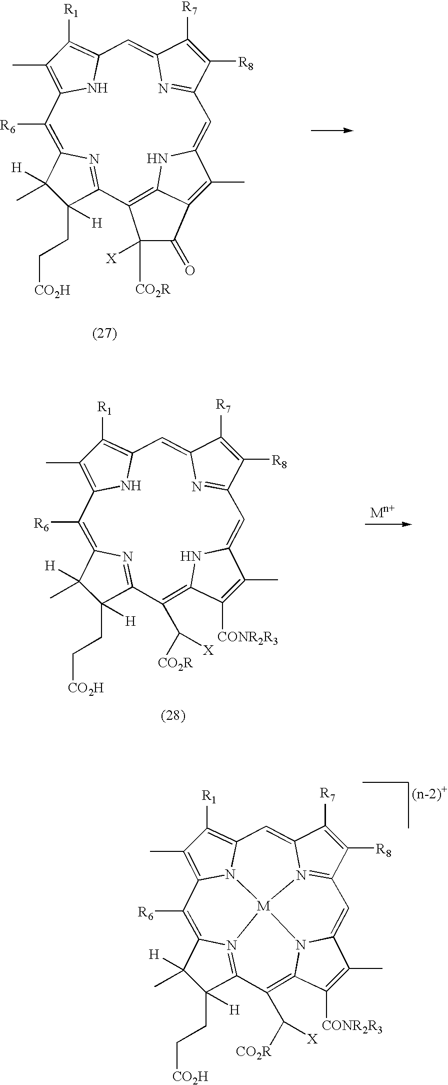
Scheme 6: Synthesis of Chlorin e6 Monoacid, Mono-ester, Mono-amide Derivatives

### Synthesis of Mono-ester Chlorin e6 Di-amides from Mono-ester Pheophorbides or Mono-ester Mono Acid Chlorin e6 Amides

The propionic acid group on mono-acid pheophorbides (27) (such as pheophorbide a) may be converted to an amide moiety by careful use of coupling reagents and the desired amine (Scheme 7). A preferred coupling reagent is ethyl chloroformate, which in the presence of the mono-acid pheophorbide and triethylamine forms the corresponding mixed anhydride. This reacts quickly with amines to form the desired amide pheophorbides. In general, the competitive ring opening reaction with pheophorbide and the amine is a much slower process than is the formation of the propionic amide, especially if the reaction is undertaken in dilute solution. In this way it is possible to generate chlorin e6 di-amide derivatives that possess two different amide groups or identical functionalities. 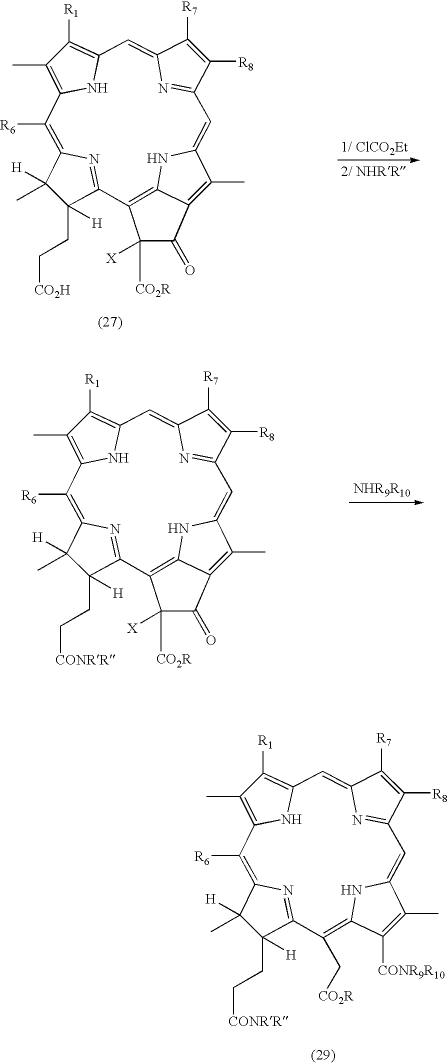
Scheme 7: Synthesis of Chlorin e6 Monoester, Di-amide Derivatives from Pheophorbides

Alternatively, the mono-acid chlorin e6 amide derivatives like (28) may be converted to chlorin e6 diamide derivatives (29) by reaction of the propionic acid group with amines and coupling reagents like ethyl chloroformate as described above. In this instance, chlorin e6 derivatives bearing the same amine functionalities can be produced or compounds bearing two different amine functionalities.

### Synthesis of Mono-acid Chlorin e6 Di-amides from Mono-ester Chlorin e6 Di-amides

The acetic ester moiety on the mono-ester chlorin e6 di-amides derivatives (for example (29) Scheme 7) may be further hydrolyzed to produce mono-acid diamide chlorin e6 derivatives (29) (R═H). Chlorin e6 derivatives (28) may then be converted to esters or to amides or salts via the methods described above.

### Synthesis of Chlorin e6 Tri-amides from Diester Chlorin e6 Amides, Mono-ester Monoacid Chlorin e6 Amides and Diacid Chlorin e6 Amides

The di-ester mono-amide chlorin e6 derivatives of general structure (26) may be further modified by hydrolyzing the ester functionalities to form the diacid (or salts thereof) chlorin e6 compounds. These may, in turn, be converted to triamide chlorin e6 derivatives via coupling reactions or via conversion of the acids to the acid chlorides and subsequent reaction with amines. In this instance, chlorin e6 triamides may be produced having identical amide groups, or having two of the amide groups being the same, while the third is different.

Alternatively, the mono-ester monoacid chlorin e6 amides may be converted to mono-ester chlorin e6 diamides (for example (29)) via the chemistry described above, which may, if desired, possess different amide functionality. The remaining ester functionality may then be hydrolyzed and reacted with a third amine to produce the chlorin e6 tri-amide derivatives.

### Reduction of the Esters on the Chlorin e6 Amides to Produce Alkylalcohol Chlorin e6 Amide Derivatives

As a further extension of the chemistry, Scheme 8 outlines the synthesis of mono- and di-alcohol 6-amide chlorin derivatives. LiBH₄ effectively reduces the esters to alcohols without reduction of the amide group. It is known that carbonyl imidazole moieties may be reduced with NaBH₄ thus producing mono-alcohol, mono-ester, or mono amide derivatives. In this way, mono and di-alcohol chlorin e6 analogs may be synthesized, which may be metallated, if desired, or further modified by chemical modification of the alcohol moieties. The alcohol moieties may, if desired, be reacted chemically by techniques well known to those skilled in the art to produce esters, ethers, phosphates, phosphonates, sulfonyl esters and the like, protected with protecting groups, or reacted with well recognized leaving groups. Such groups may then be further modified as outlined in Scheme 9, the chemistry of which is well known to those skilled in the art.

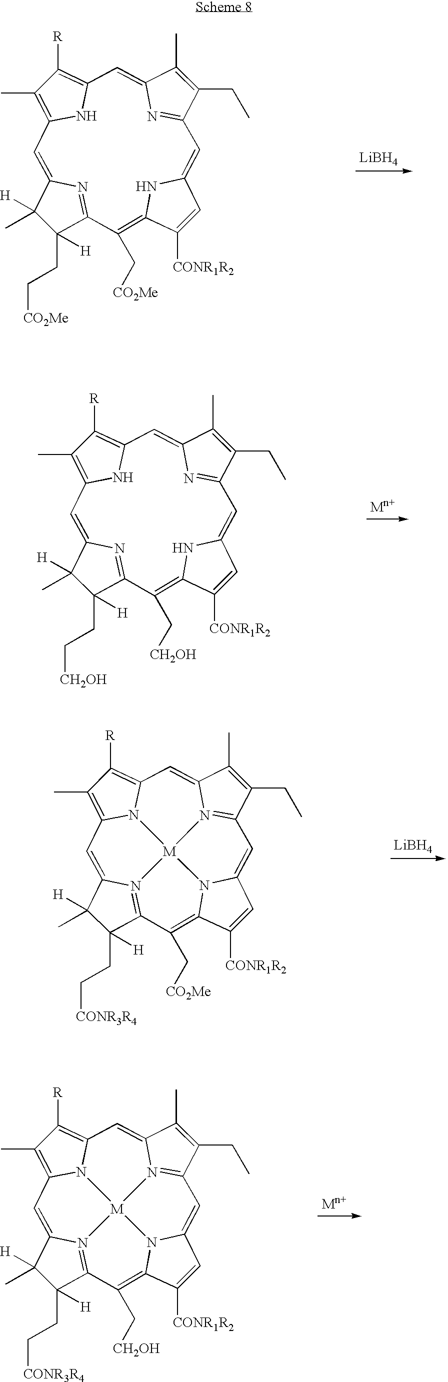

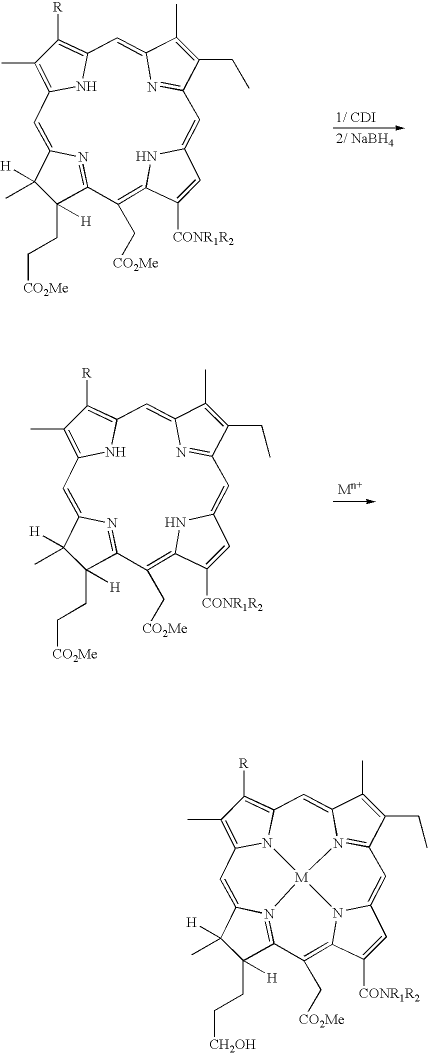

It should be recognized that such functional modifications to hydroxyl groups as outlined in Scheme 9 are also possible on positions R₁, R₂, R₃, R₄, R₆, R₇, R₈ and other peripheral positions on the chlorin ring system in FIG. 1 and II. 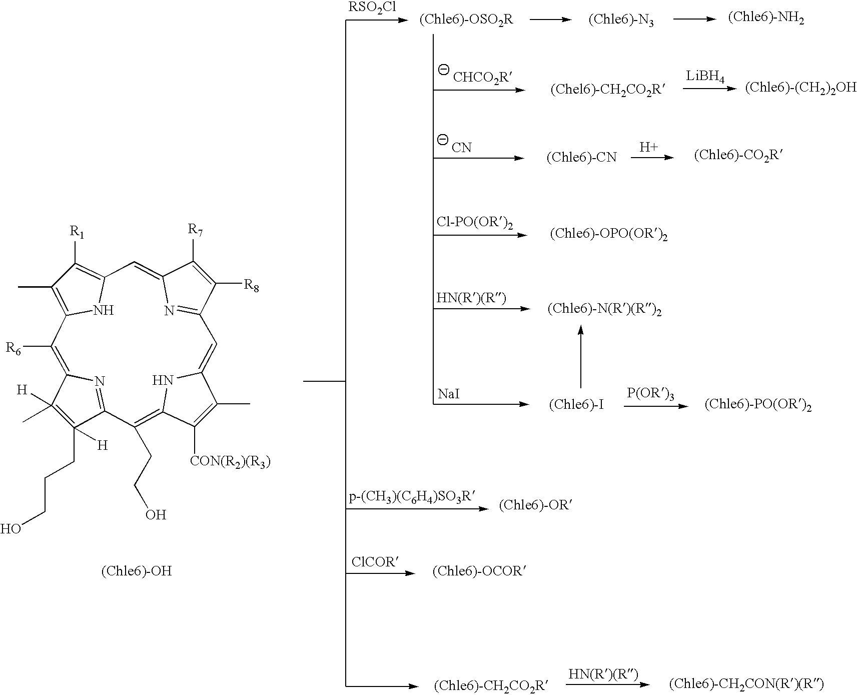
Scheme 9. Functionalization of Hydroxychlorin e6 Derivatives.

### Modification of Chlorin e6 Amide Periphery Groups

Similarly, as explained with pheophorbides, the periphery of the formed chlorin e6 derivatives may be further modified chemically to introduce desired functional groups (Scheme 10). Once a particular amide group has been incorporated onto the 13-position according to the invention, a large number of chemical modifications to the chlorin e6 ring system are possible, similar to that outlined above for the pheophorbide modification. Such derivatization allows for modifications to lipophilicity or allows attachment of biomolecules of interest (for example antibodies and the like), and is within the scope of the present invention. Derivatization of the chlorin e6 amide after formation from the corresponding pheophorbide allows for chemical manipulation that may not be possible on the pheophorbides themselves (for example meso-formylation). As disclosed herein, pheophorbides efficiently undergo reactions with primary and secondary amines to produce chlorin e6 amides via ring opening. Table 1 outlines the synthesis of representative chlorin e6 amides with some primary amines according to the invention. Table 1 is not intended to limit the invention in any way, but merely illustrates some embodiments of the invention.

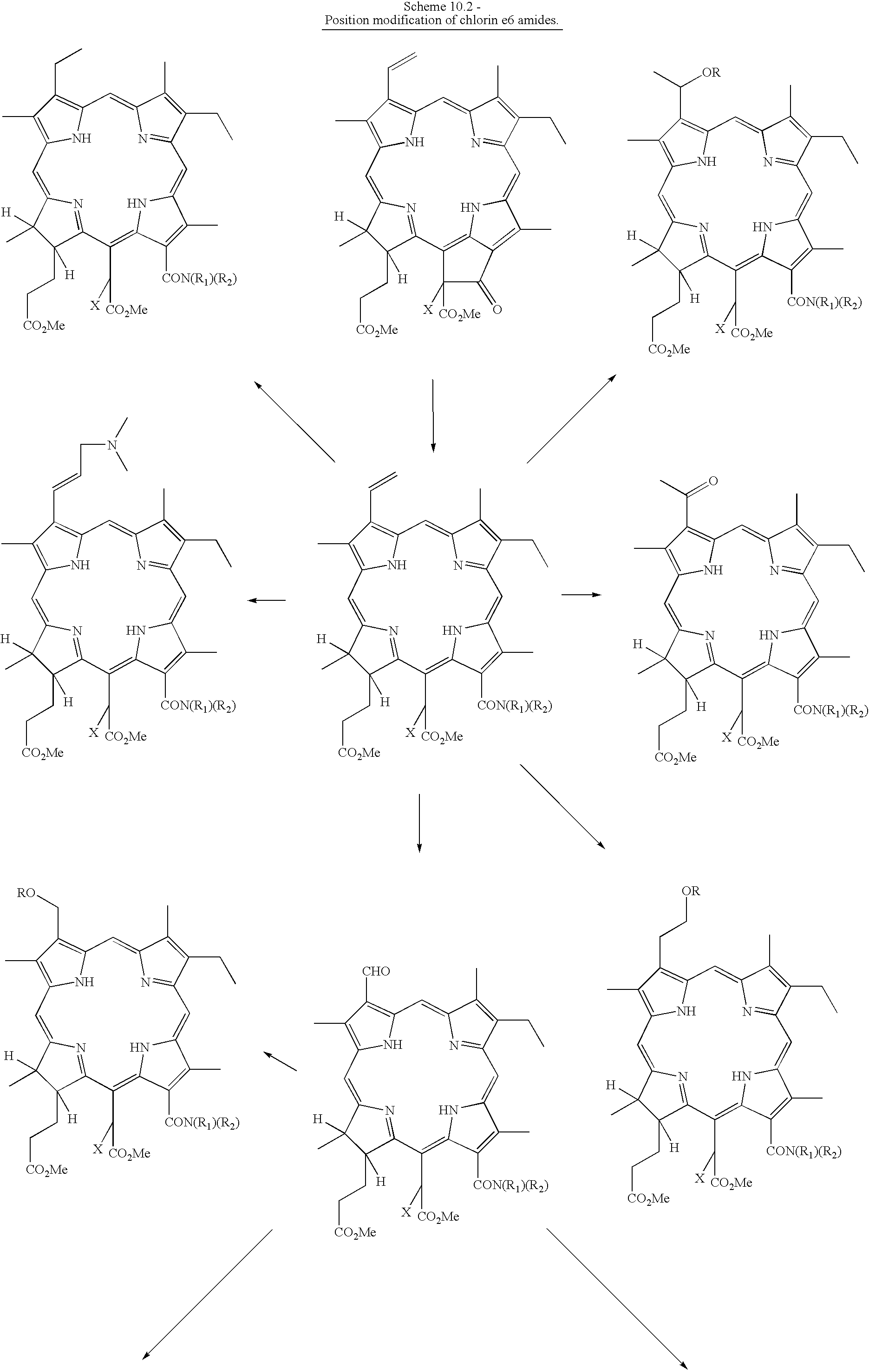

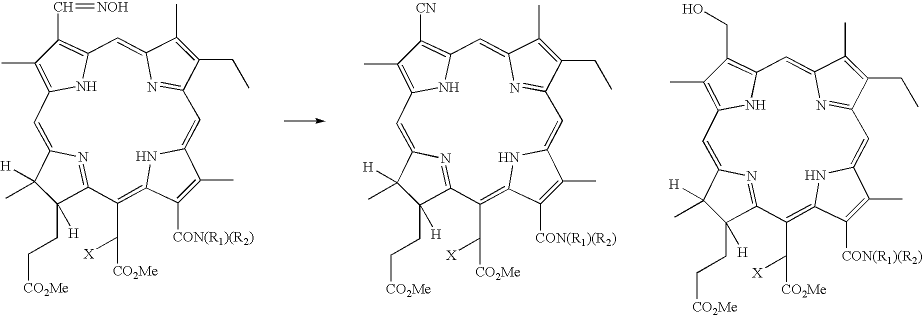

TABLE 1
Vinyl chlorine e6 derivatives; M = H2

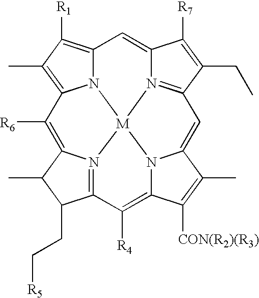

| Compound No | R₁ | R₂ | R₃ | R₄ | R₅ | R₆ | R₇ |
|---|---|---|---|---|---|---|---|
| 30 | V | H | (CH₂)₃OH | CH₂CO₂Me | CO₂Me | H | CH₃ |
| 31 | V | H | (CH₂)₃OH | CH₂CO₂H | CO₂H | H | CH₃ |
| 32 | V | H | (CH₂)₃OH | CH₂CO₂Na | CO₂Na | H | CH₃ |
| 33 | V | H | (CH₂)₃OH | CH(OH)CO₂Me | CO₂Me | H | CH₃ |
| 34 | V | H | (CH₂)₃OAc | CH₂CO₂Me | CO₂Me | H | CH₃ |
| 35 | V | H | (CH₂)₃OH | CH₂CO₂Me | CONHMe | H | CH₃ |
| 36 | V | H | (CH₂)₃OH | CH₂CH₂OH | CONHMe | H | CH₃ |
| 37 | V | H | (CH₂)₃OH | CH₂CH₂OH | CH₂OH | H | CH₃ |
| 38 | V | H | CH₂CH(OH)CH₂OH | CH₂CO₂Me | CO₂Me | H | CH₃ |
| 39 | V | H | (CH₂)₂O(CH₂)₂OH | CH₂CO₂Me | CO₂Me | H | CH₃ |
| 40 | V | H | (CH₂)₂O(CH₂)₂OH | CH₂CO₂H | CO₂H | H | CH₃ |
| 41 | V | H | (CH₂)₂O(CH₂)₂OH | CH₂CO₂Na | CO₂Na | H | CH₃ |
| 42 | V | H | (CH₂)₂O(CH₂)₂OH | CH(OH)CO₂Me | CO₂Me | H | CH₃ |
| 43 | V | H | (CH₂)₂O(CH₂)₂OH | CH₂CH₂OH | CH₂OH | H | CH₃ |
| 44 | V | H | (CH₂)₂O(CH₂)₂Ome | CH₂CO₂Me | CO₂Me | H | CH₃ |
| 45 | V | H | (CH₂)₂O(CH₂)₂Oac | CH₂CO₂Me | CO₂Me | H | CH₃ |
| 46 | V | H | (CH₂)₂O(CH₂)₂NH₂ | CH₂CO₂Me | CO₂Me | H | CH₃ |
| 47 | V | H | (CH₂)₂O(CH₂)₂N(CH₃)₂ | CH₂CO₂Me | CO₂Me | H | CH₃ |
| 48 | V | H | (CH₂)₂O(CH₂)₂N(CH₃)₃⁺I⁻ | CH₂CO₂Me | CO₂Me | H | CH₃ |
| 49 | V | H | (CH₂)₃N(CH₂CH₂OH)₂ | CH₂CO₂Me | CO₂Me | H | CH₃ |
| 50 | V | H | (CH₂)₃N(CH₂CH₂OMe)₂ | CH₂CO₂Me | CO₂Me | H | CH₃ |
| 51 | V | H | (CH₂)₃N(CH₂CH₂OAc)₂ | CH₂CO₂Me | CO₂Me | H | CH₃ |
| 52 | V | H | (CH₂)₂O(CH₂)₂O(CH₂)₂OH | CH₂CO₂Me | CO₂Me | H | CH₃ |
| 53 | V | H | (CH₂)₂O(CH₂)₂O(CH₂)₂OMe | CH₂CO₂Me | CO₂Me | H | CH₃ |
| 54 | V | H | (CH₂)₃OPO₃H₂ | CH₂CO₂Me | CO₂Me | H | CH₃ |
| 55 | V | H | (CH₂)₃OSO₃H | CH₂CO₂Me | CO₂Me | H | CH₃ |

TABLE 2
Formyl chlorin e6 derivatives; M = H2

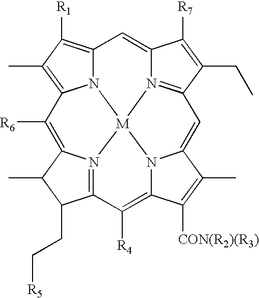

| Comp. No | R₁ | R₂ | R₃ | R₄ | R₅ | R₆ | R₇ |
|---|---|---|---|---|---|---|---|
| 56 | CHO | H | CH₃ | CH₂CO₂Me | CO₂Me | H | CH₃ |
| 57 | CHO | H | (CH₂)₄CH₃ | CH₂CO₂Me | CO₂Me | H | CH₃ |
| 58 | CHO | H | (CH₂)₂OH | CH₂CO₂Me | CO₂Me | H | CH₃ |
| 59 | CHO | H | (CH₂)₃OH | CH₂CO₂Me | CO₂Me | H | CH₃ |
| 60 | CHO | H | (CH₂)₃OH | CH(OH)CO₂Me | CO₂Me | H | CH₃ |
| 61 | CHO | H | (CH₂)₃OH | CH₂CO₂Me | CONHMe | H | CH₃ |
| 62 | CHO | H | CH₂CH(OH)CH₂OH | CH₂CO₂Me | CO₂Me | H | CH₃ |
| 63 | CHO | H | (CH₂)₂O(CH₂)₂OH | CH₂CO₂Me | CO₂Me | H | CH₃ |
| 64 | CHO | H | (CH₂)₂O(CH₂)₂OH | CH(OH)CO₂Me | CO₂Me | H | CH₃ |
| 65 | CHO | H | (CH₂)₂O(CH₂)₂Ome | CH₂CO₂Me | CO₂Me | H | CH₃ |
| 66 | CHO | H | (CH₂)₂O(CH₂)₂Oac | CH₂CO₂Me | CO₂Me | H | CH₃ |
| 67 | CHO | H | (CH₂)₂O(CH₂)₂NH₂ | CH₂CO₂Me | CO₂Me | H | CH₃ |
| 68 | CHO | H | (CH₂)₂O(CH₂)₂N(CH₃)₂ | CH₂CO₂Me | CO₂Me | H | CH₃ |
| 69 | CHO | H | (CH₂)₂O(CH₂)₂N(CH₃)₃⁺I⁻ | CH₂CO₂Me | CO₂Me | H | CH₃ |
| 70 | CHO | H | (CH₂)₃N(CH₂CH₂OH)₂ | CH₂CO₂Me | CO₂Me | H | CH₃ |
| 71 | CHO | H | (CH₂)₃N(CH₂CH₂OMe)₂ | CH₂CO₂Me | CO₂Me | H | CH₃ |
| 72 | CHO | H | (CH₂)₃N(CH₂CH₂OAc)₂ | CH₂CO₂Me | CO₂Me | H | CH₃ |
| 73 | CHO | H | (CH₂)₂O(CH₂)₂O(CH₂)₂OH | CH₂CO₂Me | CO₂Me | H | CH₃ |
| 74 | CHO | H | (CH₂)₂O(CH₂)₂O(CH₂)₂Ome | CH₂CO₂Me | CO₂Me | H | CH₃ |

TABLE 3
2-Hydroxymethyl chlorin e6 derivatives; M = H2

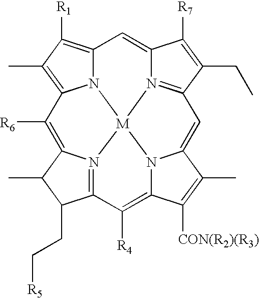

| Comp. No | R₁ | R₂ | R₃ | R₄ | R₅ | R₆ | R₇ |
|---|---|---|---|---|---|---|---|
| 75 | CH₂OH | H | CH₃ | CH₂CO₂Me | CO₂Me | H | CH₃ |
| 76 | CH₂OH | H | (CH₂)₅CH₃ | CH₂CO₂Me | CO₂Me | H | CH₃ |
| 77 | CH₂OH | H | (CH₂)₂OH | CH₂CO₂Me | CO₂Me | H | CH₃ |
| 78 | CH₂OH | H | (CH₂)₃OH | CH₂CO₂Me | CO₂Me | H | CH₃ |
| 79 | CH₂OH | H | (CH₂)₃OH | CH₂CO₂H | CO₂H | H | CH₃ |
| 80 | CH₂OH | H | (CH₂)₃OH | CH₂CO₂Na | CO₂Na | H | CH₃ |
| 81 | CH₂OH | H | (CH₂)₃OH | CH(OH)CO₂Me | CO₂Me | H | CH₃ |
| 82 | CH₂OH | H | (CH₂)₃OH | CH₂CO₂Me | CONHMe | H | CH₃ |
| 83 | CH₂OH | H | CH₂CH(OH)CH₂OH | CH₂CO₂Me | CO₂Me | H | CH₃ |
| 84 | CH₂OH | H | (CH₂)₂O(CH₂)₂OH | CH₂CO₂Me | CO₂Me | H | CH₃ |
| 85 | CH₂OH | H | (CH₂)₂O(CH₂)₂OH | CH₂CO₂H | CO₂H | H | CH₃ |
| 86 | CH₂OH | H | (CH₂)₂O(CH₂)₂OH | CH₂CO₂Na | CO₂Na | H | CH₃ |
| 87 | CH₂OH | H | (CH₂)₂O(CH₂)₂OH | CH(OH)CO₂Me | CO₂Me | H | CH₃ |
| 88 | CH₂OH | H | (CH₂)₂O(CH₂)₂Ome | CH₂CO₂Me | CO₂Me | H | CH₃ |
| 89 | CH₂OH | H | (CH₂)₂O(CH₂)₂Oac | CH₂CO₂Me | CO₂Me | H | CH₃ |
| 90 | CH₂OH | H | (CH₂)₂O(CH₂)₂NH₂ | CH₂CO₂Me | CO₂Me | H | CH₃ |
| 91 | CH₂OH | H | (CH₂)₂O(CH₂)₂N(CH₃)₂ | CH₂CO₂Me | CO₂Me | H | CH₃ |
| 92 | CH₂OH | H | (CH₂)₂O(CH₂)₂N(CH₃)₃⁺I⁻ | CH₂CO₂Me | CO₂Me | H | CH₃ |
| 93 | CH₂OH | H | (CH₂)₃N(CH₂CH₂OH)₂ | CH₂CO₂Me | CO₂Me | H | CH₃ |
| 94 | CH₂OH | H | (CH₂)₃N(CH₂CH₂OMe)₂ | CH₂CO₂Me | CO₂Me | H | CH₃ |
| 95 | CH₂OH | H | (CH₂)₃N(CH₂CH₂OAc)₂ | CH₂CO₂Me | CO₂Me | H | CH₃ |
| 96 | CH₂OH | H | (CH₂)₂O(CH₂)₂O(CH₂)₂OH | CH₂CO₂Me | CO₂Me | H | CH₃ |
| 97 | CH₂OH | H | (CH₂)₂O(CH₂)₂O(CH₂)₂Ome | CH₂CO₂Me | CO₂Me | H | CH₃ |

TABLE 4
Acetyl chlorin e6 derivatives; M = H2

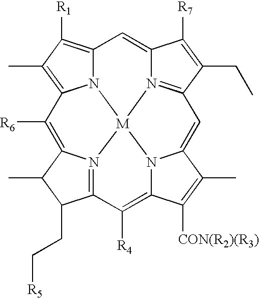

| Comp. No | R₁ | R₂ | R₃ | R₄ | R₅ | R₆ | R₇ |
|---|---|---|---|---|---|---|---|
| 98 | COCH₃ | H | CH₃ | CH₂CO₂Me | CO₂Me | H | CH₃ |
| 99 | COCH₃ | H | (CH₂)₂OH | CH₂CO₂Me | CO₂Me | H | CH₃ |
| 100 | COCH₃ | H | (CH₂)₅CH₃ | CH₂CO₂Me | CO₂Me | H | CH₃ |
| 101 | COCH₃ | H | (CH₂)₃OH | CH₂CO₂Me | CO₂Me | H | CH₃ |
| 102 | COCH₃ | H | (CH₂)₃OH | CH₂CO₂H | CO₂H | H | CH₃ |
| 103 | COCH₃ | H | (CH₂)₃OH | CH₂CO₂Na | CO₂Na | H | CH₃ |
| 104 | COCH₃ | H | (CH₂)₃OH | CH₂CO₂Me | CONHMe | H | CH₃ |
| 105 | COCH₃ | H | CH₂CH(OH)CH₂OH | CH₂CO₂Me | CO₂Me | H | CH₃ |
| 106 | COCH₃ | H | (CH₂)₂O(CH₂)₂OH | CH₂CO₂Me | CO₂Me | H | CH₃ |
| 107 | COCH₃ | H | (CH₂)₂O(CH₂)₂OH | CH₂CO₂H | CO₂H | H | CH₃ |
| 108 | COCH₃ | H | (CH₂)₂O(CH₂)₂OH | CH₂CO₂Na | CO₂Na | H | CH₃ |
| 109 | COCH₃ | H | (CH₂)₂O(CH₂)₂Ome | CH₂CO₂Me | CO₂Me | H | CH₃ |
| 110 | COCH₃ | H | (CH₂)₂O(CH₂)₂Oac | CH₂CO₂Me | CO₂Me | H | CH₃ |
| 111 | COCH₃ | H | (CH₂)₂O(CH₂)₂NH₂ | CH₂CO₂Me | CO₂Me | H | CH₃ |
| 112 | COCH₃ | H | (CH₂)₂O(CH₂)₂N(CH₃)₂ | CH₂CO₂Me | CO₂Me | H | CH₃ |
| 113 | COCH₃ | H | (CH₂)₂O(CH₂)₂N(CH₃)₃⁺I⁻ | CH₂CO₂Me | CO₂Me | H | CH₃ |
| 114 | COCH₃ | H | (CH₂)₃N(CH₂CH₂OH)₂ | CH₂CO₂Me | CO₂Me | H | CH₃ |
| 115 | COCH₃ | H | (CH₂)₃N(CH₂CH₂OMe)₂ | CH₂CO₂Me | CO₂Me | H | CH₃ |
| 116 | COCH₃ | H | (CH₂)₃N(CH₂CH₂OAc)₂ | CH₂CO₂Me | CO₂Me | H | CH₃ |
| 117 | COCH₃ | H | (CH₂)₂O(CH₂)₂O(CH₂)₂OH | CH₂CO₂Me | CO₂Me | H | CH₃ |
| 118 | COCH₃ | H | (CH₂)₂O(CH₂)₂O(CH₂)₂Ome | CH₂CO₂Me | CO₂Me | H | CH₃ |

TABLE 5
Ethyl chlorin e6 derivatives; M = H2

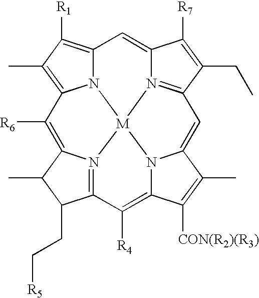

| Comp. No | R₁ | R₂ | R₃ | R₄ | R₅ | R₆ | R₇ |
|---|---|---|---|---|---|---|---|
| 119 | Et | H | CH₃ | CH₂CO₂Me | CO₂Me | H | CH₃ |
| 120 | Et | H | (CH₂)₅CH₃ | CH₂CO₂Me | CO₂Me | H | CH₃ |
| 121 | Et | H | (CH₂)₃OH | CH₂CO₂Me | CO₂Me | H | CH₃ |
| 122 | Et | H | (CH₂)₃OH | CH(OH)CO₂Me | CO₂Me | H | CH₃ |
| 123 | Et | H | CH₂CH(OH)CH₂OH | CH₂CO₂Me | CO₂Me | H | CH₃ |
| 124 | Et | H | (CH₂)₂O(CH₂)₂OH | CH₂CO₂Me | CO₂Me | H | CH₃ |
| 125 | Et | H | (CH₂)₂O(CH₂)₂Ome | CH₂CO₂Me | CO₂Me | H | CH₃ |
| 126 | Et | H | (CH₂)₂O(CH₂)₂Oac | CH₂CO₂Me | CO₂Me | H | CH₃ |
| 127 | Et | H | (CH₂)₂O(CH₂)₂NH₂ | CH₂CO₂Me | CO₂Me | H | CH₃ |
| 128 | Et | H | (CH₂)₂O(CH₂)₂N(CH₃)₂ | CH₂CO₂Me | CO₂Me | H | CH₃ |
| 129 | Et | H | (CH₂)₂O(CH₂)₂N(CH₃)₃⁺I⁻ | CH₂CO₂Me | CO₂Me | H | CH₃ |
| 130 | Et | H | (CH₂)₃N(CH₂CH₂OH)₂ | CH₂CO₂Me | CO₂Me | H | CH₃ |
| 131 | Et | H | (CH₂)₃N(CH₂CH₂OMe)₂ | CH₂CO₂Me | CO₂Me | H | CH₃ |
| 132 | Et | H | (CH₂)₃N(CH₂CH₂OAc)₂ | CH₂CO₂Me | CO₂Me | H | CH₃ |
| 133 | Et | H | (CH₂)₂O(CH₂)₂O(CH₂)₂OH | CH₂CO₂Me | CO₂Me | H | CH₃ |
| 134 | Et | H | (CH₂)₂O(CH₂)₂O(CH₂)₂Ome | CH₂CO₂Me | CO₂Me | H | CH₃ |

TABLE 6
2-(2-Hydroxyethyl) chlorin e6 derivatives; M = H2

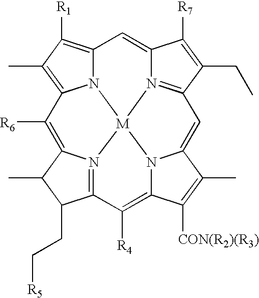

| Comp. No | R₁ | R₂ | R₃ | R₄ | R₅ | R₆ | R₇ |
|---|---|---|---|---|---|---|---|
| 135 | (CH₂)₂OH | H | CH₃ | CH₂CO₂Me | CO₂Me | H | CH₃ |
| 136 | (CH₂)₂OH | H | (CH₂)₄CH₃ | CH₂CO₂Me | CO₂Me | H | CH₃ |
| 137 | (CH₂)₂OH | H | (CH₂)₃OH | CH₂CO₂Me | CO₂Me | H | CH₃ |
| 138 | (CH₂)₂OH | H | CH₂CH(OH)CH₂OH | CH₂CO₂Me | CO₂Me | H | CH₃ |
| 139 | (CH₂)₂OH | H | (CH₂)₂O(CH₂)₂OH | CH₂CO₂Me | CO₂Me | H | CH₃ |
| 140 | (CH₂)₂OH | H | (CH₂)₂O(CH₂)₂Ome | CH₂CO₂Me | CO₂Me | H | CH₃ |
| 141 | (CH₂)₂OH | H | (CH₂)₂O(CH₂)₂Oac | CH₂CO₂Me | CO₂Me | H | CH₃ |
| 142 | (CH₂)₂OH | H | (CH₂)₂O(CH₂)₂NH₂ | CH₂CO₂Me | CO₂Me | H | CH₃ |
| 143 | (CH₂)₂OH | H | (CH₂)₂O(CH₂)₂N(CH₃)₂ | CH₂CO₂Me | CO₂Me | H | CH₃ |
| 144 | (CH₂)₂OH | H | (CH₂)₃N(CH₂CH₂OH)₂ | CH₂CO₂Me | CO₂Me | H | CH₃ |
| 145 | (CH₂)₂OH | H | (CH₂)₃N(CH₂CH₂OMe)₂ | CH₂CO₂Me | CO₂Me | H | CH₃ |
| 146 | (CH₂)₂OH | H | (CH₂)₃N(CH₂CH₂OAc)₂ | CH₂CO₂Me | CO₂Me | H | CH₃ |
| 147 | (CH₂)₂OH | H | (CH₂)₂O(CH₂)₂O(CH₂)₂OH | CH₂CO₂Me | CO₂Me | H | CH₃ |
| 148 | (CH₂)₂OH | H | (CH₂)₂O(CH₂)₂O(CH₂)₂OMe | CH₂CO₂Me | CO₂Me | H | CH₃ |

TABLE 7
2-(1-Hydroxyethyl) chlorin e6 derivatives; M = H2

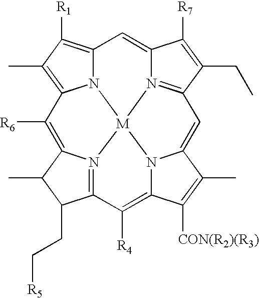

| Comp. No | R₁ | R₂ | R₃ | R₄ | R₅ | R₆ | R₇ |
|---|---|---|---|---|---|---|---|
| 149 | CH(OH)CH₃ | H | CH₃ | CH₂CO₂Me | CO₂Me | H | CH₃ |
| 150 | CH(OH)CH₃ | H | (CH₂)₄CH₃ | CH₂CO₂Me | CO₂Me | H | CH₃ |
| 151 | CH(OH)CH₃ | H | (CH₂)₂OH | CH₂CO₂Me | CO₂Me | H | CH₃ |
| 152 | CH(OH)CH₃ | H | (CH₂)₃OH | CH₂CO₂Me | CO₂Me | H | CH₃ |
| 153 | CH(OH)CH₃ | H | CH₂CH(OH)CH₂OH | CH₂CO₂Me | CO₂Me | H | CH₃ |
| 154 | CH(OH)CH₃ | H | (CH₂)₂O(CH₂)₂OH | CH₂CO₂Me | CO₂Me | H | CH₃ |
| 155 | CH(OH)CH₃ | H | (CH₂)₂O(CH₂)₂Ome | CH₂CO₂Me | CO₂Me | H | CH₃ |
| 156 | CH(OH)CH₃ | H | (CH₂)₂O(CH₂)₂Oac | CH₂CO₂Me | CO₂Me | H | CH₃ |
| 157 | CH(OH)CH₃ | H | (CH₂)₂O(CH₂)₂NH₂ | CH₂CO₂Me | CO₂Me | H | CH₃ |
| 158 | CH(OH)CH₃ | H | (CH₂)₂O(CH₂)₂N(CH₃)₂ | CH₂CO₂Me | CO₂Me | H | CH₃ |
| 159 | CH(OH)CH₃ | H | (CH₂)₃N(CH₂CH₂OH)₂ | CH₂CO₂Me | CO₂Me | H | CH₃ |
| 160 | CH(OH)CH₃ | H | (CH₂)₃N(CH₂CH₂OMe)₂ | CH₂CO₂Me | CO₂Me | H | CH₃ |
| 161 | CH(OH)CH₃ | H | (CH₂)₃N(CH₂CH₂OAc)₂ | CH₂CO₂Me | CO₂Me | H | CH₃ |
| 162 | CH(OH)CH₃ | H | (CH₂)₂O(CH₂)₂O(CH₂)₂OH | CH₂CO₂Me | CO₂Me | H | CH₃ |
| 163 | CH(OH)CH₃ | H | (CH₂)₂O(CH₂)₂O(CH₂)₂OMe | CH₂CO₂Me | CO₂Me | H | CH₃ |

TABLE 8
2-(1-Alkoxyethyl) chlorin e6 derivatives; M = H2

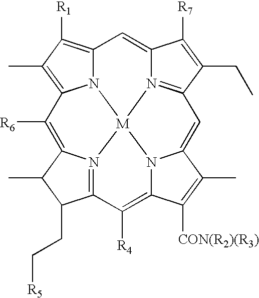

| Comp. No | R₁ | R₂ | R₃ | R₄ | R₅ | R₆ | R₇ |
|---|---|---|---|---|---|---|---|
| 164 | CH(OMe)CH₃ | H | CH₃ | CH₂CO₂Me | CO₂Me | H | CH₃ |
| 165 | CH(OMe)CH₃ | H | (CH₂)₃OH | CH₂CO₂Me | CO₂Me | H | CH₃ |
| 166 | CH(OMe)CH₃ | H | (CH₂)₂O(CH₂)₂OH | CH₂CO₂Me | CO₂Me | H | CH₃ |
| 167 | CH(OHeptyl)CH₃ | H | CH₃ | CH₂CO₂Me | CO₂Me | H | CH₃ |
| 168 | CH(OHeptyl)CH₃ | H | (CH₂)₃OH | CH₂CO₂Me | CO₂Me | H | CH₃ |
| 169 | CH(OHeptyl)CH₃ | H | (CH₂)₂O(CH₂)₂OH | CH₂CO₂Me | CO₂Me | H | CH₃ |
| 170 | CH(OHexyl)CH₃ | H | CH₃ | CH₂CO₂Me | CO₂Me | H | CH₃ |
| 171 | CH(OHexyl)CH₃ | H | (CH₂)₃OH | CH₂CO₂Me | CO₂Me | H | CH₃ |
| 172 | CH(OHexyl)CH₃ | H | (CH₂)₂O(CH₂)₂OH | CH₂CO₂Me | CO₂Me | H | CH₃ |
| 173 | CH(OHexyl)CH₃ | H | (CH₂)₂O(CH₂)₂N(CH₃)₂ | CH₂CO₂Me | CO₂Me | H | CH₃ |
| 174 | CH(OHexyl)CH₃ | H | (CH₂)₃N(CH₂CH₂OH)₂ | CH₂CO₂Me | CO₂Me | H | CH₃ |
| 175 | CH(OHexyl)CH₃ | H | (CH₂)₂O(CH₂)₂O(CH₂)₂OH | CH₂CO₂Me | CO₂Me | H | CH₃ |
| 176 | CH(OHexyl)CH₃ | H | (CH₂)₂O(CH₂)₂O(CH₂)₂OMe | CH₂CO₂Me | CO₂Me | H | CH₃ |

TABLE 9
2-(N,N-Dimethylaminoethyl)vinyl chlorin e6 derivatives; M = H2

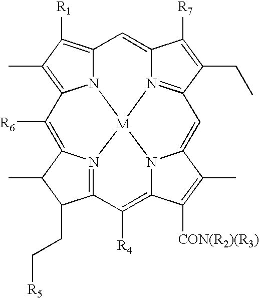

| Comp No | R₁ | R₂ | R₃ | R₄ | R₅ | R₆ | R₇ |
|---|---|---|---|---|---|---|---|
| 177 | CH═CHCH₂N(CH₃)₂ | H | CH₃ | CH₂CO₂Me | CO₂Me | H | CH₃ |
| 178 | CH═CHCH₂N(CH₃)₂ | H | (CH₂)₃OH | CH₂CO₂Me | CO₂Me | H | CH₃ |
| 179 | CH═CHCH₂N(CH₃)₂ | H | (CH₂)₂O(CH₂)₂OH | CH₂CO₂Me | CO₂Me | H | CH₃ |
| 180 | CH═CHCH₂N(CH₃)₂ | H | (CH₂)₂O(CH₂)₂N(CH₃)₂ | CH₂CO₂Me | CO₂Me | H | CH₃ |
| 181 | CH═CHCH₂N(CH₃)₂ | H | (CH₂)₃N(CH₂CH₂OH)₂ | CH₂CO₂Me | CO₂Me | H | CH₃ |
| 182 | CH═CHCH₂N(CH₃)₂ | H | (CH₂)₂O(CH₂)₂O(CH₂)₂OH | CH₂CO₂Me | CO₂Me | H | CH₃ |
| 183 | CH═CHCH₂N(CH₃)₂ | H | (CH₂)₂O(CH₂)₂O(CH₂)₂OMe | CH₂CO₂Me | CO₂Me | H | CH₃ |

TABLE 10
Vinyl chlorin e6 derivatives; M = Zn

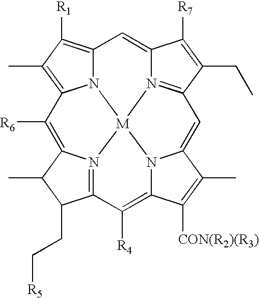

| Compd No | R₁ | R₂ | R₃ | R₄ | R₅ | R₆ | R₇ |
|---|---|---|---|---|---|---|---|
| 184 | V | H | (CH₂)₃OH | CH₂CO₂Me | CO₂Me | H | CH₃ |
| 185 | V | H | (CH₂)₃OH | CH₂CO₂H | CO₂H | H | CH₃ |
| 186 | V | H | (CH₂)₃OH | CH₂CO₂Na | CO₂Na | H | CH₃ |
| 187 | V | H | (CH₂)₃OH | CH(OH)CO₂Me | CO₂Me | H | CH₃ |
| 188 | V | H | (CH₂)₃Oac | CH₂CO₂Me | CO₂Me | H | CH₃ |
| 189 | V | H | (CH₂)₃OH | CH₂CO₂Me | CONHMe | H | CH₃ |
| 190 | V | H | (CH₂)₃OH | CH₂CH₂OH | CONHMe | H | CH₃ |
| 191 | V | H | (CH₂)₃OH | CH₂CH₂OH | CH₂OH | H | CH₃ |
| 192 | V | H | CH₂CH(OH)CH₂OH | CH₂CO₂Me | CO₂Me | H | CH₃ |
| 193 | V | H | (CH₂)₂O(CH₂)₂OH | CH₂CO₂Me | CO₂Me | H | CH₃ |
| 194 | V | H | (CH₂)₂O(CH₂)₂OH | CH₂CO₂H | CO₂H | H | CH₃ |
| 195 | V | H | (CH₂)₂O(CH₂)₂OH | CH₂CO₂Na | CO₂Na | H | CH₃ |
| 196 | V | H | (CH₂)₂O(CH₂)₂OH | CH(OH)CO₂Me | CO₂Me | H | CH₃ |
| 197 | V | H | (CH₂)₂O(CH₂)₂OH | CH₂CH₂OH | CH₂OH | H | CH₃ |
| 198 | V | H | (CH₂)₂O(CH₂)₂OMe | CH₂CO₂Me | CO₂Me | H | CH₃ |
| 199 | V | H | (CH₂)₂O(CH₂)₂OAc | CH₂CO₂Me | CO₂Me | H | CH₃ |
| 200 | V | H | (CH₂)₂O(CH₂)₂NH₂ | CH₂CO₂Me | CO₂Me | H | CH₃ |
| 201 | V | H | (CH₂)₂O(CH₂)₂N(CH₃)₂ | CH₂CO₂Me | CO₂Me | H | CH₃ |
| 202 | V | H | (CH₂)₂O(CH₂)₂N(CH₃)₃⁺I⁻ | CH₂CO₂Me | CO₂Me | H | CH₃ |
| 203 | V | H | (CH₂)₃N(CH₂CH₂OH)₂ | CH₂CO₂Me | CO₂Me | H | CH₃ |
| 204 | V | H | (CH₂)₃N(CH₂CH₂OMe)₂ | CH₂CO₂Me | CO₂Me | H | CH₃ |
| 205 | V | H | (CH₂)₃N(CH₂CH₂OAc)₂ | CH₂CO₂Me | CO₂Me | H | CH₃ |
| 206 | V | H | (CH₂)₂O(CH₂)₂O(CH₂)₂OH | CH₂CO₂Me | CO₂Me | H | CH₃ |
| 207 | V | H | (CH₂)₂O(CH₂)₂O(CH₂)₂OMe | CH₂CO₂Me | CO₂Me | H | CH₃ |
| 208 | V | H | (CH₂)₃OPO₃H₂ | CH₂CO₂Me | CO₂Me | H | CH₃ |
| 209 | V | H | (CH₂)₃OPO₃H | CH₂CO₂Me | CO₂Me | H | CH₃ |

TABLE 11

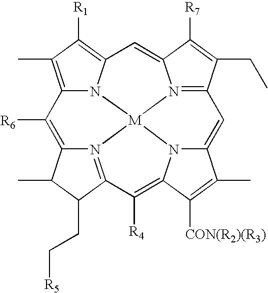

2-(1-Hydroxyethyl) chlorin e6 derivatives; M=Zn

| Comp | | | | | | | |
|---|---|---|---|---|---|---|---|
| No | R¹ | R² | R³ | R⁴ | R⁵ | R⁶ | R⁷ |
| 210 | CH(OH)CH₃ | H | CH₃ | CH₂CO₂Me | CO₂Me | H | CH₃ |
| 211 | CH(OH)CH₃ | H | (CH₂)₄CH₃ | CH₂CO₂Me | CO₂Me | H | CH₃ |
| 212 | CH(OH)CH₃ | H | (CH₂)₂OH | CH₂CO₂Me | CO₂Me | H | CH₃ |
| 213 | CH(OH)CH₃ | H | (CH₂)₃OH | CH₂CO₂Me | CO₂Me | H | CH₃ |
| 214 | CH(OH)CH₃ | H | CH₂CH(OH)CH₂OH | CH₂CO₂Me | CO₂Me | H | CH₃ |
| 215 | CH(OH)CH₃ | H | (CH₂)₂O(CH₂)₂OH | CH₂CO₂Me | CO₂Me | H | CH₃ |
| 216 | CH(OH)CH₃ | H | (CH₂)₂O(CH₂)₂OMe | CH₂CO₂Me | CO₂Me | H | CH₃ |
| 217 | CH(OH)CH₃ | H | (CH₂)₂O(CH₂)₂OAc | CH₂CO₂Me | CO₂Me | H | CH₃ |
| 218 | CH(OH)CH₃ | H | (CH₂)₂O(CH₂)₂NH₂ | CH₂CO₂Me | CO₂Me | H | CH₃ |
| 219 | CH(OH)CH₃ | H | (CH₂)₂O(CH₂)₂N(CH₃)₂ | CH₂CO₂Me | CO₂Me | H | CH₃ |
| 220 | CH(OH)CH₃ | H | (CH₂)₃N(CH₂CH₂OH)₂ | CH₂CO₂Me | CO₂Me | H | CH₃ |
| 221 | CH(OH)CH₃ | H | (CH₂)₃N(CH₂CH₂OMe)₂ | CH₂CO₂Me | CO₂Me | H | CH₃ |
| 222 | CH(OH)CH₃ | H | (CH₂)₃N(CH₂CH₂OAc)₂ | CH₂CO₂Me | CO₂Me | H | CH₃ |
| 223 | CH(OH)CH₃ | H | (CH₂)₂O(CH₂)₂O(CH₂)₂OH | CH₂CO₂Me | CO₂Me | H | CH₃ |
| 224 | CH(OH)CH₃ | H | (CH₂)₂O(CH₂)₂O(CH₂)₂OMe | CH₂CO₂Me | CO₂Me | H | CH₃ |

TABLE 12

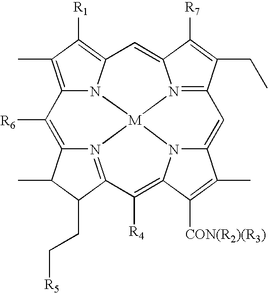

2-(N,N-dimethylaminomethyl)vinyl chlorin e6 derivatives; M=Zn

| Comp | | | | | | | |
|---|---|---|---|---|---|---|---|
| No | R¹ | R² | R³ | R⁴ | R⁵ | R⁶ | R⁷ |
| 225 | CH═CHCH₂N(CH₃)₂ | H | CH₃ | CH₂CO₂Me | CO₂Me | H | CH₃ |
| 226 | CH═CHCH₂N(CH₃)₂ | H | (CH₂)₃OH | CH₂CO₂Me | CO₂Me | H | CH₃ |
| 227 | CH═CHCH₂N(CH₃)₂ | H | (CH₂)₂O(CH₂)₂OH | CH₂CO₂Me | CO₂Me | H | CH₃ |
| 228 | CH═CHCH₂N(CH₃)₂ | H | (CH₂)₂O(CH₂)₂N(CH₃)₂ | CH₂CO₂Me | CO₂Me | H | CH₃ |
| 228 | CH═CHCH₂N(CH₃)₂ | H | (CH₂)₃N(CH₂CH₂OH)₂ | CH₂CO₂Me | CO₂Me | H | CH₃ |
| 230 | CH═CHCH₂N(CH₃)₂ | H | (CH₂)₂O(CH₂)₂O(CH₂)₂OH | CH₂CO₂Me | CO₂Me | H | CH₃ |
| 231 | CH═CHCH₂N(CH₃)₂ | H | (CH₂)₂O(CH₂)₂O(CH₂)₂OMe | CH₂CO₂Me | CO₂Me | H | CH₃ |

TABLE 13

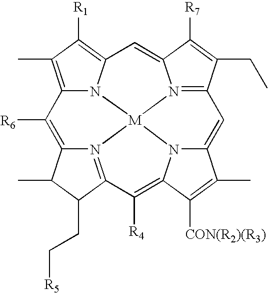

2-Acetyl chlorin e6 derivatives; M=Zn

| Comp | | | | | | | |
|---|---|---|---|---|---|---|---|
| No | R₁ | R₂ | R₃ | R₄ | R₅ | R₆ | R₇ |
| 232 | COCH₃ | H | CH₃ | CH₂CO₂Me | CO₂Me | H | CH₃ |
| 233 | COCH₃ | H | (CH₂)₂OH | CH₂CO₂Me | CO₂Me | H | CH₃ |
| 234 | COCH₃ | H | (CH₂)₅CH₃ | CH₂CO₂Me | CO₂Me | H | CH₃ |
| 235 | COCH₃ | H | (CH₂)₃OH | CH₂CO₂Me | CO₂Me | H | CH₃ |
| 236 | COCH₃ | H | (CH₂)₃OH | CH₂CO₂Me | CO₂Me | H | CH₃ |
| 237 | COCH₃ | H | CH₂CH(OH)CH₂OH | CH₂CO₂Me | CO₂Me | H | CH₃ |
| 238 | COCH₃ | H | (CH₂)₂O(CH₂)₂OH | CH₂CO₂Me | CO₂Me | H | CH₃ |
| 239 | COCH₃ | H | (CH₂)₂O(CH₂)₂OMe | CH₂CO₂Me | CO₂Me | H | CH₃ |
| 240 | COCH₃ | H | (CH₂)₂O(CH₂)₂OAc | CH₂CO₂Me | CO₂Me | H | CH₃ |
| 241 | COCH₃ | H | (CH₂)₂O(CH₂)₂NH₂ | CH₂CO₂Me | CO₂Me | H | CH₃ |
| 242 | COCH₃ | H | (CH₂)₂O(CH₂)₂N(CH₃)₂ | CH₂CO₂Me | CO₂Me | H | CH₃ |
| 243 | COCH₃ | H | (CH₂)₂O(CH₂)₂N(CH₃)₃⁺ I⁻ | CH₂CO₂Me | CO₂Me | H | CH₃ |
| 244 | COCH₃ | H | (CH₂)₃N(CH₂CH₂OH)₂ | CH₂CO₂Me | CO₂Me | H | CH₃ |
| 245 | COCH₃ | H | (CH₂)₃N(CH₂CH₂OMe)₂ | CH₂CO₂Me | CO₂Me | H | CH₃ |
| 246 | COCH₃ | H | (CH₂)₃N(CH₂CH₂OAc)₂ | CH₂CO₂Me | CO₂Me | H | CH₃ |
| 247 | COCH₃ | H | (CH₂)₂O(CH₂)₂O(CH₂)₂OH | CH₂CO₂Me | CO₂Me | H | CH₃ |
| 248 | COCH₃ | H | (CH₂)₂O(CH₂)₂O(CH₂)₂OMe | CH₂CO₂Me | CO₂Me | H | CH₃ |

TABLE 14

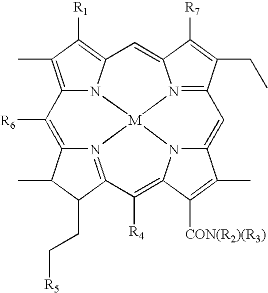

2-(Hydroxymethyl) chlorin e6 derivatives; M=Zn

| Comp | | | | | | | |
|---|---|---|---|---|---|---|---|
| No | R₁ | R₂ | R₃ | R₄ | R₅ | R₆ | R₇ |
| 249 | CH₂OH | H | CH₃ | CH₂CO₃Me | CO₂Me | H | CH₃ |
| 250 | CH₂OH | H | (CH₂)₂OH | CH₂CO₃Me | CO₂Me | H | CH₃ |
| 251 | CH₂OH | H | (CH₂)₃OH | CH₂CO₃Me | CO₂Me | H | CH₃ |
| 252 | CH₂OH | H | (CH₂)₃OH | CH₂CO₃Me | CONHMe | H | CH₃ |
| 253 | CH₂OH | H | CH₂CH(OH)CH₂OH | CH₂CO₃Me | CO₂Me | H | CH₃ |
| 254 | CH₂OH | H | (CH₂)₂O(CH₂)₂OH | CH₂CO₃Me | CO₂Me | H | CH₃ |
| 255 | CH₂OH | H | (CH₂)₂O(CH₂)₂OMe | CH₂CO₃Me | CO₂Me | H | CH₃ |
| 256 | CH₂OH | H | (CH₂)₂O(CH₂)₂OAc | CH₂CO₃Me | CO₂Me | H | CH₃ |
| 257 | CH₂OH | H | (CH₂)₂O(CH₂)₂NH₂ | CH₂CO₃Me | CO₂Me | H | CH₃ |
| 258 | CH₂OH | H | (CH₂)₂O(CH₂)₂N(CH₃)₂ | CH₂CO₃Me | CO₂Me | H | CH₃ |
| 259 | CH₂OH | H | (CH₂)₂O(CH₂)₂N(CH₃)₃⁺ I⁻ | CH₂CO₃Me | CO₂Me | H | CH₃ |
| 260 | CH₂OH | H | (CH₂)₃N(CH₂CH₂OH)₂ | CH₂CO₃Me | CO₂Me | H | CH₃ |
| 261 | CH₂OH | H | (CH₂)₃N(CH₂CH₂OMe)₂ | CH₂CO₃Me | CO₂Me | H | CH₃ |
| 262 | CH₂OH | H | (CH₂)₃N(CH₂CH₂OAc)₂ | CH₂CO₃Me | CO₂Me | H | CH₃ |
| 263 | CH₂OH | H | (CH₂)₂O(CH₂)₂O(CH₂)₂OH | CH₂CO₃Me | CO₂Me | H | CH₃ |
| 264 | CH₂OH | H | (CH₂)₂O(CH₂)₂O(CH₂)₂OMe | CH₂CO₃Me | CO₂Me | H | CH₃ |

TABLE 15

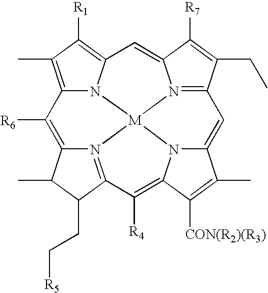

Gallium chlorin e6 derivatives; M=GaCl

| Comp | | | | | | | |
|---|---|---|---|---|---|---|---|
| No | R₁ | R₂ | R₃ | R₄ | R₅ | R₆ | R₇ |
| 265 | V | H | CH₃ | CH₂CO₂Me | CO₂Me | H | CH₃ |
| 266 | V | H | (CH₂)₄CH₃ | CH₂CO₂Me | CO₂Me | H | CH₃ |
| 267 | V | H | (CH₂)₃OH | CH₂CO₂Me | CO₂Me | H | CH₃ |
| 268 | Et | H | CH₃ | CH₂CO₂Me | CO₂Me | H | CH₃ |
| 269 | Et | H | (CH₂)₄CH₃ | CH₂CO₂Me | CO₂Me | H | CH₃ |
| 270 | Et | H | (CH₂)₃OH | CH₂CO₂Me | CO₂Me | H | CH₃ |
| 271 | COCH₃ | H | CH₃ | CH₂CO₂Me | CO₂Me | H | CH₃ |
| 272 | COCH₃ | H | (CH₂)₄CH₃ | CH₂CO₂Me | CO₂Me | H | CH₃ |
| 273 | COCH₃ | H | (CH₂)₃OH | CH₂CO₂Me | CO₂Me | H | CH₃ |
| 274 | V | H | (CH₂)₂O(CH₂)₂OH | CH₂CO₂Me | CO₂Me | H | CH₃ |
| 275 | COCH₃ | H | (CH₂)₂O(CH₂)₂OH | CH₂CO₂Me | CO₂Me | H | CH₃ |
| 276 | Et | H | (CH₂)₂O(CH₂)₂OH | CH₂CO₂Me | CO₂Me | H | CH₃ |

The scope of the present invention is not limited to the compounds provided in Tables 1-15. As shown above, any pheophorbide or chlorin e6 molecule may be modified according to the invention to form the desired photoactive compounds with widely differing functionality. Examples of such. functionality at positions R₁, R₄, R₅, R₆, R₇ and R₈ (FIGS. I and II) are described in the literature (for example, see “Porphyrins and Metalloporphyrins” ed. K. Smith, Elsevier, 1975, N.Y. and “The Porphyrins”, Ed D. Dolphin, Vol I-V, Academic Press, 1978; “The Porphyrin Handbook”, Ed. K. Kadish, K. M. Smith, R. Guilard, Academic Press, 1999). These compounds contain various and ranging substituents on the β-pyrrole positions or meso-positions of the chlorin ring. Such functionality once incorporated onto the ring structure, may be further modified by attachment to other molecules (for example antibodies and the like) before or after ring opening of the pheophorbide with the desired amine has occurred.

Examples of such functionality at positions R₁, R₄, R₅, R₆, R₇ and R₈ include functional groups having a molecular weight less than about 100,000 daltons and can be a biologically active group or organic in nature. Examples include, but are not limited to: (1) hydrogen; (2) halogen, such as fluoro, chloro, iodo and bromo (3) lower alkyl, such as methyl, ethyl, CH(CH₃)₂, n-propyl, butyl, hexyl, heptyl, octyl, t-butyl, n-pentyl and the like groups; (4) lower alkoxy, such as methoxy, ethoxy, isopropoxy, n-butoxy, n-pentoxy and the like; (5) hydroxy; (6) carboxylic acid or acid salts, such as —CH₂COOH, —CH₂COONa, —CH₂CH₂COOH, —CH₂CH₂COONa, —CH₂CH₂CH(Br)COOH, —CH₂CH₂CH(CH₃)COOH, —CH₂CH(Br)COOH, —CH₂CH(CH₃)COOH, —CH(Cl)CH₂CH(CH₃)COOH, —CH₂CH₂C(CH₃)₂COOH, —CH₂CH₂C(CH₃)₂COOK, —CH₂CH₂CH₂CH₂COOH, C(CH₃)₂COOH, CH(Cl)₂COOH and the like; (7) carboxylic acid esters, such as —CH₂CH₂COOCH₃, —CH₂CH₂COOCH₂CH₃, —CH₂CH(CH₃)COOCH₂CH₃, —CH₂CH₂COOCH₂CH₂CH₃, —CH₂CH₂CH₂COOCH₂CH₂CH₃, —CH₂CH(CH₃)COOCH₂CH₃, —CH₂CH₂COOCH₂CH₂OH, —CH₂CH₂COOCH₂CH₂N(CH₃)₂ and the like, particularly halogenated alkyl esters; (8) sulfonic acid or acid salts, for example, group I and group II salts, ammonium salts, and organic cation salts such as quaternary ammonium salts; (9) sulfonylamides such as —SO₂NH(alkyl), —SO₂N(alkyl)₂, —SO₂NH(alkyl-OH), —SO₂N(alkyl-OH)₂, —SO₂NH(alkyl)-N(alkyl)₂, —SO₂N(alkyl-N(alkyl)₂)₂, SO₂(NH(alkyl)-N(alkyl)₃⁺Z⁻) and the like, wherein Z⁻ is a counterion, —SO₂NHCH₂CO₂H, substituted and unsubstituted benzene sulfonamides and sulfonylamides of aminoacids and the like; (10) sulfonic acid esters, such as SO₃(alkyl), SO₃(alkyl-OH), SO₃(alkyl-N(alkyl)₂), SO₃(alkyl-N(alkyl)₃⁺Z⁻) and the like, wherein Z⁻ is a counterion, SO₃CH₂CO₂H, and the like; (11) amino, such as unsubstituted or substituted primary amino, methylamino, ethylamino, n-propylamino, isopropylamino, butylamino, sec-butylamino, dimethylamino, trimethylammonium, diethylamino, triethylammonium, di-n-propylamino, methylethylamino, dimethyl-sec-butylammonium, 2-aminoethoxy, ethylenediamino, cyclohexylamino, benzylamino, phenylethylamino, anilino, N-methylanilino, N,N-dimethylanilino, N-methyl-N-ethylanilino, 3,5-dibromo-4-anilino, p-toluidino, diphenylamino, 4,4′-dinitrodiphenylamino and the like; (12) cyano; (13) nitro; (14) a biologically active group; (15) amides, such as —CH₂CH₂CONHCH₃, —CH₂CH₂CONHCH₂CH₃, —CH₂CH₂CON(CH₃)₂, —CH₂CH₂CON(CH₂CH₃)₂, —CH₂CONHCH₃, —CH₂CONHCH₂CH₃, —CH₂CON(CH₃)₂, —CH₂CON(CH₂CH₃)₂, —CH₂CH₂CONHSO₂CH₃; (16) iminium salts, for example CH═N(CH₃)₂⁺Z⁻ and the like, wherein Z⁻ is a counterion; (17) boron-containing complexes; (18) carbon cage complexes (e.g., C20 and the like); (19) polyfunctional carboxylic acid groups and their metal cluster complexes, for example metal complexes of polyfunctional carboxylic acid moieties such as of EDTA, DTPA and the like, crown ethers, cyclams, cyclens, and the like; (20) other porphyrin, chlorin, bacteriochlorin, isobacteriochlorin, azaporphyrin, tetraazaporphyrin, phthalocyanine, naphthalocyanine, texaphyrins, tetrapyrrolic macrocycles or dye molecules and the like; (21) alkynyl, including alkyl, aryl, acid and heteroatom substituted alkynes; (22) leaving or protecting groups; (23) aromatic ring systems (aryl) either substituted or not, such as phenyls, napthalenes, anthracenes, benzopyrenes, quinolines, benzoquinolines, benzoperylene, benzofluorenes, fluorenes, benzofurazans, benzodiphenylenes, benzofluoranthenes, benzanthracenes, benzacephenanthrylenes, bathophenanthrolines, indans, benzoquinolines, quinolines, pyrazines, quinolines, quinazoles, quinoxalines, imidazopyridines, indenes, indolines, thiazolines, bezopyrimidines, pyrimidines, benzimidazole, triazolopyrimidines, pyrazoles, tryptophans, phenanthrolines, benzooxadiazoles, benzoselenadiazole, benzocoumarins, chalcones, fluoranthenes, pyridoindoles, pentacenes, perylenes, phenatholines, phenazines, phenoxazines, phenoxathiins, phenothiazines, pyrroles, thiophenes, or heteroaromatics containing 5, 6, 7, or 8 membered ring systems; 24) —NCS groups; and 25) polyether groups, hydroxylated polyether groups, or any other substituent that increases the hydrophilic, amphiphilic or lipophilic nature or stability of the compounds. It is recognized that such groups can affect the biological activity of the compounds in vivo.

Particularly preferred are chlorin e6 analogs that possess at R₂ and R₃, groups selected from H, alkyl, cycloalkyl, aryl, (CH₂)_{w}OH, (CH₂)ₙO-alkyl, (CH₂)ₙOCOCH₃, CH₂CH(OH)CH₂OH, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, ((CH₂)ₙO)ₘ(CH₂)_{Q}OH, ((CH₂)ₙO)ₘ(CH₂O)_{Q}COCH₃, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN((CH₂)ₘN(CH₃)₂)₂, (CH₂)ₙN((CH₂)ₘN(CH₃)₃⁺A)₂, ((CH₂)ₙO)ₘ(CH₂)_{Q}OH, ((CH₂)ₙO)ₘ(CH₂O)_{Q}COCH₃, (CH₂)ₙO-haloalkyl, (CH₂)ₙN((CH₂)ₘO-alkyl)₂, (CH₂)ₙN((CH₂)ₘO-alkylether)₂, a mono-, di or polyhydroxyalkyl residue, CH₂CH(OAc)CH₂OAc, an alkylphosphate residue, an alkylsulfonic acid residue, an alkylsulfonic ester or alkylsulfonic amide reside, an alkylmorpholino residue, an alkylheterocyclic residue, an alkylthiol residue, a mono-, di-, or polyhydroxyaryl residue, a mono-, di-, or polyetheralkyl residue, a mono-, di-, or polyetheraryl residue, polyhydroxylated polyether residues and where Q, n and m may be the same or different and are integers ranging from 0 to 10,000, w is 1 or an integer ranging from 3 to 10,000, and A is a charge balancing ion;

In addition to the groups described, peripheral functionalization with biologically active groups is another preferred embodiment. The term “biologically active group” can be any group that selectively promotes the accumulation, elimination, binding rate, or tightness of binding in a particular biological environment. For example, one category of biologically active groups is the substituents derived from sugars, specifically: (1) aldoses such as glyceraldehyde, erythrose, threose, ribose, arabinose, xylose, lyxose, allose, altrose, glucose, mannose, gulose, idose, galactose, and talose; (2) ketoses such as hydroxyacetone, erythrulose, rebulose, xylulose, psicose, fructose, sorbose, and tagatose; (3) pyranoses such as glucopyranose; (4) furanoses such as fructo-furanose; (5) O-acyl derivatives such as penta-O-acetyl-α-glucose; (6) O-methyl derivatives such as methyl α-glucoside, methyl β-glucoside, methyl α-glucopyranoside, and methyl-2,3,4,6-tetra-O-methyl-glucopyranoside; (7) phenylosazones such as glucose phenylosazone; (8) sugar alcohols such as sorbitol, mannitol, glycerol, and myo-inositol; (9) sugar acids such as gluconic acid, glucaric acid and glucuronic acid, δ-gluconolactone, δ-glucuronolactone, ascorbic acid, and dehydroascorbic acid; (10) phosphoric acid esters such as α-glucose 1-phosphoric acid, α-glucose 6-phosphoric acid, α-fructose 1,6-diphosphoric acid, and α-fructose 6-phosphoric acid; (11) deoxy sugars such as 2-deoxy-ribose, rhammose (deoxy-mannose), and fructose (6-deoxy-galactose); (12) amino sugars such as glucosamine and galactosamine; muramic acid and neurarninic acid; (13) disaccharides such as maltose, sucrose and trehalose; (14) trisaccharides such as raffinose (fructose, glucose, galactose) and melezitose (glucose, fructose, glucose); (15) polysaccharides (glycans) such as glucans and mannans; and (16) storage polysaccharides such as α-amylose, amylopectin, dextrins, and dextrans.

Amino acid derivatives are also useful biologically active substituents, such as those derived from valine, leucine, isoleucine, threonine, methionine, phenylalanine, tryptophan, alanine, arginine, aspartic acid, cystine, cysteine, glutamic acid, glycine, histidine, proline, serine, tyrosine, asparagine and glutamine. Also useful are peptides, particularly those known to have affinity for specific receptors, for example, oxytocin, vasopressin, bradykinin, LHRH, thrombin and the like.

Another useful group of biologically active substituents are those derived from nucleosides, for example, ribonucleosides such as adenosine, guanosine, cytidine, and uridine; and 2′-deoxyribonucleosides, such as 2′-deoxyadenosine, 2′-deoxyguanosine, 2′-deoxycytidine, and 2′-deoxythymidine.

Another category of biologically active groups that is particularly useful is any ligand that is specific for a particular biological receptor. The term “ligand specific for a biological receptor” refers to a moiety that binds a receptor at cell surfaces, and thus contains contours and charge patterns that are complementary to those of the biological receptor. The ligand is not the receptor itself, but a substance complementary to it. It is well understood that a wide variety of cell types have specific receptors designed to bind hormones, growth factors, or neurotransmitters. However, while these embodiments of ligands specific for receptors are known and understood, the phrase “ligand specific for a biological receptor”, as used herein, refers to any substance, natural or synthetic, that binds specifically to a receptor.

Examples of such ligands include: (1) the steroid hormones, such as progesterone, estrogens, androgens, and the adrenal cortical hormones; (2) growth factors, such as epidermal growth factor, nerve growth factor, fibroblast growth factor, and the like; (3) other protein hormones, such as human growth hormone, parathyroid hormone, and the like; (4) neurotransmitters, such as acetylcholine, serotonin, dopamine, and the like; and (5) antibodies. Any analog of these substances that also succeeds in binding to a biological receptor is also included within the invention.

Particularly useful examples of substituents tending to increase the amphiphilic nature of the compounds include, but are not limited to: (1) short or long chain alcohols, such as, for example, —C₁₂H₂₄—OH; (2) fatty acids and their salts, such as, for example, the sodium salt of the long-chain fatty acid oleic acid; (3) phosphoglycerides, such as, for example, phosphatidic acid, phosphatidyl ethanolamine, phosphatidyl choline, phosphatidyl serine, phosphatidyl inositol, phosphatidyl glycerol, phosphatidyl 3′-O-alanyl glycerol, cardiolipin, or phosphatidyl choline; (4) sphingolipids, such as, for example, sphingomyelin; and (5) glycolipids, such as, for example, glycosyldiacylglycerols, cerebrosides, sulfate esters of cerebrosides or gangliosides. It would be known to those skilled in the art what other substituents, or combinations of the substituents described, would be suitable for use in the invention.

The compounds of the present invention, or their pharmaceutically acceptable salts, solvates, prodrugs, or metabolites, can be administered to the host in a variety of forms adapted to the chosen route of administration, e.g., orally, intravenously, topically, intramuscularly, subcutaneously or via retrobulbar intravitreal injection.

The active compound may be orally administered, for example, with an inert diluent or with an assimilable edible carrier, it may be enclosed in hard or soft shell gelatin capsule, compressed into tablets, or incorporated directly with food. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least about 0.1% of active compound. The percentage of the compositions and preparations may, of course, be varied and may, for example, conveniently be between about 2 to about 60% of the weight of the administered product. The amount of active compound in such therapeutically useful compositions can be selected so that a suitable dosage will be obtained. Preferred compositions or preparations according to the present invention are prepared so that an oral dosage unit form contains between about 50 and 300 mg of active compound.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; a sweetening agent such as sucrose, lactose or saccharin; or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations.

The active compound may also be administered parenterally or intraperitoneally. Solutions of the active compound as a free base or pharmacologically acceptable salt can be prepared in water suitably mixed with a surfactant such as hydroxypropylcellulose. Dispersions can also be prepared in glycerol, liquid polyethylene glycols, and mixtures thereof, and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions, dispersions, or liposomal or emulsion formulations. In all cases the form must be sterile and should be fluid to enable administration by a syringe. The form must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersions and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use of agents delaying absorption, for example, aluminum monostearate and gelatin.

Sterile injectable solutions are prepared by incorporating the active compound in the required amount in the appropriate solvent with various other ingredients enumerated above, as required, followed by filtered sterilization. Generally, dispersions are prepared by incorporating the various sterilized active ingredients into a sterile vehicle that contains the basic dispersion medium and the required additional ingredients from those enumerated above. In the case of sterile powders for the preparation of sterile injectable solutions, the preferred methods of preparation are vacuum drying and freeze-drying, which yield a powder of the active ingredient plus any additional desired ingredient from previously sterile-filtered solutions thereof.

The new compounds of the invention may also be applied directly to tumors in the host whether internal or external, in topical compositions. Exemplary compositions include solutions of the new compounds in solvents, particularly aqueous solvents, most preferably water. Alternatively, for topical application particularly to skin tumors or psoriasis, the compounds of the invention may be dispersed in cream or salve formulations commonly used for this purpose (such as liposomes, ointments, gels, hydrogels, cremes and oils) or may be provided in the form of spray solutions or suspensions that may include a propellant usually employed in aerosol preparations.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Any conventional media or agent that is compatible with the active ingredient can be used in the therapeutic compositions of the invention. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated. Each unit contains a predetermined quantity of active material calculated to produce the desired therapeutic effect in association with the required pharmaceutical carrier. The specifications for the novel dosage unit forms of the invention are dictated by and directly dependent on (a) the unique characteristics of the active material and the particular therapeutic effect to be achieved, and (b) the limitations inherent in the art of compounding active materials for the treatment of cardiovascular diseases, diseases of the skin, and cancers in living subjects.

The present invention provides a method of treating live cells, which includes, but is not limited to, animals such as humans and other mammals. The “mammals” also include farm animals, such as cows, hogs and sheep, as well as pet or sport animals, such as horses, dogs and cats. The dosage of the pharmaceutical compositions of the invention is dependent on the method of administration, the patient's age, severity of the disease, and the like.

The compounds of the invention may be taken parentally or orally, generally being administered intravascularly, subcutaneously, or intramuscularly or interperitoneally. The subject compounds may also be administered by inhalation, perivascular delivery, pericardial delivery (into perivascular sac), periadvential delivery (e.g., using a hydrogel wrap around the vessel), endovascular balloon catheters with micropores, channels, transmural injection ports, and the like.

For local catheter-based delivery of the compounds of the invention, an infusate can be placed and pressurized to facilitate intramural and transmural penetration into the target vessel. Local delivery can also be enhanced by other mechanical and electrical means. The depth of the penetration of the subject compounds by this local delivery method is a function of pressure in the perfused segment and the dwell time. Although little attention has been paid to the quantitative characteristics of the compounds of the invention in this setting, deposition of the substance should obey the principles governing transmural convection and diffusion.

Delivery of the compounds of the invention may also be via antibody-drug conjugates, internalizing antibodies or antibody fragments conjugated to compounds into cells using endocytosis. The subject compounds may also be impregnated into stent struts for local delivery. The route of administration of these pharmaceutical preparations is not critical, but may be selected according to the dosage form, the patient's age, the severity of the disease to be treated and other factors.

The compounds of the invention may find use in conjunction with other interventions, diagnostics and therapies, where lower levels of other therapies having significant side effects may be used effectively to reduce the detrimental side effects. Adjunctive interventions may include, but are not limited to: balloon angioplasty, invasive and non-invasive surgical procedures, stent deployment, cutting balloons, embolic protection devices, rotational and directional atherectomy, eximer lasers and the like.

Adjunctive therapies may include, but are not limited to: radiation therapy, chemotherapy, anti-platelet agents, vasodilators, antihypertensives, anti-arrhythmics, hyperthermia, cryotherapy, magnetic force, viral and non-viral gene therapy, pharmacogenetic therapy, antibodies, vaccines, glycoprotein IIb/IIIa Inhibitors, growth factors, peptides, DNA delivery, nucleic acids, anticancer drugs, steroid hormones, anti-inflammatories, proteins, anti-apoptotic therapies, anti-sense agents, immunotoxins, immunomodulators, antibody-drug conjugates, anti-proliferative therapies, drug eluting stents containing pharmacologically active agents, transplant products and processes, prostaglandins and catheter based devices to detect vulnerable plaques, hormone products, chelating agents, diuretics, cardiac glycosides, bronchodilators, antibiotics, antivirals, antitoxins, cyclosporins, thrombolytic agents, interferons, blood products such as parental iron and hemin, anti-fungal agents, antianginals, anticoagulants, analgesics, narcotics, neuromuscular blockers, sedatives, bacterial vaccines, viral vaccines, DNA or RNA of natural or synthetic origin including recombinent RNA and DNA, cytokines and their antagonists/inhibitors, chemokines and their antagonists/inhibitors.

Adjunctive diagnostics may include, but are not limited to: intra-vascular ultrasound imaging, angiography, quantitative vessel measurements and the use of radiological contrast agents, hormone products, chelating agents, diuretics, cardiac glycosides, bronchodilators, antibiotics, antivirals, antitoxins, cyclosporins, thrombolytic agents, interferons, blood products such as parental iron and hemin, anti-fungal agents, antianginals, anticoagulants, analgesics, narcotics, neuromuscular blockers, sedatives, bacterial vaccines, viral vaccines, DNA or RNA of natural or synthetic origin including recombinent RNA and DNA, cytokines and their antagonists/inhibitors, and chemokines and their antagonists/inhibitors.

The method of the invention can include administration of the particular chlorin photosensitizing compound prior to, concomitant with, or subsequent to a particular adjunctive therapy. A particular regimen is employed for administration, where a single bolus or plurality of doses may be administered to the patient. The particular protocol will depend upon the nature of the tissue to be treated, the particular compound that is employed and the severity of the disease. Target tissue structure and function, carriers, endocytosis, and other cellular transport mechanisms may be important for particular compounds when determining the specific mode of delivery. Administration will preferably be within about 3 days prior to vessel activation with an energy source, and desirably will be the same day as the treatment of the target vessel.

The compounds of the invention may be formulated in a variety of ways, depending upon the manner of the administration, the particular compound, the number of administrations, other drugs, the presence of other active components and the like. The formulation will generally be in a physiologically acceptable form, using various carriers, such as water, deionized water, phosphate buffered saline, aqueous ethanol, vegetable oils, liposomes, emulsions, inclusion complex (cyclodextrans). In some instances the formulation may be formulated as a slow release formulation, where the subject compounds may be encapsulated in a wide variety of carriers, may be administered as capsules, or as a prodrug.

Thus, for instance, when they are provided in the form of tablets, pills, solutions, suspensions, emulsions, granules or capsules, the preparations are typically administered orally. Injectable solutions are usually administered intravenously, either alone or in a mixture with conventional fluids for parenteral infusion containing sugars, amino acids, saline and the like. Local administration may be by injection at the site of the living cells, by insertion or attachment of a solid carrier at the site, or by direct, topical application of a viscous liquid. Specifically, when necessary, solutions may be administered as is by the intramuscular, intradermal, subcutaneous or intraperitoneal route. Suppositories are administered rectaily, and eye drops are instilled into the eye. The delivery of the compounds of the invention to living cells may be enhanced by the use of controlled-release compositions.

The compounds of the invention may also be applied externally by introducing them into a spray together with a suitable propellant and, if desired, a solvent, as a fine powder together with a suitable filler, and as a cream in combination with known auxiliaries. Furthermore, they may be used in the form of suppositories. They may also contain the required auxiliaries, such as fillers, lubricants, preservatives and emulsifying agents prepared by any method known in the art.

The pharmaceutical compositions of the invention may also contain a pharmaceutically acceptable carrier, such as saline, buffered saline, 5% dextrose in water, borate-buffered saline containing trace metal, carboxymethyl cellulose, vegetable oil, DMSO, ethanol, and the like. Formulations may further include one or more excipients, preservatives, antioxidants, solubilizers, buffering agents, albumin to prevent protein loss on vial surfaces, lubricants, fillers, stabilizers, and the like. Methods of formulation are well-known in the art and are disclosed, for example, in Remington's Pharmaceutical Sciences, Mack Publishing Co., Easton, Pa. (Gennaro, ed. 1990).

In preparing fluids for injection, the solutions or suspensions are preferably sterilized and isotonic with blood. For preparing such dosage forms, all the diluents in conventional use in this field can be employed. Thus, for example, water, ethyl alcohol, propylene glycol, ethoxylated isostearyl alcohol, polyoxylated isostearyl alcohol, liposomes and polyoxyethylene sorbitan fatty acid esters may be used. In this case, the pharmaceutical preparations may contain sodium chloride, glucose, lactose or glycerol in an amount sufficient to give isotonic solutions. It is also possible to add conventional solubilizing agents, buffers, soothing agents or local anesthetics, etc. Further, when appropriate, the pharmaceutical preparations may contain coloring materials, preservatives, perfumes, flavoring agents, sweetening agents and the like.

The proportion of the active ingredient compound in the pharmaceutical preparations of the invention is not critical, but may suitably be selected from a wide range. Generally, however, the proportion is preferably within the range of from about 0.01 to about 70% by weight.

Depending upon the manner of administration, the frequency of administration, as well the nature and the degree of the biological activity, the dosage will generally be in the range of about 0.01 to about 100 mg/kg. When administered parenterally, the total amount of the compound administered per day will generally be in the range of about 0.1 to 50 mg/kg/day, more usually in the range of about 0.25 to 25 mg/kg/day. This dose may be in a single bolus or divided up to be administered in portions to provide the desired level of the subject compound in the mammal.

Light doses appropriate to activate the compounds of the invention can be administered externally or internally to the target tissue. A particular regimen is employed for light administration, where a single dose or plurality of dosimetries may be administered to the patient. The particular protocol will depend upon the nature of the tissue to be treated, the particular compound that is employed and the severity of the disease. Light delivery devices can be, for example, in the form of a balloon catheter, bare tip diffuser and the like for endovascular delivery of light to blood-carrying vessels.

As used herein, the term light is to be considered in its broadest sense, encompassing all electromagnetic radiation. Light suitable for use in activating the compounds of the invention will typically be produced by, for example, arc lamps, LEDs or lasers at a certain frequency in the visible spectrum or near infrared for typical PDT treatments. In particular, wavelengths between 400 nm and 900 nm, corresponding to laser diode activation, may also be used. Additionally dual photon excitation may also be used.

Although described primarily with reference to presently preferred embodiments, one skilled in the art should recognize that various modifications and improvements are within the scope of this invention. It will be clearly understood that the invention in its general aspects is not limited to the specific details referred to herein.

### Definitions

As used in the present application, the following definitions apply:

The term “alkyl” as used herein refers to substituted or unsubstituted, straight or branched chain groups, preferably having one to twenty, more preferably having one to six, and most preferably having from one to four carbon atoms. The term “C₁-C₂₀ alkyl” represents a straight or branched alkyl chain having from one to twenty carbon atoms. Exemplary C₁-C₂₀ alkyl groups include methyl, ethyl, n-propyl, isopropyl, butyl, isobutyl, sec-butyl, t-butyl, pentyl, neo-pentyl, hexyl, isohexyl, and the like. The term “C₁-C₂₀ alkyl” includes within its definition the term “C₁-C₄ alkyl.” Such alkyl groups may themselves be ethers or thioethers, or aminoethers or dendrimers.

The term “cycloalkyl” represents a substituted or unsubstituted, saturated or partially saturated, mono- or poly-carbocyclic ring, preferably having 5-14 ring carbon atoms. Exemplary cycloalkyls include monocyclic rings having from 3-7, preferably 3-6, carbon atoms, such as cyclopropyl, cyclobutyl, cyclopentyl, cyclohexyl, cycloheptyl and the like. An exemplary cycloalkyl is a C₅-C₇ cycloalkyl, which is a saturated hydrocarbon ring structure containing from five to seven carbon atoms.

The term “aryl” as used herein refers to an aromatic, monovalent monocyclic, bicyclic, or tricyclic radical containing 6, 10, 14, or 18 carbon ring atoms, which may be unsubstituted or substituted, and to which may be fused one or more cycloalkyl groups, heterocycloalkyl groups, or heteroaryl groups, which themselves may be unsubstituted or substituted by one or more suitable substituents. Illustrative examples of aryl groups include, but are not limited to, phenyl, napthalenes, anthracenes, benzopyrenes, quinolines, benzoquinolines, benzoperylene, benzofluorenes, fluorenes, benzofurazans, benzodiphenylenes, benzofluoranthenes, benzanthracenes, benzacephenanthrylenes, bathophenanthrolines, indans, benzoquinolines, quinolines, pyrazines, quinolines, quinazoles, quinoxalines, imidazopyridines, indenes, indolines, thiazolines, benzopyrimidines, pyrimidines, benzimidazole, triazolopyrimidines, pyrazoles, tryptophans, phenanthrolines, benzooxadiazoles, benzoselenadiazole, benzocoumarins, chalcones, fluoranthenes, pyridoindoles, pentacenes, perylenes, phenatholines, phenazines, phenoxazines, phenoxathins, phenothiazines and the like.

The term “halogen” represents chlorine, fluorine, bromine or iodine. The term “halocarbon” or “haloalkyl” represents one or more halogens bonded to one or more carbon bearing groups. The term “heterohaloalkyl” represents, for example, halogenated alkylethers, halogenated alkyl amines, halogenated alkyl esters, halogenated alkyl amides, halogenated alkyl thioesters, halogenated alkyl thiols, where N, S, O, P atoms are present in the haloalkylated structure. The term heteroalkyl represents, for example, ethers, alkylamines, alkylated thiols and alkylate phosphorus containing groups.

The term “carbocycle” represents a substituted or unsubstituted aromatic or a saturated or a partially saturated 5-14 membered monocyclic or polycyclic ring, such as a 5- to 7-membered monocyclic or 7- to 10-membered bicyclic ring, wherein all the ring members are carbon atoms.

The term “electron withdrawing group” is intended to mean a chemical group containing an electronegative element such as halogen, sulfur, nitrogen or oxygen.

A “heterocycloalkyl group” is intended to mean a non-aromatic, monovalent monocyclic, bicyclic, or tricyclic radical, which is saturated or unsaturated, containing 3 to 18 ring atoms, and which includes 1 to 5 heteroatoms selected from nitrogen, oxygen and sulfur, wherein the radical is unsubstituted or substituted, and to which may be fused one or more cycloalkyl groups, aryl groups, or heteroaryl groups, which themselves may be unsubstituted or substituted. Illustrative examples of heterocycloalkyl groups include, but are not limited to, azetidinyl, pyrrolidyl, piperidyl, piperazinyl, morpholinyl, tetrahydro-2H-1,4-thiazinyl, tetrahydrofuryl, dihydrofuryl, tetrahydropyranyl, dihydropyranyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1]nonyl, azabicylo[4.3.0]nonyl, oxabicylo[2.2.1]heptyl, 1,5,9-triazacyclododecyl, and the like.

A “heteroaryl group” is intended to mean an aromatic monovalent monocyclic, bicyclic, or tricyclic radical containing 5 to 18 ring atoms, including 1 to 5 heteroatoms selected from nitrogen, oxygen and sulfur, which may be unsubstituted or substituted, and to which may be fused one or more cycloalkyl groups, heterocycloalkyl groups, or aryl groups, which themselves may be unsubstituted or substituted. Illustrative examples of heteroaryl groups include, but are not limited to, thienyl, pyrrolyl, imidazolyl, pyrazolyl, furyl, isothiazolyl, furazanyl, isoxazolyl, thiazolyl, pyridyl, pyrazinyl, pyrimidinyl, pyridazinyl, triazinyl, benzo[b]thienyl, naphtho[2,3-b]thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyridinyl, quinoxyalinyl, quinzolinyl, benzothiazolyl, benzimidazolyl, tetrahydroquinolinyl, cinnolinyl, pteridinyl, carbazolyl, beta-carbolinyl, phenanthridinyl, acridinyl, perimidinyl, phenanthrolinyl, phenazinyl, isothiazolyl, phenothiazinyl, and phenoxazinyl and the like.

The term “leaving group” as used herein refers to any group that departs from a molecule in a substitution reaction by breakage of a bond. Examples of leaving groups include, but are not limited to, halides, tosylates, arenesulfonates, alkylsulfonates, and triflates.

Suitable protecting groups are recognizable to those skilled in the art. Examples of suitable protecting groups can be found in T. Green & P. Wuts, Protective Groups in Organic Synthesis (2d ed. 1991), the disclosure of which is hereby incorporated herein by reference.

Suitable salt anions include, but are not limited to, inorganics such as halogens, pseudohalogens, sulfates, hydrogen sulfates, nitrates, hydroxides, phosphates, hydrogen phosphates, dihydrogen phosphates, perchlorates, and related complex inorganic anions, and organics such as carboxylates, sulfonates, bicarbonates and carbonates.

Examples of substituents for alkyl and aryl groups include mercapto, thioether, nitro (NO₂), amino, aryloxyl, halogen, hydroxyl, alkoxyl, and acyl, as well as aryl, cycloalkyl and saturated and partially saturated heterocycles. Examples of substituents for cycloalkyl groups include those listed above for alkyl and aryl, as well as alkyl groups.

Exemplary substituted aryls include a phenyl or naphthyl ring substituted with one or more substituents, preferably one to three substituents, independently selected from halo, hydroxy, morpholino(C₁-C₂₀)alkoxycarbonyl, pyridyl (C₁-C₂₀)alkoxycarbonyl, halo (C₁-C₂₀)alkyl, C₁-C₂₀ alkyl, C₁-C₂₀ alkoxy, carboxy, C₁-C₂₀ alkocarbonyl, carbamoyl, N—(C₁-C₂₀)alkylcarbamoyl, amino, C₁-C₂₀alkylamino, di(C₁-C₂₀)alkylamino or a group of the formula —(CH₂)ₐ—R_{b} where a can be 1, 2, 3, 4, or 5 and where R_{b} can be hydroxy, C₁-C₂₀ alkoxy, carboxy, C₁-C₂₀ alkoxycarbonyl, amino, carbamoyl, C₁-C₂₀ alkylamino or di(C₁-C₂₀)alkylamino, sulfonic acids, sulfonic esters, sulfonic amides, amides, esters and the like.

Another substituted alkyl is halo(C₁-C₂₀)alkyl, which represents a straight or branched alkyl chain having at least one halogen atom attached to it. Exemplary halo(C₁-C₂₀)alkyl groups include chloromethyl, 2-bromoethyl, 1-chloroisopropyl, 3-fluoropropyl, 2,3-dibromobutyl, 3-chloroisobutyl, trifluoromethyl, trifluoroethyl, and the like.

Another substituted alkyl is hydroxy (C₁-C₂₀)alkyl, which represents a straight or branched alkyl chain having from one to twenty carbon atoms with a hydroxy group attached to it. Exemplary hydroxy(C₁-C₂₀)alkyl groups include hydroxymethyl, 2-hydroxyethyl, 3-hydroxypropyl, 2-hydroxyisopropyl, 4-hydroxybutyl, and the like.

Yet another substituted alkyl is C₁-C₂₀ alkylthio(C₁-C₂₀)alkyl, which is a straight or branched C₁-C₂₀ alkyl group with a C₁-C₂₀ alkylthio group attached to it. Exemplary C₁-C₂₀ alkylthio(C₁-C₂₀)alkyl groups include methylthiomethyl, ethylthiomethyl, propylthiopropyl, sec-butylthiomethyl, and the like.

Yet another exemplary substituted alkyl is heterocycle(C₁-C₂₀)alkyl, which is a straight or branched alkyl chain having from one to twenty carbon atoms with a heterocycle attached to it. Exemplary heterocycle(C₁-C₂₀)alkyls include pyrrolylmethyl, quinolinylmethyl, 1-indolylethyl, 2-furylethyl, 3-thien-2-ylpropyl, 1-imidazolylisopropyl, 4-thiazolylbutyl and the like.

Yet another substituted alkyl is aryl(C₁-C₂₀)alkyl, which is a straight or branched alkyl chain having from one to twenty carbon atoms with an aryl group attached to it. Exemplary aryl(C₁-C₂₀)alkyl groups include phenylmethyl, 2-phenylethyl, 3-naphthyl-propyl, 1-naphthylisopropyl, 4-phenylbutyl and the like.

The heterocycloalkyls and the heteroaryls can, for example, be substituted with 1,2 or 3 substituents independently selected from halo, halo(C₁-C₂₀)alkyl, C₁-C₂₀ alkyl, C₁-C₂₀ alkoxy, carboxy, C₁-C₂₀ alkoxycarbonyl, carbamoyl, —(C₁-C₂₀)alkylcarbamoyl, amino, C₁-C₂₀alkylamino, di(C₁-C₂₀)alkylamino or a group having the structure —(CH₂)ₐ—R₇ where a can be 1, 2, 3, 4, or 5 and R₇ can be hydroxy, C₁-C₂₀ alkoxy, carboxy, C₁-C₂₀ alkoxycarbonyl, amino, carbamoyl, C₁-C₂₀alkylamino or di(C₁-C₂₀)alkylamino.

Examples of substituted heterocycloalkyls include, but are not limited to, 3-N-t-butyl carboxamide decahydroisoquinolinyl and 6-N-t-butyl carboxamide octahydro-thieno[3,2-c]pyridinyl. Examples of substituted heteroaryls include, but are not limited to, 3-methylimidazolyl, 3-methoxypyridyl, 4-chloroquinolinyl, 4-aminothiazolyl, 8-methylquinolinyl, 6-chloroquinoxalinyl, 3-ethylpyridyl, 6-methoxybenzimidazolyl, 4-hydroxyfuryl, 4-methylisoquinolinyl, 6,8-dibromoquinolinyl, 4,8-dimethyinaphthyl, 2-methyl-1,2,3,4-tetrahydroisoquinolinyl, N-methyl-quinolin-2-yl, 2-t-butoxycarbonyl-1,2,3,4-isoquinolin-7-yl and the like.

A “pharmaceutically acceptable solvate” is intended to mean a solvate that retains the biological effectiveness and properties of the biologically active components of the inventive compounds. Examples of pharmaceutically acceptable solvates include, but are not limited to, compounds prepared using water, isopropanol, ethanol, DMSO, and other excipients generally referred to as GRAS or likewise recognized by the United States Food and Drug Administration (FDA) as acceptable ingredients.

In the case of solid formulations, it is understood that the compounds of the invention may exist in different polymorph forms, such as stable and metastable crystalline forms (and solvates thereof) and isotropic and amorphous forms, all of which are intended to be within the scope of the present invention.

A “pharmaceutically acceptable salt” is intended to mean those salts that retain the biological effectiveness and properties of the free acids and bases and that are not biologically or otherwise undesirable. Examples of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, citrates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, hydroxybutyrates, glycolates, tartrates, methanesulfoantes, propanesulfonates, naphthalene-1-sulfonates, naphthalene-2-sulfonates, and mandelates.

If a compound of the present invention is a base, the desired salt may be prepared by any suitable method known to the art, including treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, lactic acid, salicylic acid, pyranosidyl acids such as glucuronic acid and galacturonic acid, alpha-hydroxy acids such as citric acid and tartaric acid, amino acids such as aspartic acid and glutamic acid, aromatic acids such as benzoic acid and cinnamic acid, sulfonic acids such as p-toluenesulfonic acid or ethanesulfonic acid, or the like.

If a compound of the present invention is an acid, the desired salt may be prepared by any suitable method known to the art, including treatment of the free acid with an inorganic or organic base, such as an amine (primary, secondary or tertiary), or an alkali metal or alkaline earth metal hydroxide or the like. Illustrative examples of suitable salts include organic salts derived from amino acids such as glycine and arginine; ammonia; primary, secondary and tertiary amines; cyclic amines such as piperidine, morpholine and piperazine; and inorganic salts derived from sodium, calcium, potassium, magnesium, manganese, iron, copper, zinc, aluminum, and lithium.

### EXAMPLES

Preparation of compounds according to the invention is illustrated by reference to the following non-limiting examples. It will be appreciated by persons skilled in the art with the teachings of the examples and the rest of the specification (i) how the chemistry may be applied to other peripheral groups on tetrapyrrolic ring structures that fall within the scope of this invention and (ii) that other synthetic routes may be suitable for preparation of the desired compounds.

### 2-Vinyl chlorin e6 amides

### Example 1

### 3-[[(3-hydroxypropyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (30)

Methyl pheophorbide (0.5 g) was dissolved in dichloromethane (10 mL) and aminopropan-3-ol (0.5 g) was added. The reaction was stirred overnight at room temperature and the solvent reduced to a volume of ˜2 mL by rotary evaporation. The solution was chromatographed on silica using 2% methanol/dichloromethane as eluent. The major green fraction was collected and crystallized from hexane/dichloromethane. Yield of title compound=0.6 g.

### Example 2

### 3-[[(3-hydroxypropyl)amino]carbonyl]-13-ethenyl-18-ethyl-7,8-dihydro-5-(acetic acid)-2,8,12,17-tetramethyl-21H, 23H-Porphine-7-propionic acid, (31)

To compound (30) (100 mg) dissolved in THF (50 mL) was added a solution of sodium hydroxide (200 mg) in water (1 mL). The solution was stirred at room temperature overnight and the THF removed by rotary evaporation. The residue was dissolved in water (4 mL) and acetic acid was added dropwise until the solution was neutral. Chloroform (200 mL) was added and the aqueous layer exhaustively extracted with chloroform. The organic layer was collected and dried over sodium sulfate and filtered. The solvent was reduced to ˜4 mL and the solution chromatographed on silica using 7-10% methanol dichloromethane as eluent. The major green band was collected and dried. Yield of title compound=70 mg.

### Example 3

### 3-[[(3-hydroxypropyl)amino]carbonyl]-13-ethenyl-18-ethyl-7,8-dihydro-5-(acetic acid)-2,8,12,17-tetramethyl-21H, 23H-Porphine-7-propionic acid, disodium salt (32)

To compound (31) (100 mg) was added a solution of sodium hydroxide (200 mg) in water (1 mL). The solution was stirred at room temperature for 1 hr and then loaded onto an ion exchange column (Amberlite, CG-50 ion exchange resin, weakly acidic, sodium form). The green band was collected and lyophilized. Yield of title compound=110 mg.

### Example 4

### 3-[[(3-hydroxypropyl)amino]carbonyl]-13-ethenyl-18-ethyl-7,8-dihydro-5-(1-hydroxy-2-methoxy-2-oxoethyl)-2,8,12,17-tetramethyl-21H, 23H-Porphine-7-propionic acid, methyl ester (33)

10-Hydroxy methyl pheophorbide (0.5 g) was dissolved in dichloromethane (5 mL) and aminopropan-3-ol (0.3 g) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and crystallized from methanol/dichloromethane. Yield of title compound=0.5 g.

### Example 5

### 3-[[(3-acetoxypropyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (34)

Compound (30) (100 mg) was dissolved in dichloromethane (50 mL) and pyridine (0.5 mL) and acetic anhydride (0.1 g) was added. The reaction was stirred overnight at room temperature and then poured into water. The organic layer was washed well with water and dilute sodium bicarbonate, separated and the aqueous layer back extracted with dichloromethane (50 mL). The organic layer was rotoevaporated to dryness and the crude residue dissolved in dichloromethane (5 mL) and chromatographed on silica using 1% methanol/dichloromethane as eluent. The major green fraction was collected and crystallized from methanol/dichloromethane. Yield of title compound=0.1 g.

### Example 6

### 3-[[(3-hydroxypropyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl amide (35)

Pheophorbide (300 mg) was dissolved in dichloromethane (50 mL) and tetrahydrofuran (50 mL) and triethylamine added (0.3 mL). The solution was cooled to 0° C. in an ice bath. Ethyl chloroformate (0.3 mL) was added and the solution stirred for 1 hr at room temperature. Methyl amine in THF (2.0M solution, 2.5 ml) was added and the reaction closely monitored by TLC (5% acetone/dichloromethane). When deemed complete the reaction was poured into water (100 mL) and the organic phase separated and rotoevaporated. The residue was chromatographed on silica using 2% methanol dichloromethane as eluent and the major gray fraction collected. The organic layer was removed by rotoevaporation and the product precipitated from dichloromethane/methanol. Yield of title compound=285 mg. The product was dissolved in a solution of dichloromethane (5 mL) and 3-hydroxypropylamine (300 mg) was added. The solution was stirred at room temperature for 2 days and the solution evaporated to almost dryness. The crude residue was dissolved in dichloromethane (5 mL) and chromatographed on silica using 5% methanol/dichloromethane as eluent. The major green fraction was collected and crystallized from methanol/dichloromethane. Yield of title compound=230 mg.

### Example 7

### 3-[[(3-hydroxypropyl)amino]carbonyl]-13-ethenyl-18-ethyl-7,8-dihydro-5-(2-hydroxyethyl)-2,8,12,17-tetramethyl-21H, 23H-Porphine-7-propionic acid, methyl amide (36)

Sodium borohydride (1.0 g) and lithium chloride (1.12 g) were added to a solution of dry THF (20 ml). Compound (35) (270 mg) dissolved in tetrahydrofuran (20 mL, dry) was added to the solution. After stirring at room temperature for 2 days the reaction was quenched with water (50 mL) and acetic acid (2 mL). The solution was extracted with dichloromethane (2×50 mL) and the organic layer separated, dried and evaporated to dryness. The crude residue was chromatographed on silica using 7% methanol/dichloromethane as eluent. The major green fraction was collected and precipitated from dichloromethane/hexane/ethylacetate. Yield of the title compound=170 mg.

### Example 8

### 7-(3-hydroxypropyl)-3-[[(3-hydroxypropyl)amino]carbonyl]-13-ethenyl-18-ethyl-7,8-dihydro-5-(2-hydroxyethyl)-2,8,12,17-tetramethyl-21H, 23H-Porphine (37)

Compound (30) (270 mg) was reduced with lithium borohydride and isolated as described in Example 7. Yield of the title compound=167 mg.

### Example 9

### 3-[[(2,3-dihydroxypropyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (38)

Methyl pheophorbide (0.5 g) was dissolved in tetrahydrofuran (3 mL), pyridine (2 mL) and aminopropan-2,3-diol (0.5 g) was added. The reaction was stirred overnight at room temperature and the solvent removed by rotary evaporation at low temperature. The crude residue was dissolved in dichloromethane (2 mL) and chromatographed on silica using 5% methanol/dichloromethane as eluent. The major green fraction was collected and crystallized from methanol/dichloromethane. Yield of title compound=0.5 g.

### Example 10

### 3-[[(5-hydroxy-3-oxa-pentyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (39)

Methyl pheophorbide (0.5 g) was dissolved in dichloromethane (5 mL) and 2-(2-aminoethoxy)ethanol (0.5 g) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and crystallized from a small amount of methanol/dichloromethane. Yield of title compound=0.5 g.

### Example 11

### 3-[[(5-hydroxy-3-oxa-pentyl)amino]carbonyl]-13-ethenyl-18-ethyl-7,8-dihydro-5-(acetic acid)-2,8,12,17-tetramethyl-21H, 23H-Porphine-7-propionic acid, (40)

To compound (39) (100 mg) dissolved in THF (50 mL) was added a solution of sodium hydroxide (200 mg) in water (1 mL). The solution was stirred at room temperature overnight and the THF removed by rotary evaporation. Water (4 mL) was added and acetic acid was added dropwise until a thick precipitate occurred. The precipitate was collected and dried and chromatographed on silica using 7-10% methanol dichloromethane as eluent. The major green band was collected and dried. Yield of title compound=70 mg.

### Example 12

### 3-[[(5-hydroxy-3-oxa-pentyl)amino]carbonyl]-13-ethenyl-18-ethyl-7,8-dihydro-5-(acetic acid)-2,8,12,17-tetramethyl-21H, 23H-Porphine-7-propionic acid, disodium salt (41)

To compound (11) (100 mg) was added a solution of sodium hydroxide (200 mg) in water (1 mL). The solution was stirred at room temperature for 1 hr and then loaded onto an ion exchange column (Amberlite, CG-50 ion exchange resin, weakly acidic sodium form). The green band was collected and lyophilized. Yield of title compound=110 mg.

### Example 13

### 3-[[(5-hydroxy-3-oxa-pentyl)amino]carbonyl]-13-ethenyl-18-ethyl-7,8-dihydro-5-(1-hydroxy-2-methoxy-2-oxoethyl)-2,8,12,17-tetramethyl-21H, 23H-Porphine 7-propionic acid methyl ester (42)

10-Hydroxy methyl pheophorbide (0.5 g) was dissolved in dichloromethane (5 mL) and 2-(2-aminoethoxy)ethanol (0.5 g) was added. The reaction was stirred overnight at room temperature and the solvent removed by rotary evaporation. The crude residue was dissolved in dichloromethane (2 mL) and chromatographed on silica using 4% methanol/dichloromethane as eluent. The major green fraction was collected but could not be induced to crystallize. Yield of title compound=0.5 g.

### Example 14

### 7-(3-hydroxypropyl)-3-[[(5-hydroxy-3-oxa-pentyl)amino]carbonyl]-13-ethenyl-18-ethyl-7,8-dihydro-5-(2-hydroxyethyl)-2,8,12,17-tetramethyl-21H, 23H-Porphine (43)

Compound (39) (280 mg) was reduced with lithium borohydride and isolated as described in Example 7. Yield of the title compound=172 mg.

### Example 15

### 3-[[(3,6-dioxa-heptyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (44)

Methyl pheophorbide (0.5 g) was dissolved in dichloromethane (5 mL) and 2-(2-methoxyethoxy)ethylamine (0.5 g) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and crystallized from a small amount of methanol/dichloromethane. Yield of title compound=0.5 g.

### Example 16

### 3-[[(5-acetoxy-3-oxa-pentyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (45)

Compound (39) (100 mg) was dissolved in dichloromethane (50 mL) and pyridine (0.5 mL) and acetic anhydride (0.1 g) was added. The reaction was stirred overnight at room temperature and then poured into water. The organic layer was washed well with water, saturated sodium bicarbonate solution, separated and the aqueous layer back extracted with dichloromethane (50 mL). The organic layer was rotoevaporated to dryness and the crude residue dissolved in dichloromethane (5 mL) and chromatographed on silica using 1% methanol/dichloromethane as eluent. The major green fraction was collected and crystallized from methanol/dichloromethane. Yield of title compound=0.1 g.

### Example 17

### 3-[[(3-oxa-5-aminopentyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (46)

Methyl pheophorbide (0.5 g) was dissolved in dichloromethane (5 mL) and 2-(2-aminoethoxy)ethylamine (0.5 g) was added. The reaction was stirred overnight at room temperature and methylene chloride (15 mL) was added. The crude residue was washed with water (2×100 mL) and the organic layer collected and reduced to ˜5 mL by rotary evaporation. The solution was chromatographed on silica using 5% methanoltdichloromethane/triethylamine (0.1%) as eluent. The major green fraction was collected and crystallized from a small amount of methanol/dichloromethane. Yield of title compound=0.45 g.

### Example 18

### 3-[[(5-N,N-dimethylamino-3-oxapentyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (47)

Methyl pheophorbide (0.5 g) was dissolved in dichloromethane (7 mL) and 2-[(2-aminoethoxy)ethyl]dimethylamine (0.5 g) was added. The reaction was stirred overnight at room temperature and the solvent removed by rotary evaporation. The crude residue was dissolved in dichloromethane (2 mL) and chromatographed on silica using 5% methanol/dichloromethane as eluent. The major green fraction was collected and crystallized from a small amount of methanol/dichloromethane. Yield of title compound=0.52 g.

### Example 19

### 3-[[(5-N,N,N-trimethylammonium-3-oxapentyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester iodide(48)

Compound 47 (0.1 g) was dissolved in acetone (20 mL) and iodomethane (0.1 g) was added. The reaction was stirred overnight at room temperature and the solvent removed by rotary evaporation. The crude residue was dissolved in dichloromethane (2 mL) and precipitated using hexane. The precipitate was filtered and dried. Yield of title compound=0.1 g.

### Example 20

### 3-[[(6-hydroxy-4-(2′-hydroxyethyl)-4-azahexyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (49)

Methyl pheophorbide (0.5 g) was dissolved in dichloromethane (7 mL) and N-(3-aminopropyl)diethanolamine (0.5 g) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and crystallized from a small amount of hexane/dichloromethane. Yield of title compound=0.52 g.

### Example 21

### 3-[[(6-methoxy-4-(2′-methoxyethyl)-4-azahexyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (50)

Methyl pheophorbide (0.5 g) was dissolved in dichloromethane (50 mL) and (3-aminopropyl)bis(2-methoxyethyl)amine (0.5 g) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and crystallized from a small amount of hexane/dichloromethane. Yield of title compound=0.52 g.

### Example 22

### 3-[[(6-acetoxy-4-(2′-acetoxyethyl)-4-azahexyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (51)

Compound (49) (100 mg) was dissolved in dichloromethane (50 mL) and pyridine (0.5 mL) and acetic anhydride (0.2 g) was added. The reaction was stirred overnight at room temperature and then poured into 5% sodium bicarbonate solution. The organic layer was washed well with 5% sodium bicarbonate solution, separated, and the aqueous layer back extracted with dichloromethane (50 mL). The organic layer was rotoevaporated to dryness and the crude residue dissolved in dichloromethane (5 mL) and chromatographed on silica using 7% methanol/dichloromethane as eluent. The major green fraction was collected and crystallized from methanol/dichloromethane. Yield of title compound=0.12 g.

### Example 23

### 3-[[(8-hydroxy-3,6-dioxooctyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (52)

Methyl pheophorbide (0.5 g) was dissolved in dichloromethane (7 mL) and 2-[2-[2-aminoethoxy]ethoxy]ethanol (0.5 g) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected but could not be induced to crystallize. Yield of title compound=0.54 g.

### Example 24

### 3-[[(8-methoxy-3,6-dioxooctyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid methyl ester (53)

Methyl pheophorbide (0.5 g) was dissolved in dichloromethane (50 mL) and 2-[2-(2-methoxyethoxy)ethoxy]ethylamine (0.5 g) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected but could not be induced to crystallize. Yield of title compound=0.6 g.

### Example 25

### 3-[[(3-propylphosphate)amino]carbonyl]-13-ethenyl-18-ethyl-7,8-dihydro-5-(2-methoxy-2-oxoethyl)-2,8,12,17-tetramethyl-21H, 23H-Porphine-7-propionic acid methyl ester (54)

Compound (30) (0.3 g, 0.42 mmol) was dissolved in dichloromethane (50 mL) and triethylamine (5 mL) added. The solution was cooled to 0° C. and phosphorus oxychloride (0.1 mL) added. The solution was stirred at room temperature for 2 hrs and water (50 mL) was cautiously added. The solution was extracted using chloroform and the crude residue evaporated to dryness. The residue was dissolved in dichloromethane/methanol (10%) and chromatographed on silica using 10-15% methanol/dichloromethane as eluent. The major green fraction was collected and precipitated from dichloromethane/hexane. Yield of title compound=0.16 g.

### Example 26

### 3-[[(3-propylsulfate)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (55)

Compound (30) (0.3 g) was dissolved in pyridine (5 mL) and triethylamine (5 mL) added. The solution was cooled to 0° C. and chlorosulfonic acid (0.06 mL) added. The solution was stirred at room temperature for 2 hrs and water (50 mL) was cautiously added. The solution was extracted using chloroform and the crude residue evaporated to dryness. The residue was dissolved in dichloromethane/methanol (10%) and chromatographed on silica using 10-15% methanol/dichloromethane as eluent. The major green fraction was collected and precipitated from dichloromethane/hexane. Yield of title compound=0.22 g.

### Example 27

### 2-Desvinyl-2-formyl-pheophorbide, methyl ester (9), X═H

Methyl pheophorbide (2.0 g) was dissolved in dioxane (400 mL) and the solution was purged with argon for 30 min. OsO₄ (70 mg) was added to the solution. A solution of NaIO₄ (4.75 g) in water (30 mL) and acetic acid (2 mL) was added dropwise over 40 min. The solution was stirred at room temperature overnight. Water (1 L) was added and the water was extracted with dichloromethane (2×750 mL). The organic extracts were combined and washed with aqueous Na₂SO₃ (20 g/200 mL) and water (1 L). The organic layer was separated, dried over sodium sulfate, filtered and evaporated. The residue was dissolved in dichloromethane and methanol added. The dichloromethane was removed by rotoevaporation and the precipitated producted filtered and dried under vacuum. Yield of the title compound=1.20 g

### Example 28

### 2-desvinyl-2-formyl-10-hydroxy-pheophorbide, methyl ester (9) X═OH

Using an identical procedure as in Example 27, 10-hydroxy-pheophorbide methyl ester (2.0 g) was converted to the title compound. Yield of title compound=1.12 g.

### 2-Formyl chlorin e6 amides

### Example 29

### 3-[[methylamino]carbonyl]-13-formyl-18-ethyl-7,8-dihydro-5-(2-methoxy-2-oxoethyl)-2,8,12,17-tetramethyl-21H, 23H-Porphine-7-propionic acid, methyl ester (56)

2-Desvinyl-2-formyl methyl pheophorbide (0.5 g) was dissolved in a solution of methylamine (2.0M/THF) (20 mL). The solution stirred was for 2 days at room temperature and the solvent removed by rotary evaporation. The crude residue was dissolved in dichloromethane (2 mL) and chromatographed on silica using 2% methanol/dichloromethane as eluent. The major brown/green fraction was collected and crystallized from dichloromethane/hexane. Yield of title compound=0.6 g.

### Example 30

### 3-[[pentylamino]carbonyl]-13-formyl-18-ethyl-7,8-dihydro-5-(2-methoxy-2-oxoethyl)-2,8,12,17-tetramethyl-21H, 23H-Porphine-7-propionic acid, methyl ester (57)

2-Desvinyl-2-formyl methyl pheophorbide (0.5 g) was dissolved in dichloromethane (7 mL) and pentylamine (0.5 mL) was added. The solution was stirred for 2 days at room temperature and the solvent removed by rotary evaporation. The crude residue was dissolved in dichloromethane (2 mL) and chromatographed on silica using 2% methanol/dichloromethane as eluent. The major brown/green fraction was collected and crystallized from dichloromethane/hexane. Yield of title compound=0.65 g.

### Example 31

### 3-[[3-hydroxyethyl]amino]carbonyl]-13-formyl-18-ethyl-7,8-dihydro-5-(2-methoxy-2-oxoethyl)-2,8,12,17-tetramethyl-21H, 23H-Porphine-7-propionic acid, methyl ester (58)

2-Desvinyl-2-formyl methyl pheophorbide (0.5 g) was dissolved in dichloromethane (7 mL) and 2-hydroxyethylamine (0.5 mL) was added. The solution was stirred for 2 days at room temperature and the solvent removed by rotary evaporation. The crude residue was dissolved in dichloromethane (2 mL) and chromatographed on silica using 3% methanol/dichloromethane as eluent. The major brown/green fraction was collected and crystallized from dichloromethane/hexane. Yield of title compound=0.65 g.

### Example 32

### 3-[[3-hydroxypropyl]amino]carbonyl]-13-formyl-18-ethyl-7,8-dihydro-5-(2-methoxy-2-oxoethyl)-2,8,12,17-tetramethyl-21H, 23H-Porphine-7-propionic acid, methyl ester (59)

2-Desvinyl-2-formyl methyl pheophorbide (0.5 g) was dissolved in dichloromethane (7 mL) and 3-aminopropanol (0.5 mL) was added. The solution was stirred for 2 days at room temperature and the solvent removed by rotary evaporation. The crude residue was dissolved in dichloromethane (2 mL) and chromatographed on silica using 3% methanol/dichloromethane as eluent. The major brown/green fraction was collected and crystallized from dichloromethane/hexane. Yield of title compound=0.65 g.

### Example 33

### 3-[[3-hydroxypropyl]amino]carbonyl]-13-formyl-18-ethyl-7,8-dihydro-5-(1-hydroxy-2-methoxy-2-oxoethyl)-2,8,12,17-tetramethyl-21H, 23H-Porphine-7-propionic acid, methyl ester (60)

To 2-desvinyl-2-formyl-10-hydroxypheophorbide methyl ester (120 mg) dissolved in dichloromethane (50 mL) was added 3-hydroxypropylamine (300 mg) and the solution was stirred overnight. The solvent was removed by rotoevaporation The crude residue was dissolved in dichloromethane (2 mL) and chromatographed on silica using 2% methanol/dichloromethane as eluent. The major brown/green fraction was collected and crystallized from methanol/dichloromethane. Yield of title compound=145 mg.

### Example 34

### 3-[[3-hydroxypropyl]amino]carbonyl]-13-formyl-18-ethyl-7,8-dihydro-5-(2-methoxy-2-oxoethyl)-2,8,12,17-tetramethyl-21H, 23H-Porphine-7-propionic acid, methyl amide (61)

The propionic methyl amide pheophorbide (200 mg) (prepared as described in Example 6) was dissolved in dioxane (40 mL) and the solution was purged with argon for 30 min. OsO₄ (7.0 mg) was added to the solution. A solution of NaIO₀₄ (0.5 g) in water (3.0 mL) and acetic acid (0.2 mL) was added dropwise over 40 min. The solution was stirred at room temperature overnight. Water (100 mL) was added and the water was extracted with dichloromethane (2×75 mL). The organic extracts were combined and washed with aqueous Na₂SO₃ (2.0 g/20 mL) and water (1 L). The organic layer was separated, dried over sodium sulfate, filtered and evaporated. The residue was dissolved in dichloromethane and methanol added. The dichloromethane was removed by rotoevaporation and the precipitated product filtered and dried under vacuum. Yield of 2-Desvinyl-2-formyl pheophorbide propionic methylamide=120 mg.

2-Desvinyl-2-formyl pheophobide propionic methylamide (120 mg) was dissolved in a solution of dichloromethane (3 mL) and 3-hydroxypropylamine (300 mg) was added. The solution was stirred at room temperature for 2 days and the solution evaporated to near dryness. The crude residue was dissolved in dichloromethane (5 mL) and chromatographed on silica using 1% methanol/dichloromethane as eluent. The major green fraction was collected and crystallized from methanol/dichloromethane. Yield of title compound=120 mg.

### Example 35

### 3-[[(2,3-dihydroxypropyl)amino]carbonyl]-13-formyl-18-ethyl-7,8-dihydro-5-(2-methoxy-2-oxoethyl)-2,8,12,17-tetramethyl-21H, 23H-Porphine-7-propionic acid, methyl ester (62)

2-Desvinyl-2-formyl methyl pheophorbide (0.5 g) was dissolved in tetrahydrofuran (5 mL) and pyridine (2 mL) and aminopropan-2,3-diol (0.5 g) was added. The reaction was stirred overnight at room temperature and the solvent removed by rotary evaporation. The crude residue was dissolved in dichloromethane (2 mL) and chromatographed on silica using 5% methanol/dichloromethane as eluent. The major green fraction was collected and crystallized from hexane/dichloromethane. Yield of title compound=0.5 g.

### Example 36

### 3-[[(5-hydroxy-3-oxa-pentyl)amino]carbonyl]-13-formyl-18-ethyl-7,8-dihydro-5-(2-methoxy-2-oxoethyl)-2,8,12,17-tetramethyl-21H, 23H-Porphine-7-propionic acid methyl ester (63)

2-Desvinyl-2-formyl methyl pheophorbide (0.5 g) was dissolved in dichloromethane (5 mL) and 2-(2-aminoethoxy)ethanol (0.5 g) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and crystallized from a small amount of hexane/dichloromethane. Yield of title compound=0.5 g.

### Example 37

### 3-[[(5-hydroxy-3-oxa-pentyl)amino]carbonyl]-13-formyl-18-ethyl-7,8-dihydro-5-(1-hydroxy-2-methoxy-2-oxoethyl)-2,8,12,17-tetramethyl-21H, 23H-Porphine-7-propionic acid, methyl ester (64)

2-Desvinyl-2-formyl-10-hydroxy methyl pheophorbide (50 mg) was dissolved in dichloromethane (5 mL) and 2-(2-aminoethoxy)ethanol (50 mg) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and crystallized from methanol/dichloromethane. Yield of title compound=65 mg.

### Example 38

### 3-[[(3,6-dioxa-heptyl)amino]carbonyl]-13-formyl-18-ethyl-7,8-dihydro-5-(2-methoxy-2-oxoethyl)-2,8,12,17-tetramethyl-21H, 23H-Porphine-7-propionic acid, methyl ester (65)

2-Desvinyl-2-formyl methyl pheophorbide (0.2 g) was dissolved in dichloromethane (5.0 mL) and 2-[2-(2-methoxyethoxy)ethoxy]ethylamine (0.2 g) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and crystallized from a small amount of methanol/dichloromethane. Yield of title compound=0.30 g.

### Example 39

### 3-[[(5-acetoxy-3-oxa-pentyl)amino]carbonyl]-13-formyl-18-ethyl-7,8-dihydro-5-(2-methoxy-2-oxoethyl)-2,8,12,17-tetramethyl-21H, 23H-Porphine-7-propionic acid, methyl ester (66)

2-Desvinyl-2-formyl-methyl pheophorbide (50 mg) was dissolved in dichloromethane (5 mL) and 1-acetoxy-2-(2-aminoethoxy)ethane (50 mg) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and crystallized from methanol/dichloromethane. Yield of title compound=65 mg.

### Example 40

### 3-[[(5-amino-3-oxa-pentyl)amino]carbonyl]-13-formyl-18-ethyl-7,8-dihydro-5-(2-methoxy-2-oxoethyl)-2,8,12,17-tetramethyl-21H, 23H-Porphine-7-propionic acid, methyl ester (67)

2-Desvinyl-2-formyl methyl pheophorbide (50 mg) was dissolved in dichloromethane (5 mL) and 2-(2-aminoethoxy)ethylamine (150 mg) was added. The reaction was stirred overnight at room temperature and the solvent removed by rotary evaporation. The crude residue was dissolved in dichloromethane (2 mL) and chromatographed on silica using 2% methanol/dichloromethane as eluent. The major brown/green fraction was collected and crystallized from a small amount of methanol/dichloromethane. Yield of title compound=45 mg.

### Example 41

### 3-[[(5-N,N-dimethylamino-3-oxa-pentyl)amino]carbonyl-13-formyl-18-ethyl-7,8-dihydro-5-(2-methoxy-2-oxoethyl)-2,8,12,17-tetramethyl-21H, 23H-Porphine-7-propionic acid methyl ester (68)

2-Desvinyl-2-formyl methyl pheophorbide (50 mg) was dissolved in dichloromethane (5 mL) and [2-(2aminoethoxy)ethyl]dimethylamine (150 mg) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and crystallized from a small amount of methanol/dichloromethane. Yield of title compound=62 mg.

### Example 42

### 3-[[(5-N,N,N-trimethylammonium-3-oxa-pentyl)amino]carbonyl]-13-formyl-18-ethyl-7,8-dihydro-5-(2-methoxy-2-oxoethyl)-2,8,12,17-tetramethyl-21H, 23H-Porphine-7-propionic acid methyl ester iodide (69)

Compound 68 (45 mg) was dissolved in acetone (5 mL) and iodomethane (0.2 g) was added. The reaction was stirred overnight at room temperature and the solvent removed by rotary evaporation. The crude residue was dissolved in dichloromethane (2 mL) and precipitated using hexane. The precipitate was filtered and dried. Yield of title compound=50 mg.

### Example 43

### 3-[[(6-hydroxy-4-(2-hydroxyethyl)-4-azahexyl)amino]carbonyl]-13-formyl-18-ethyl-7,8-dihydro-5-(2-methoxy-2-oxoethyl)-2,8,12,17-tetramethyl-21H, 23H-Porphine-7-propionic acid methyl ester (70)

2-Desvinyl-2-formyl methyl pheophorbide (50 mg) was dissolved in dichloromethane (5 mL) and N-(3-aminopropyl)diethanolamine (150 mg) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and crystallized from a small amount of hexane/dichloromethane. Yield of title compound=56 mg.

### Example 44

### 3-[[(6-methoxy-4-(2-methoxyethyl)-4-azahexyl)amino]carbonyl]-13-formyl-18-ethyl-7,8-dihydro-5-(2-methoxy-2-oxoethyl)-2,8,12,17-tetramethyl-21H, 23H-Porphine-7-propionic acid, methyl ester (71)

2-Desvinyl-2-formyl methyl pheophorbide (50 mg) was dissolved in dichloromethane (10 mL) and 3-(N,N-(dimethoxyethyl)amino)propylamine (50 mg) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and crystallized from a small amount of hexane/dichloromethane. Yield of title compound=60 mg.

### Example 45

### 3-[[(3-N,N-bisacetoxyethyl)aminopropyl]aminocarbonyl]-13-formyl-18-ethyl-7,8-dihydro-5-(2-methoxy-2-oxoethyl)-2,8,12,17-tetramethyl-21H, 23H-Porphine-7-propionic acid, methyl ester (72)

2-Desvinyl-2-formyl methyl pheophorbide (50 mg) was dissolved in dichloromethane (5 mL) and 3-(N,N-(diacetoxyethyl)amino)propylamine (50 mg) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and crystallized from a small amount of hexane/dichloromethane. Yield of title compound=57 mg.

### Example 46

### 3-[[(8-hydroxy-3,6-dioxooctyl)amino]carbonyl]-13-formyl-18-ethyl-7,8-dihydro-5-(2-methoxy-2-oxoethyl)-2,8,12,17-tetramethyl-21H, 23H-Porphine-7-propionic acid, methyl ester (73)

2-Desvinyl-2-formyl methyl pheophorbide (50 mg) was dissolved in dichloromethane (5 mL) and 2-[2-[2-aminoethoxy]ethoxy]ethanol (150 mg) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected but could not be induced to crystallize. Yield of title compound=64 mg.

### Example 47

### 3-[[(8-methoxy-3,6-dioxooctyl)amino]carbonyl]-13-formyl-18-ethyl-7,8-dihydro-5-(2-methoxy-2-oxoethyl)-2,8,12,17-tetramethyl-21H, 23H-Porphine-7-propionic acid, methyl ester (74)

2-Desvinyl-2-formyl methyl pheophorbide (50 mg) was dissolved in dichloromethane (5 mL) and 2-[2-(2-methoxyethoxy)ethoxy]ethylamine (150 mg) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected but could not be induced to crystallize. Yield of title compound=65 mg.

### 2-Desvinyl-2-hydroxymethyl chlorin e6 amides

### Example 48

### 2-Desvinyl-2-hydroxymethyl-pheophorbide methyl ester (2) X═H

2-Desvinyl-2-formyl methyl pheophorbide (0.5 g) was dissolved in dichloromethane (dry, 70 mL). To a cold (˜4° C.) solution of tetrabutylammonium borohydride (600 mg) in dichloromethane (15 mL), 0.4 mL of acetic acid was added. The pheophorbide solution was added to the cold tetrabutylammonium borohydride solution and the resulting solution stirred for 5 hrs at (˜4° C.). The reaction was quenched with 2% acetic acid/water (100 mL) and the dichloromethane layer separated. The organic layer was evaporated to dryness and the residue dissolved in dichloromethane and chromatographed on silica using 10% acetone/dichloromethane as eluent. The major fraction was collected, evaporated and crystallized from hexane/dichloromethane. Yield=0.48 g

Two methods were used to synthesize 2-Desvinyl-2-hydroxymethyl chlorin e6 amides.

### General Method C

### Pheophorbide Ring Opening Method

2-Desvinyl-2-hydroxymethyl-pheophorbide methyl ester (0.1 g) was dissolved in dichloromethane (or THF) (20 mL) and the reacting amine added (0.2 g). The solution was stirred at room temperature for 1 or 2 days (depending on the rate of the reaction) and once complete by TLC, the solvent was removed by rotary evaporation. The crude residue was chromatographed on silica using 2-10% methanol as eluent and the major fraction collected, evaporated and crystallized or precipitated from methanol/dichloromethane or hexane/dichloromethane. Yield of the 2-Desvinyl-2-hydroxymethyl chlorin e6 amides is generally between 70-90%.

### Method D

### Reduction of 2-Desvinyl-2-formyl Chlorin e6 Amides

2-Desvinyl-2-formyl chlorin e6 amide (0.2 g) is dissolved in dichloromethane or THF (dry, 50 mL). A solution of tetrabutylammonium borohydride (600 mg) was dissolved in dichloromethane (15 mL). The reducing solution was cooled to ˜4° C. in an ice bath. The pheophorbide solution was added to the borohydride solution and the resulting solution stirred for 5 hrs. The reaction was quenched with 2% acetic acid/water (100 mL) and the dichloromethane layer separated. The organic layer was evaporated to dryness and the residue dissolved in dichloromethane and chromatographed on silica using 10%-25% acetone/dichloromethane as eluent. The major fraction was collected, evaporated and crystallized from hexane/dichloromethane. Yield is generally on the order of 80-95%.

### Example 49

### 3-[[methylamino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (75)

Synthesized according to method C using methylamine in a THF solution (2M). The compound was precipitated from methanol/dichloromethane. Yield of title compound=95%.

### Example 50

### 3-[[hexylamino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (76)

Synthesized by method C in dichloromethane using hexylamine. The compound was precipitated from methanol/dichloromethane. Yield of title compound=95%.

### Example 51

### 3-[[(2-hydroxyethyl)amino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (77)

Synthesized from compound (58) by Method D using dichloromethane as solvent. The compound was precipitated from hexane/dichloromethane. Yield of title compound=80%.

### Example 52

### 3-[[(3-hydroxypropyl)amino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl amide (78)

Synthesized from compound (59) using method D using dichloromethane as solvent. The compound was precipitated from hexane/dichloromethane. Yield of title compound=83%.

### Example 53

### 3-[[(3-hydroxypropyl)amino]carbonyl]-13-hydroxymethyl-18-ethyl-7,8-dihydro-5-(2-acetic acid)-2,8,12,17-tetramethyl-21H, 23H-Porphine-7-propionic acid (79)

Synthesized from compound (78) using the method described in Example 2. Yield of title compound=83%.

### Example 54

### 3-[[(3-hydroxypropyl)amino]carbonyl]-13-hydroxymethyl-18-ethyl-7,8-dihydro-5-(2-acetic acid)-2,8,12,17-tetramethyl-21H, 23H-Porphine-7-propionic acid, disodium salt (80)

Synthesized from compound (79) using the method described in Example 3. Yield of title compound=89%.

### Example 55

### 3-[[(3-hydroxypropyl)amino]carbonyl]-13-hydroxymethyl-18-ethyl-7,8-dihydro-5-(1-hydroxy-2-methoxy-2-oxoethyl)-2,8,12,17-tetramethyl-21H, 23H-Porphine-7-propionic acid, methyl ester (81)

Synthesized from compound (60) by Method D using dichloromethane as solvent. The compound was precipitated from hexane/dichloromethane. Yield of title compound=72%.

### Example 56

### 3-[[(5-hydroxypropyl)amino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (82)

Synthesized from compound (61) by method D using dichloromethane as solvent. The compound was precipitated from hexane/dichloromethane. Yield of title compound=85%

### Example 57

### 3-[[(2,3-dihydroxypropyl)amino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (83)

Synthesized from compound (62) by method D using dichloromethane as solvent. The compound was precipitated from hexane/dichloromethane. Yield of title compound=85%

### Example 58

### 3-[[(5-hydroxy-3-oxa-pentyl)amino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (84)

Synthesized by method C using dichloromethane and 2-(2-aminoethoxy)ethanol, and also by method D from compound (63) using dichloromethane. The compound was precipitated from hexane/dichloromethane. Yield of title compound=90% and 85%, respectively.

### Example 59

### 3-[[(5-hydroxy-3-oxa-pentyl)amino]carbonyl]-13-hydroxymethyl-18-ethyl-7,8-dihydro-5-(2-acetic acid)-2,8,12,17-tetramethyl-21H, 23H-Porphine-7-propionic acid, (85)

Synthesized from compound (84) as described in Example 2. Yield of title compound=75%.

### Example 60

### 3-[[(5-hydroxy-3-oxa-pentyl)amino]carbonyl]-13-hydroxymethyl-18-ethyl-7,8-dihydro-5-(2-acetic acid)-2,8,12,17-tetramethyl-21H, 23H-Porphine-7-propionic acid, disodium salt (86)

Synthesized from compound (85) according to the method described in Example 3. Yield=95%

### Example 61

### 3-[[(5-hydroxy-3-oxa-pentyl)amino]carbonyl]-13-hydroxymethyl-18-ethyl-7,8-dihydro-5-(1-hydroxy-2-methoxy-2-oxoethyl)-2,8,12,17-tetramethyl-21H, 23H-Porphine-7-propionic acid, methyl ester (87)

Synthesized from compound (64) by method D using dichloromethane as solvent. The compound was precipitated from hexane/dichloromethane. Yield of title compound=84%.

### Example 62

### 3-[[(3,6-dioxa-heptyl)amino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (88)

Synthesized from compound (65) by method D using dichloromethane as solvent. The compound was precipitated from hexane/dichloromethane. Yield of title compound=84%.

### Example 63

### 3-[[(5-acetoxy-3-oxa-pentyl)amino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (89)

Synthesized by method C using 3-acetoxypropylamine and dichloromethane as solvent. The compound was precipitated from hexane/dichloromethane. Yield of title compound=87%.

### Example 64

### 3-[[(5-amino-3-oxa-heptyl)amino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (90)

Synthesized from compound (67) by method D using dichloromethane as solvent. The compound was precipitated from hexane/dichloromethane. Yield of title compound=85%.

### Example 65

### 3-[[(5-N,N-dimethylamino-3-oxa-pentyl)amino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (91)

Synthesized from compound (68) by method D using dichloromethane as solvent. The compound was precipitated from hexane/dichloromethane. Yield of title compound=85%.

### Example 66

### 3-[[(5-N,N,N-trimethylamino-3-oxa-pentyl)amino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (92)

Synthesized from compound (91) using the procedure described in Example 19. Yield=89%.

### Example 67

### 3-[[(6-hydroxy-4-(2-hydroxyethyl)-4-azahexyl)amino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (93)

Synthesized from compound (70) by method D using dichloromethane as solvent. The compound was precipitated from hexane/dichloromethane. Yield of title compound=80%.

### Example 68

### 3-[[(6-methoxy-4-(2-methoxyethyl)-4-azahexyl)amino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (94)

Synthesized from compound (71) by method D using dichloromethane as solvent. The compound was precipitated from hexane/dichloromethane. Yield of title compound=87%.

### Example 69

### 3-[[(6-acetoxy-4-(2-acetoxyethyl)-4-azahexyl)amino]carbonyl]-13-hydroxymethyl-18-ethyl-7,8-dihydro-5-(2-methoxy-2-oxoethyl)-2,8,12,17-tetramethyl-21H, 23H-Porphine-7-propionic acid, methyl ester (95)

Synthesized from compound (72) by method D using dichloromethane as solvent. The compound was precipitated from hexane/dichloromethane. Yield of title compound=78%.

### Example 70

### 3-[[(8-hydroxy-3,6-dioxooctyl)amino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester (96)

Synthesized by Method C using 2-[2-[2-aminoethoxy]ethoxy]ethanol and dichloromethane as solvent. The compound was precipitated from hexane/dichloromethane. Yield of title compound=88%.

### Example 71

### 3-[[(3,6,9-trioxadecyl)amino]carbonyl]-13-acetyl-18-ethyl-7,8-dihydro-5-(2-methoxy-2-oxoethyl)-2,8,12,17-tetramethyl-21H, 23H-Porphine-7-propionic acid methyl ester (97)

Synthesized from compound (74) by method D using dichloromethane as solvent. The compound was precipitated from hexane/dichloromethane. Yield of title compound=83%.

### 2-Desvinyl-2-acetyl chlorin e6 amides

### General method E

### 2-Desvinyl-2-acetyl methyl Pheophorbide ring opening Method

2-Desvinyl-2-acetyl-pheophorbide methyl ester (0.1 g) was dissolved in dichloromethane (or THF) (5.0 mL) and the reacting amine was added (0.2 g). The solution was stirred at room temperature for 1 or 2 days (depending on the rate of the reaction) and once complete by TLC, the solvent was removed by rotary evaporation. The crude residue was chromatographed on silica using 2-10% methanol as eluent and the major fraction collected, evaporated and crystallized or precipitated from methanol/dichloromethane or hexane/dichloromethane. Yield of the 2-Desvinyl-2-acetyl chlorin e6 amides is generally between 70-90%.

### Example 72

### 3-[[methylamino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester (98)

Synthesized from method E using methylamine in THF with stirring for 2 days. The compound was precipitated from methanol/dichloromethane. Yield of title compound=0.1 g.

### Example 73

### 3-[[2-hydroxyethylamino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester (99)

Synthesized from method E using 2-aminoethanol in dichloromethane with stirring for 1 day. The compound was precipitated from hexane/dichloromethane. Yield of title compound=0.1 g.

### Example 74

### 3-[[(1-hexylamino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester (100)

Synthesized from method E using hexylamine in dichloromethane with stirring for 1 day. The compound was precipitated from hexane/dichloromethane. Yield of title compound=0.1 g.

### Example 75

### 3-[[(3-hydroxypropyl]amino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester (101)

Synthesized from method E using 5-aminopropanol in dichloromethane with stirring for 1 day. The compound was precipitated from hexane/dichloromethane. Yield of title compound=0.12 g.

### Example 76

### 3-[[(3-hydroxypropyl)amino]carbonyl]-13-acetyl-18-ethyl-7,8-dihydro-5-(2-acetic acid)-2,8,12,17-tetramethyl-21H, 23H-Porphine-7-propionic acid, (102)

To compound (101) (100 mg) dissolved in THF (50 mL) was added a solution of sodium hydroxide (200 mg) in water (1 mL). The solution was stirred at room temperature overnight and the THF removed by rotary evaporation. Water (4 mL) was added and acetic acid was added dropwise until neutral. The compound was extracted with chloroform and the organic layers combined and evaporated to ˜5 mL. The solution was chromatographed on silica using 7-10% methanol/dichloromethane as eluent. The major green band was collected and dried. Yield of title compound=70 mg.

### Example 77

### 3-[[(3-hydroxypropyl)amino]carbonyl]-13-acetyl-18-ethyl-7,8-dihydro-5-(2-acetic acid)-2,8,12,17-tetramethyl-21H, 23H-Porphine-7-propionic acid, disodium salt (103).

To compound (102) (50 mg) was added a solution of sodium hydroxide (50 mg) in water (1 mL). The solution was stirred at room temperature for 1 hr and then loaded onto an ion exchange column (Amberlite, CG-50 ion exchange resin, weakly acidic sodium form). The green band was collected and lyophilized. Yield of title compound=60 mg.

### Example 78

### 3-[[(3-hydroxypropyl)amino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl amide (104)

2-Desvinyl-2-acetyl pheophorbide (300 mg) was dissolved in dichloromethane (50 mL) and tetrahydrofuran (50 mL) and triethylamine added (0.3 mL). The solution was cooled to 0° C. in an ice bath. Ethyl chloroformate (0.3 mL) was added and the solution stirred for 1 hr at room temperature. Methyl amine in THF (2.0M solution, 2.5 ml) was added and the reaction closely monitored by TLC (5% acetone/dichloromethane). When deemed complete the reaction was poured into water (100 mL) and the organic phase separated and rotoevaporated. The residue was chromatographed on silica using 2% methanol/dichloromethane as eluent and the major gray fraction collected. The organic layer was removed by rotoevaporation and the 2-desvinyl-2-acetyl propionic methylamide pheophorbide product precipitated from dichloromethane/methanol. Yield of title compound=289 mg. The product was dissolved in a solution of dichloromethane and 3-hydroxypropylamine (300 mg) was added. The solution was stirred at room temperature for 2 days and evaporated to near dryness. The crude residue was dissolved in dichloromethane (5 mL) and chromatographed on silica using 1% methanol/dichloromethane as eluent. The major green fraction was collected and crystallized from methanol/dichloromethane. Yield of title compound=230 mg.

### Example 79

### 3-[[(2,3-dihydroxypropyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (105)

Synthesized from method E using aminopropan-2,3-diol in dichloromethane with stirring for 2 days. The compound was precipitated from hexane/dichloromethane. Yield of title compound=0.15 g.

### Example 80

### 3-[[(5-hydroxy-3-oxa-pentyl)amino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester (106)

Synthesized from method E using 2-(2-aminoethoxy)ethanol in dichloromethane with stirring for 2 days. The compound was precipitated from hexane/dichloromethane. Yield of title compound=0.17 g

### Example 81

### 3-[[(5-hydroxy-3-oxa-pentyl)amino]carbonyl]-13-acetyl-18-ethyl-7,8-dihydro-5-(2-acetic acid)-2,8,12,17-tetramethyl-21H, 23H-Porphine-7-propionic acid, (107)

To compound (106) (100 mg) dissolved in THF (50 mL) was added a solution of sodium hydroxide (200 mg) in water (1 mL). The solution was stirred at room temperature overnight and the THF removed by rotary evaporation. Water (4 mL) was added and acetic acid was added dropwise until the solution was slightly acidic. The aqueous solution was extracted with methylene chloride/pyridine (0.5%) and the solvent removed by rotary evaporation. The crude residue was chromatographed on silica using 7-13% methanol/dichloromethane as eluent. The major green/brown band was collected and dried. Yield of title compound=50 mg.

### Example 82

### 3-[[(5-hydroxy-3-oxa-pentyl)amino]carbonyl]-13-acetyl-18-ethyl-7,8-dihydro-5-(2-acetic acid)-2,8,12,17-tetramethyl-21H, 23H-Porphine-7-propionic acid, disodium salt (108).

To compound (107) (50 mg) was added a solution of sodium hydroxide (200 mg) in water (1 mL). The solution was stirred at room temperature for 1 hr and then loaded onto an ion exchange column (Amberlite, CG-50 ion exchange resin, weakly acidic sodium form). The green band was collected and lyophilized. Yield of title compound=110 mg.

### Example 83

### 3-[[(3,6-dioxa-heptyl)amino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester (109)

Synthesized from method E using 2-(2-methoxyethoxy)ethylamine in dichloromethane with stirring for 2 days. The compound was precipitated from hexane/dichloromethane. Yield of title compound=0.17 g.

### Example 84

### 3-[[(5-acetoxy-3-oxa-pentyl)amino]carbonyl]-13-ethenyl-18-ethyl-7,8-dihydro-5-(2-methoxy-2-oxoethyl)-2,8,12,17-tetramethyl-21H, 23H-Porphine-7-propionic acid, methyl ester (110)

Compound (108) (100 mg) was dissolved in dichloromethane (50 mL) and pyridine (0.5 mL) and acetic anhydride (0.1 g) was added. The reaction was stirred overnight at room temperature and then poured into water. The organic layer was washed well with water, separated and the aqueous layer back extracted with dichloromethane (50 mL). The organic layer was rotoevaporated to dryness and the crude residue was dissolved in dichloromethane (5 mL) and chromatographed on silica using 1% methanol/dichloromethane as eluent. The major green fraction was collected and crystallized from methanol/dichloromethane. Yield of title compound=0.1 g.

### Example 85

### 3-[[(5-amino-3-oxapentyl)amino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester (111)

Synthesized from method E using 2-(2-aminoethoxy)ethylamine in dichloromethane with stirring 2 days. The compound was precipitated from hexane/dichloromethane. Yield of title compound=0.17 g.

### Example 86

### 3-[[(5-N,N-dimethylamino-3-oxapentyl)amino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester (112)

Synthesized from method E using [2-(2-aminoethoxy)ethyl]dimethylamine in dichloromethane with stirring for 2 days. The compound was precipitated from hexane/dichloromethane. Yield of title compound=0.18 g.

### Example 87

### 3-[[(5-N,N,N-trimethylammonium-3-oxapentyl)amino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester iodide (113)

Compound 112 (0.1 g) was dissolved in acetone (20 mL) and iodomethane (0.1 g) was added. The reaction was stirred overnight at room temperature and the solvent removed by rotary evaporation. The crude residue was dissolved in dichloromethane (2 mL) and precipitated using hexane. The precipitate was filtered and dried. Yield of title compound=0.1 g.

### Example 88

### 3-[[(6-hydroxy-4-(2′-hydroxyethyl)-4-azahexyl)amino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester (114)

Synthesized from method E using N-(3-aminopropyl)diethanolamine in dichloromethane with stirring for 2 days. The compound was precipitated from hexane/dichloromethane. Yield of title compound=0.17 g.

### Example 89

### 3-[[(6-methoxy-4-(2′-methoxyethyl)-4-azahexyl)amino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester (115)

Synthesized from method E using N-(3-aminopropyl)dimethoxyethanolamine in dichloromethane with stirring for 2 days. The compound was precipitated from hexane/dichloromethane. Yield of title compound=0.16 g.

### Example 90

### 3-[[(6-acetoxy-4-(2′-acetoxyethyl)-4-azahexyl)amino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester (116)

Compound (114) (100 mg) was dissolved in dichloromethane (50 mL) and pyridine (0.5 mL) and acetic anhydride (0.1 g) was added. The reaction was stirred overnight at room temperature and then poured into water. The organic layer was washed well with 5% sodium bicarbonate solution, separated, and the aqueous layer back extracted with dichloromethane (50 mL). The organic layer was rotoevaporated to dryness and the crude residue was dissolved in dichloromethane (5 mL) and chromatographed on silica using 7% methanol/dichloromethane as eluent. The major green fraction was collected and crystallized from methanol/dichloromethane. Yield of title compound=0.13 g.

### Example 91

### 3-[[(8-hydroxy-3,6-dioxaoctyl)amino]carbonyl]-13-acetyl-18-ethyl-7,8-dihydro-5-(2-methoxy-2-oxoethyl)-2,8,12,17-tetramethyl-21H, 23H-Porphine-7-propionic acid, methyl ester (117)

Synthesized from method E using 2-[2-[2-aminoethoxy]ethoxy]ethanol in dichloromethane with stirring for 2 days. The compound was precipitated from hexane/dichloromethane. Yield of title compound=0.16 g

### Example 92

### 3-[[(3,6,9-trioxadecyl)amino]carbonyl]-13-acetyl-18-ethyl-7,8-dihydro-5-(2-methoxy-2-oxoethyl)-2,8,12,17-tetramethyl-21H, 23H-Porphine-7-propionic acid methyl ester (118)

Synthesized from method E using 2-(2-methoxyethoxy)ethylamine in dichloromethane with stirring for 2 days. The compound was precipitated from hexane/dichloromethane. Yield of title compound=0.16 g.

### 2-Desvinyl-2-ethyl Chlorin e6 amides

### Example 93

### Meso-pheophorbide methyl ester (3)

Methyl pheophorbide (1.0 g) was dissolved in acetone and Pd/C (0.5 g, 10%) was added. The solution was degassed twice and hydrogen introduced into the vessel via a balloon. The solution was stirred overnight at room temperature, after which TLC and UV showed the reaction to be complete. The catalyst was filtered from the solution and the solvent was removed by rotoevaporation. The residue was dissolved in dichloromethane (100 mL) and methanol (100 mL) was added. The dichloromethane was removed by rotoevaporation and the precipitated solid collected by filtration, washed with methanol (50 mL) and dried. Yield of title compound=0.85 g. The meso-chlorin e6 amide analogs were prepared using general Method F.

### Method F

Meso-pheophorbide methyl ester (0.2 g) was dissolved in dichloromethane (5.0 mL) (or THF (5.0 mL)) and the reacting amine (0.4 g) was added. The reaction was stirred overnight at room temperature and the solvent removed by rotary evaporation. The crude residue was dissolved in dichloromethane (2 mL) and chromatographed on silica using 2% methanol/dichloromethane as eluent. The major green fraction was collected and precipitated from hexane/dichloromethane. General yield of the compound=80-90%.

### Example 94

### 3-[[methylamino]carbonyl]-13,18-diethyl-7,8-dihydro-5-(2-methoxy-2-oxoethyl)-2,8,12,17-tetramethyl-21H, 23H-Porphine-7-propionic acid, methyl ester (119)

Synthesized according to method F using methylamine in a THF solution (2M). Yield of title compound=95%.

### Example 95

### 3-[[hexylamino]carbonyl]-13,18-diethyl-7,8-dihydro-5-(2-methoxy-2-oxoethyl)-2,8,12,17-tetramethyl-21H, 23H-porphine-7-propionic acid, methyl ester (120)

Synthesized by method F in dichloromethane using hexylamine. Yield of title compound=95%.

### Example 96

### 3-[[(3-hydroxypropyl)amino]carbonyl]-13,18-diethyl-7,8-dihydro-5-(2-methoxy-2-oxoethyl)-2,8,12,17-tetramethyl-21H, 23H-Porphine-7-propionic acid, methyl ester (121)

Synthesized by Method F in dichloromethane using 3-aminopropanol. Yield of title compound=80%.

### Example 97

### 3-[[(3-hydroxypropyl)amino]carbonyl]-13,18-diethyl-7,8-dihydro-5-(1-hydroxy-2-methoxy-2-oxoethyl)-2,8,12,17-tetramethyl-21H, 23H-Porphine-7-propionic acid methyl ester (122)

Meso-methylpyrropheophorbide (200 mg) was converted to meso-10-hydroxymethylpyrropheophorbide (127 mg) via the method outlined by Holt, A.S, Can. J. Biochem. Physiol. 36, 439 (1958) using quinone, in alkaline pyridine. Meso-10-hydroxymethylpyrropheophorbide (50 mg) was dissolved in dichloromethane (1 ml) and 3-aminopropanol (0.1 ml) was added. The solution was stirred overnight at room temperature and the solvent was chromatographed on silica using 5-7% methanol/dichloromethane as eluent. The major green fraction was collected and evaporated, but could not be induced to crystallize. Yield=35 mg.

### Example 98

### 3-[[(2,3-dihydroxypropyl)amino]carbonyl]-13,18-diethyl-7,8-dihydro-5-(2-methoxy-2-oxoethyl)-2,8,12,17-tetramethyl-21H,23H-Porphine-7-propionic acid, methyl ester (123)

Synthesized by method F using 2,3-dihydroxypropylamine with tetrahydrofuran as solvent. The compound was precipitated from hexane/dichloromethane. Yield of title compound=92%.

### Example 99

### 3-[[(5-hydroxy-3-oxapentyl)amino]carbonyl]-13,18-diethyl-7,8-dihydro-5-(2-methoxy-2-oxoethyl)-2,8,12,17-tetramethyl-21H,23H-Porphine-7-propionic acid, methyl ester (124)

Synthesized by method F using 2-(2-aminoethoxy)ethanol with dichloromethane as solvent. The compound was precipitated from hexane/dichloromethane. Yield of title compound=85%.

### Example 100

### 3-[[(3,6-dioxaheptyl)amino]carbonyl]-13,18-diethyl-7,8-dihydro-5-(2-methoxy-2-oxoethyl)-2,8,12,17-tetramethyl-21H,23H-Porphine-7-propionic acid, methyl ester (125)

Synthesized by method F using 2-[2-(2-methoxyethoxy)ethoxy]ethylamine with dichloromethane as solvent. The compound was precipitated from hexane/dichloromethane. Yield=85%.

### Example 101

### 3-[[(5-acetoxy-3-oxapentyl)amino]carbonyl]-13,18-diethyl-7,8-dihydro-5-(2-methoxy-2-oxoethyl)-2,8,12,17-tetramethyl-21H,23H-Porphine-7-propionic acid, methyl ester (126)

Synthesized by method F using 2-[2-(2-acetoxyethoxy)ethoxy]ethylamine with dichloromethane as solvent. The compound was precipitated from hexane/dichloromethane. Yield of title compound=86%.

### Example 102

### 3-[[(5-amino-3-oxapentyl)amino]carbonyl]-13,18-diethyl-7,8-dihydro-5-(2-methoxy-2-oxoethyl)-2,8,12,17-tetramethyl-21H,23H-Porphine-7-propionic acid, methyl ester (127)

Synthesized by Method F using 2-(2-aminoethoxy)ethylamine with dichloromethane as solvent. The compound was precipitated from hexane/dichloromethane. Yield of title compound=85%.

### Example 103

### 3-[[(5-N,N-dimethylamino-3-oxapentyl)amino]carbonyl]-13,18-diethyl-7,8-dihydro-5-(2-methoxy-2-oxoethyl)-2,8,12,17-tetramethyl-21H, 23H-Porphine-7-propionic acid, methyl ester (128)

Synthesized by Method F using [2-(2-aminoethoxy)ethyl]dimethylamine with dichloromethane as solvent. The compound was precipitated from hexane/dichloromethane. Yield of title compound=82%.

### Example 104

### 3-[[(8-N,N,N-trimethylammonium-3,6-di-oxaoctyl)amino]carbonyl]-13,18-diethyl-7,8-dihydro-5-(2-methoxy-2-oxoethyl)-2,8,12,17-tetramethyl-21H, 23H-Porphine-7-propionic acid, methyl ester iodide (129)

Synthesized from compound (128) as described in Example (114). The compound was precipitated from hexane/dichloromethane. Yield of title compound=82%.

### Example 105

### 3-[[(6-hydroxy-4-(2′-hydroxyethyl)-4-azahexyl)amino]carbonyl]-13,18-diethyl-7,8-dihydro-5-(2-methoxy-2-oxoethyl)-2,8,12,17-tetramethyl-21H, 23H-Porphine-7-propionic acid, methyl ester (130)

Synthesized by method F using N-(3-aminopropyl)diethanolamine with dichloromethane as solvent. The compound was precipitated from hexane/dichloromethane. Yield of title compound=85%.

### Example 106

### 3-[[(6-methoxy-4-(2-methoxyethyl)-4-azahexyl)amino]carbonyl]-13,18-diethyl-7,8-dihydro-5-(2-methoxy-2-oxoethyl)-2,8,12,17-tetramethyl-21H, 23H-Porphine-7-propionic acid, methyl ester (131)

Synthesized by method F using N-(3-aminopropyl)dimethoxyethanolamine with dichloromethane as solvent. The compound was precipitated from hexane/dichloromethane. Yield of title compound=91%.

### Example 107

### 3-[[(6-acetoxy-4-(2-acetoxyethyl)-4-azahexyl)amino]carbonyl]-13,18-diethyl-7,8-dihydro-5-(2-methoxy-2-oxoethyl)-2,8,12,17-tetramethyl-21H, 23H-Porphine-7-propionic acid, methyl ester (132)

Compound 130 was acetylated according to Example 111. The compound was precipitated from hexane/dichloromethane. Yield of title compound=89%.

### Example 108

### 3-[[(8-hydroxy-3,6-dioxooctyl)amino]carbonyl]-13,18-diethyl-7,8-dihydro-5-(2-methoxy-2-oxoethyl)-2,8,12,17-tetramethyl-21H,23H-porphine-7-propionic acid, methyl ester (133)

Synthesized by method F using 2-[2-[2-aminoethoxy]ethoxy]ethanol with dichloromethane as solvent. The compound was precipitated from hexane/dichloromethane. Yield of title compound=86%.

### Example 109

### 3-[[(8-methoxy-3,6-dioxooctyl)amino]carbonyl]-13,18-diethyl-7,8-dihydro-5-(2-methoxy-2-oxoethyl)-2,8,12,17-tetramethyl-21H,23H-Porphine-7-propionic acid methyl ester (134)

Synthesized by method F using 2-[2-(methoxyethoxy)ethoxy]ethylamine with dichloromethane as solvent. The compound was precipitated from hexane/dichloromethane. Yield of title compound=81%.

### 2-Desvinyl-2-(2-hydroxyethyl) Chlorin e6 amides

### Example 110

### 2-Desvinyl-2-(2-hydroxyethyl)pheophorbide methyl ester (10) (X═H, R═H)

2-Desvinyl-2-(2-hydroxyethyl)pheophorbide methyl ester was made via the method outlined by Smith K. M. et al, J. Org.Chem., 45, 2218-2224,1980, where 2-(2-hydroxyethyl)chlorin e6 trimethyl ester is converted to the pheophorbide via base catalyzed cyclization. Yield=65%

The 2-desvinyl-2-(2-hydroxyethyl) chlorin e6 amide analogs were prepared using general method G.

### General Method G

2-Desvinyl-2-(2-hydroxyethyl) pheophorbide methyl ester (0.3 g) was dissolved in dichloromethane or THF (5 mL) and the reacting amine (0.6 g) was added. The reaction was stirred overnight at room temperature and the solvent removed by rotary evaporation. The crude residue was dissolved in dichloromethane (2 mL) and chromatographed on silica using 1-5% methanol/dichloromethane as eluent. The major green fraction was collected (and rechromatographed), evaporated and precipitated from hexane/dichloromethane. General yield of the compounds=70-90%.

### Example 111

### 3-[[methylamino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid, methyl ester (135)

Synthesized according to method G using methylamine in a THF solution (2M). Yield of title compound=80%.

### Example 112

### 3-[[hexylamino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid, methyl ester (136)

Synthesized by method G in dichloromethane using hexylamine. Yield of title compound=80%.

### Example 113

### 3-[[(3-hydroxypropyl)amino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid, methyl ester (137)

Synthesized by method G in dichloromethane using 3-aminopropanol. Yield of title compound=75%.

### Example 114

### 3-[[(2,3-dihydroxypropyl)amino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid, methyl ester (138)

Synthesized by method G using 2,3-dihydroxypropylamine, and THF as a solvent. Yield of title compound=70%.

### Example 115

### 3-[[(5-hydroxy-3-oxapentyl)amino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid, methyl ester (139)

Synthesized by method G using 2-(2-aminoethoxy)ethanol, and dichloromethane as a solvent. Yield of title compound=85%.

### Example 116

### 3-[[(3,6-dioxaheptyl)amino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid, methyl ester (140)

Synthesized by method G using 2-(2-methoxyethoxy)ethylamine, and dichloromethane as a solvent. Yield=73%

### Example 117

### 3-[[(5-acetoxy-3-oxapentyl)amino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid, methyl ester (141)

Synthesized by method G using 2-(2-acetoxyethoxy)ethylamine, and dichloromethane as a solvent. Yield of title compound=72%.

### Example 118

### 3-[[(5-amino-3-oxapentyl)amino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid, methyl ester (142)

Synthesized by method G using 2-(2-aminoethoxy)ethylamine, and dichloromethane as a solvent. Yield of title compound=70%.

### Example 119

### 3-[[(5-N,N-dimethylamino-3-oxapentyl)amino]carbonyl]-13-(2-hydroxyethyl)-18-ethyl-7,8-dihydro-5-(2-methoxy-2-oxoethyl)-2,8,12,17-tetramethyl-21H, 23H-porphine-7-propionic acid, methyl ester (143)

Synthesized by Method G using [2-(2-aminoethoxy)ethyl]dimethylamine, and dichloromethane as a solvent. Yield of title compound=70%.

### Example 120

### 3-[[(6-hydroxy-4-(2-hydroxyethyl)-4-azahexyl)amino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid, methyl ester (144)

Synthesized by method G using N-(3-aminopropyl)diethanolamine, and dichloromethane as a solvent. Yield of title compound=84%.

### Example 121

### 3-[[(6-methoxy-4-(2-methoxyethyl)-4-azahexyl)amino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid, methyl ester (145)

Synthesized by method G using (3-aminopropyl)bis(2-methoxyethyl)amine, and dichloromethane as a solvent. Yield of title compound=82%.

### Example 122

### 3-[[(6-acetoxy-4-(2-acetoxyethyl)-4-azahexyl)amino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid, methyl ester (146)

Synthesized by method G using (3-aminopropyl)bis(2-acetoxyethyl)amine, and dichloromethane as a solvent. Yield of title compound=69%.

### Example 123

### 3-[[(8-hydroxy-3,6-dioxaoctyl)amino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid, methyl ester (147)

Synthesized by Method F using 2-[2-[2-aminoethoxy]ethoxy]ethanol, and dichloromethane as a solvent. Yield of title compound=74%.

### Example 124

### 3-[[(3,6,9-trioxadecyl)amino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid methyl ester (148)

Synthesized by Method G using 2-[2-(2-methoxyethoxy)ethoxy]ethylamine, and dichloromethane as a solvent. Yield of title compound=75%.

### 2-Desvinyl-2-(1-hydroxymethyl) Chlorin e6 amides

### Example 125

### 2-Desvinyl-2-(1-hydroxymethyl)pheophorbide methyl ester (4) (X═H, R═H)

Methyl pheophorbide (1.5 g) was dissolved in HBr/acetic acid (33%) (25 mL) with sonication and the solution was stirred overnight. Water (200 mL) was added and the pheophorbide precipitate was collected by filtration, washed with water (100 mL) and dried. The residue was dissolved in MeOH/CH(OMe)₃ (50 mL/20 mL) and water/sulfuric acid (6 mL/3 mL) was added. The solution was stirred for 2 hrs then poured into a NaHCO₃ solution (6 g/100 mL) with extensive stirring. The precipitated solid was collected by filtration, washed with water (50 mL) and dried. The compound was dissolved in dichloromethane and chromatographed on silica using 2-5% acetone/dichloromethane. The major gray fraction was collected and evaporated and precipitated from methanol/dichloromethane, filtered and dried. Yield of title compound=0.99 g.

The 2-desvinyl-2-(1-hydroxymethyl) chlorin e6 amide analogs were prepared using general method H.

### General Method H

2-Desvinyl-2-(1-hydroxymethyl) pheophorbide methyl ester (0.3 g) was dissolved in dichloromethane or THF (20 mL) and the reacting amine (0.6 g) was added. The reaction was stirred overnight at room temperature and the solvent removed by rotary evaporation. The-crude residue was dissolved in dichloromethane (2 mL) and chromatographed on silica using 1-5% methanol/dichloromethane as eluent. The major green fraction was collected (and chromatographed) and precipitated from hexane/dichloromethane. General yield of the compounds=60-80%.

### Example 126

### 3-[[methylamino]carbonyl]-13-(1-hydroxyethyl)-18-ethyl-7,8-dihydro-5-(2-methoxy-2-oxoethyl)-2,8,12,17-tetramethyl-21H, 23H-Porphine-7-propionic acid, methyl ester (149)

Synthesized according to method H using methylamine in a THF solution (2M). Yield of title compound=80%.

### Example 127

### 3-[[hexylamino]carbonyl]-13-(1-hydroxyethyl)-18-ethyl-7,8-dihydro-5-(2-methoxy-2-oxoethyl)-2,8,12,17-tetramethyl-21H, 23H-Porphine-7-propionic acid, methyl ester (150)

Synthesized by method H in dichloromethane using hexylamine, and dichloromethane as a solvent. Yield of title compound=80%.

### Example 128

### 3-[[(2-hydroxyethyl)amino]carbonyl]-13-(1-hydroxyethyl)-18-ethyl-7,8-dihydro-5-(2-methoxy-2-oxoethyl)-2,8,12,17-tetramethyl-21H, 23H-Porphine-7-propionic acid, methyl ester (151)

Synthesized by method H in dichloromethane using 2-aminoethanol, and dichloromethane as a solvent. Yield of title compound=85%.

### Example 129

### 3-[[(3-hydroxypropyl)amino]carbonyl]-13-(1-hydroxyethyl)-18-ethyl-7,8-dihydro-5-(2-methoxy-2-oxoethyl)-2,8,12,17-tetramethyl-21H, 23H-Porphine-7-propionic acid, methyl ester (152)

Synthesized by method H in dichloromethane using 3-aminopropanol, and dichloromethane as a solvent. Yield of title compound=85%.

### Example 130

### 3-[[(2,3-dihydroxypropyl)amino]carbonyl]-13-(1-hydroxyethyl)-18-ethyl-7,8-dihydro-5-(2-methoxy-2-oxoethyl)-2,8,12,17-tetramethyl-21H, 23H-Porphine-7-propionic acid, methyl ester (153)

Synthesized by method H using 2,3-dihydroxypropylamine, and THF as a solvent. Yield of title compound=70%.

### Example 131

### 3-[[(5-hydroxy-3-oxapentyl)amino]carbonyl]-13-(1-hydroxyethyl )-18-ethyl-7,8-dihydro-5-(2-methoxy-2-oxoethyl)-2,8,12,17-tetramethyl-21H, 23H-Porphine-7-propionic acid, methyl ester (154)

Synthesized by method H using 2-(2-aminoethoxy)ethanol, and dichloromethane as a solvent. Yield of title compound=85%.

### Example 132

### 3-[[(3,6-dioxaheptyl)amino]carbonyl]-13-(1-hydroxyethyl)-18-ethyl-7,8-dihydro-5-(2-methoxy-2-oxoethyl)-2,8,12,17-tetramethyl-21H, 23H-Porphine-7-propionic acid, methyl ester (155)

Synthesized by method H using 2-(2-methoxyethoxy)ethylamine, and dichloromethane as a solvent. Yield=73%

### Example 133

### 3-[[(5-acetoxy-3-oxapentyl)amino]carbonyl]-13-(1-hydroxyethyl)-18-ethyl-7,8-dihydro-5-(2-methoxy-2-oxoethyl)-2,8,12,17-tetramethyl-21H, 23H-Porphine-7-propionic acid, methyl ester (156)

Synthesized by method H using 2-(2-acetoxyethoxy)ethylamine, and dichloromethane as a solvent. Yield of title compound=72%.

### Example 134

### 3-[[(5-amino-3-oxapentyl)amino]carbonyl]-13-(1-hydroxyethyl)-18-ethyl-7,8-dihydro-5-(2-methoxy-2-oxoethyl)-2,8,12,17-tetramethyl-21H, 23H-Porphine-7-propionic acid, methyl ester (157)

Synthesized by Method H using 2-(2-aminoethoxy)ethylamine, and dichloromethane as a solvent. Yield of title compound=80%.

### Example 135

### 3-[[(5-N,N-dimethylamino-3-oxapentyl)amino]carbonyl]-13-(1-hydroxyethyl)-18-ethyl-7,8-dihydro-5-(2-methoxy-2-oxoethyl)-2,8,12,17-tetramethyl-21H, 23H-porphine-7-propionic acid, methyl ester (158)

Synthesized by Method H using [2-(2-aminoethoxy)ethyl]dimethylamine, and dichloromethane as a solvent. Yield of title compound=75%.

### Example 136

### 3-[[(6-hydroxy-4-(2-hydroxyethyl)-4-azahexyl)amino]carbonyl]-13-(1-hydroxyethyl)-18-ethyl-7,8-dihydro-5-(2-methoxy-2-oxoethyl)-2,8,12,17-tetramethyl-21H, 23H-Porphine-7-propionic acid, methyl ester (159)

Synthesized by method H using N-(3-aminopropyl)diethanolamine, and dichloromethane as a solvent. Yield of title compound=84%.

### Example 137

### 3-[[(6-methoxy-4-(2-methoxyethyl)-4-azahexyl)amino]carbonyl]-13-(2-hydroxyethyl)-18-ethyl-7,8-dihydro-5-(2-methoxy-2-oxoethyl)-2,8,12,17-tetramethyl-21H, 23H-Porphine-7-propionic acid, methyl ester (160)

Synthesized by method H using (3-aminopropyl)bis(2-methoxyethyl)amine, and dichloromethane as a solvent. Yield of title compound=82%.

### Example 138

### 3-[[(6-acetoxy-4-(2-acetoxyethyl)-4-azahexyl)amino]carbonyl]-13-(1-hydroxyethyl)-18-ethyl-7,8-dihydro-5-(2-methoxy-2-oxoethyl)-2,8,12,17-tetramethyl-21H, 23H-Porphine-7-propionic acid, methyl ester (161)

Synthesized by method H using (3-aminopropyl)bis(2-acetoxyethyl)amine, and dichloromethane as a solvent. Yield of title compound=72%.

### Example 139

### 3-[[(8-hydroxy-3,6-dioxaoctyl)amino]carbonyl]-13-(1-hydroxyethyl)-18-ethyl-7,8-dihydro-5-(2-methoxy-2-oxoethyl)-2,8,12,17-tetramethyl-21H, 23H-Porphine-7-propionic acid, methyl ester (162)

Synthesized by Method H using 2-[2-[2-aminoethoxy]ethoxy], and dichloromethane as a solvent. Yield of title compound=84%.

### Example 140

### 3-[[(3,6,9-trioxadecyl)amino]carbonyl]-13-(1-hydroxyethyl)-18-ethyl-7,8-dihydro-5-(2-methoxy-2-oxoethyl)-2,8,12,17-tetramethyl-21H, 23H-Porphine-7-propionic acid methyl ester (163)

Synthesized by Method H using 2-[2-(2-methoxyethoxy)ethoxy]ethylamine, and dichloromethane as a solvent. Yield of title compound=85%.

### 2-Desvinyl-2-(1-alkyloxyethyl) Chlorin e6 amides

### Example 141

### 2-Desvinyl-2-(1-heptyloxyethyl)pheophorbide methyl ester

Methyl pheophorbide (1.5 g) was dissolved in HBr/acetic acid (33%) (25 mL) with sonication and the solution was stirred for 2-3 hrs. The solvent was removed by rotoevaporation at high vacuum/low temperature and anhydrous heptanol (50 mL) was added. The solution was stirred at room temperature for 3 hrs making sure that the solid was dissolved. Water (500 mL) was added and the pheophorbide precipitate was collected by filtration, washed with water (100 mL) and dried. The residue was chromatographed on silica using 2-5% acetone/dichloromethane. The major gray fraction was collected and evaporated, precipitated from methanol/dichloromethane, filtered and dried. Yield of title compound=0.85 g.

### Example 142

### 2-Desvinyl-2-(1-hexyloxymethyl)pheophorbide methyl ester

The title compound was synthesized using the method described in Example 141, except that dry 1-hexanol was used instead of 1-pentanol. Yield=0.8 g.

### Example 143

### 2-Desvinyl-2-(1-methoxyethyl)pheophorbide methyl ester

The title compound was synthesized using the method described in Example 141, except that dry methanol was used instead of 1-pentanol. Yield=0.79 g.

### General Method I

### 2-Desvinyl-2-(1-alkoxyethyl)pheophorbide methyl ester ring opening Method

2-Desvinyl-2-(1-alkoxyethyl)pheophorbide methyl ester (0.1 g) was dissolved in dichloromethane (or THF) (5 mL) and the reacting amine was added (0.4 g). The solution was stirred at room temperature for 1 or 2 days (depending on the rate of the reaction) and once complete by TLC, the solvent was removed by rotary evaporation. The crude residue was chromatographed on silica using 2-10% methanol as eluent and the major fraction collected, evaporated and precipitated from methanol/dichloromethane or hexane/dichloromethane. Yield of the 2-Desvinyl-2-(1-alkoxyethyl) chlorin e6 amides was generally between 70-90%.

### Example 144

### 3-[[methylamino]carbonyl]-13-(1-methoxyethyl)-18-ethyl-7,8-dihydro-5-(2-methoxy-2-oxoethyl)-2,8,12,17-tetramethyl-21H, 23H-Porphine-7-propionic acid, methyl ester (164)

Synthesized according to method I using methylamine in a THF solution (2M) with stirring for 2 days. The compound was precipitated from dichloromethane/hexane. Yield of title compound=80%.

### Example 145

### 3-[[3-hydroxypropylamino]carbonyl]-13-(1-methoxyethyl)-18-ethyl-7,8-dihydro-5-(2-methoxy-2-oxoethyl)-2,8,12,17-tetramethyl-21H, 23H-Porphine-7-propionic acid, methyl ester (165)

Synthesized by method I in dichloromethane using 3-aminopropanol with stirring for 2 days. The compound was precipitated from dichloromethane/hexane. Yield of title compound=80%.

### Example 146

### 3-[[(5-hydroxy-3-oxapentyl)amino]carbonyl]-13-(1-methoxyethyl)-18-ethyl-7,8-dihydro-5-(2-methoxy-2-oxoethyl)-2,8,12,17-tetramethyl-21H, 23H-Porphine-7-propionic acid, methyl ester (166)

Synthesized by method I in dichloromethane using 2-(2-aminoethoxy)ethanol with stirring for 2 days. The compound was precipitated from dichloromethane/hexane. Yield of title compound=85%.

### Example 147

### 3-[[methylamino]carbonyl]-13-(1-heptyloxyethyl)-18-ethyl-7,8-dihydro-5-(2-methoxy-2-oxoethyl)-2,8,12,17-tetramethyl-21H, 23H-Porphine-7-propionic acid, methyl ester (167)

Synthesized according to method I using methylamine in a THF solution (2M) with stirring for 2 days. The compound was precipitated from dichloromethane/hexane. Yield of title compound=80%.

### Example 148

### 3-[[3-hydroxypropylamino]carbonyl]-13-(1-heptyloxyethyl)-18-ethyl-7,8-dihydro-5-(2-methoxy-2-oxoethyl)-2,8,12,17-tetramethyl-21H, 23H-Porphine-7-propionic acid, methyl ester (168)

Synthesized by method I in dichloromethane using 3-aminopropanol with stirring for 2 days. The compound was precipitated from dichloromethane/hexane. Yield of title compound=80%.

### Example 149

### 3-[[(5-hydroxy-3-oxapentyl)amino]carbonyl]-13-(1-heptyloxyethyl)-18-ethyl-7,8-dihydro-5-(2-methoxy-2-oxoethyl)-2,8,12,17-tetramethyl-21H, 23H-Porphine-7-propionic acid, methyl ester (169)

Synthesized by method I in dichloromethane using 2-(2-aminoethoxy)ethanol with stirring for 2 days. The compound was precipitated from dichloromethane/hexane. Yield of title compound=85%.

### Example 150

### 3-[[methylamino]carbonyl]-13-(1-hexyloxymethyl)-18-ethyl-7,8-dihydro-5-(2-methoxy-2-oxoethyl)-2,8,12,17-tetramethyl-21H, 23H-Porphine-7-propionic acid, methyl ester (170)

Synthesized according to method I using methylamine in a THF solution (2M) with stirring for 2 days. The compound was precipitated from dichloromethane/hexane. Yield of title compound=80%.

### Example 151

### 3-[[3-hydroxypropylamino]carbonyl]-13-(1-hexyloxymethyl)-18-ethyl-7,8-dihydro-5-(2-methoxy-2-oxoethyl)-2,8,12,17-tetramethyl-21H, 23H-Porphine-7-propionic acid, methyl ester (171)

Synthesized by method I in dichloromethane using 3-aminopropanol with stirring for 2 days. The compound was precipitated from dichloromethane/hexane. Yield of title compound=80%.

### Example 152

### 3-[[(5-hydroxy-3-oxapentyl)amino]carbonyl]-13-(1-hexyloxymethyl)-18-ethyl-7,8-dihydro-5-(2-methoxy-2-oxoethyl)-2,8,12,17-tetramethyl-21H, 23H-Porphine-7-propionic acid, methyl ester (172)

Synthesized by method I in dichloromethane using 2-(2-aminoethoxy)ethanol with stirring for 2 days. The compound was precipitated from dichloromethane/hexane. Yield of title compound=85%.

### Example 153

### 3-[[(5-N,N-dimethylamino-3-oxapentyl)amino]carbonyl]-13-(1-hexyloxymethyl)-18-ethyl-7,8-dihydro-5-(2-methoxy-2-oxoethyl)-2,8,12,17-tetramethyl-21H, 23H-porphine-7-propionic acid, methyl ester (173)

Synthesized by method I in dichloromethane using [2-(2-aminoethoxy)ethyl]dimethylamine with stirring for 2 days. The compound was precipitated from dichloromethane/hexane. Yield of title compound=75%.

### Example 154

### 3-[[(6-hydroxy-4-(2-hydroxyethyl)-4-azahexyl)amino]carbonyl]-13-(1-hexyloxymethyl)-18-ethyl-7,8-dihydro-5-(2-methoxy-2-oxoethyl)-2,8,12,17-tetramethyl-21H, 23H-Porphine-7-propionic acid, methyl ester (174)

Synthesized by method I using N-(3-aminopropyl)diethanolamine in a THF solution (2M) with stirring for 2 days. The compound was precipitated from dichloromethane/hexane. Yield of title compound=84%.

### Example 155

### 3-[[(8-hydroxy-3,6-dioxaoctyl)amino]carbonyl]-13-(1-hexyloxymethyl)-18-ethyl-7,8-dihydro-5-(2-methoxy-2-oxoethyl)-2,8,12,17-tetramethyl-21H, 23H-Porphine-7-propionic acid, methyl ester (175)

Synthesized by method I in dichloromethane using 2-[2-[2-methoxyethoxy)ethoxy]ethylamine with stirring for 2 days. The compound was precipitated from dichloromethane/hexane. Yield of title compound=84%.

### Example 156

### 3-[[(3,6,9-trioxadecyl)amino]carbonyl]-13-(1-hexyloxymethyl)-18-ethyl-7,8-dihydro-5-(2-methoxy-2-oxoethyl)-2,8,12,17-tetramethyl-21H, 23H-Porphine-7-propionic acid methyl ester (177)

Synthesized by method I in dichloromethane using 2-[2-(2-methoxyethoxy)ethoxy]ethylamine with stirring for 2 days. The compound was precipitated from dichloromethane/hexane. Yield of title compound=85%.

### 2-Desvinyl-2-((2-N,N-dimethylaminomethyl)vinyl) Chlorin e6 amides

### Example 157

### 2-Desvinyl-2-(2-N,N-dimethylaminomethyl)vinyl) pheophorbide methyl ester (6), X═H

Methyl pheophorbide (1.5 g) was dissolved in dichloromethane (200 mL, dry) and Eschenmoser's salt (4 g, fresh bottle) was added. The solution was stirred for 3 days under argon. Water was added (100 mL) and the organic layer was separated. The aqueous layer was washed with dichloromethane (50 mL) and the combined organic layers were evaporated to ˜50 mL. The solution was chromatographed on silica using 10% methanol/dichloromethane as solvent and the major product band collected. The solvent was removed by rotoevaporation and the solid was dried under vacuum. Yield of title compound=1.0 g.

### General method J

### 2-Desvinyl-2-(2-N,N-dimethylaminomethyl)vinyl) pheophorbide methyl ester ring opening method

2-Desvinyl-2-(2-N,N-dimethylaminomethyl)vinyl) pheophorbide methyl ester (0.1 g) was dissolved in dichloromethane (or THF) (5 mL) and the reacting amine was added (0.4 g). The solution was stirred at room temperature for 2-6 days (depending on the rate of the reaction) and once complete by TLC, the solvent was removed by rotary evaporation. The crude residue was chromatographed on silica using 2-10% methanol/dichloromethane/triethylamine (0.2%) as eluent and the major fraction was collected, evaporated and dried. Yield of the 2-Desvinyl-2-((2-N,N-dimethylaminomethyl)vinyl) chlorin e6 amides is generally between 70-90%.

### Example 158

### 3-[[methylamino]carbonyl]-13-((2-N,N-dimethylaminomethyl)vinyl))-18-ethyl-7,8-dihydro-5-(2-methoxy-2-oxoethyl)-2,8,12,17-tetramethyl-21H, 23H-Porphine-7-propionic acid, methyl ester (177)

Synthesized according to method J using methylamine in a THF solution (2M) with stirring for 4 days. Yield of title compound=80%.

### Example 159

### 3-[[3-hydroxypropylamino]carbonyl]-13-((2-N,N-dimethylaminomethyl)vinyl)) 18-ethyl-7,8-dihydro-5-(2-methoxy-2-oxoethyl)-2,8,12,17-tetramethyl-21H, 23H-Porphine-7-propionic acid, methyl ester (178)

Synthesized by method J in dichloromethane using 3-aminopropanol with stirring for 2 days. Yield of title compound=80%.

### Example 160

### 3-[[(5-hydroxy-3-oxa-pentyl)amino]carbonyl]-13-((2-N,N-dimethylaminomethyl)vinyl)-18-ethyl-7,8-dihydro-5-(2-methoxy-2-oxoethyl)-2,8,12,17-tetramethyl-21H, 23H-Porphine-7-propionic acid, methyl ester (179)

Synthesized by method J in dichloromethane using 2-(2-aminoethoxy)ethanol with stirring for 2 days. Yield of title compound=85%.

### Example 161

### 3-[[(5-N,N-dimethylamino-3-oxa-pentyl)amino]carbonyl]-13-((2-N,N-dimethylaminomethyl)vinyl)-18-ethyl-7,8-dihydro-5-(2-methoxy-2-oxoethyl)-2,8,12,17-tetramethyl-21H, 23H-porphine-7-propionic acid, methyl ester (180)

Synthesized by method J in dichloromethane using [2-(2-aminoethoxy)ethyl]dimethylamine with stirring for 2 days. Yield of title compound=75%.

### Example 162

### 3-[[(6-hydroxy-4-(2-hydroxyethyl)-4-azahexyl)amino]carbonyl]-13-((2-N,N-dimethylaminomethyl)vinyl)-18-ethyl-7,8-dihydro-5-(2-methoxy-2-oxoethyl)-2,8,12,17-tetramethyl-21H, 23H-Porphine-7-propionic acid, methyl ester (181)

Synthesized by method J in dichloromethane using N-(3-aminopropyl)diethanolamine with stirring for 3 days. Yield of title compound=84%.

### Example 163

### 3-[[(8-hydroxy-3,6-dioxaoctyl)amino]carbonyl]-13-((2-N,N-dimethylaminomethyl)vinyl)-18-ethyl-7,8-dihydro-5-(2-methoxy-2-oxoethyl)-2,8,12,17-tetramethyl-21H, 23H-Porphine-7-propionic acid, methyl ester (182)

Synthesized by method J in dichloromethane using 2-[2-[2-aminoethoxy]ethoxy]ethanol with stirring for 3 days. Yield of title compound=84%.

### Example 164

### 3-[[(3,6,9-trioxadecyl)amino]carbonyl]-13-((2-N,N-dimethylaminomethyl)vinyl)-18-ethyl-7,8-dihydro-5-(2-methoxy-2-oxoethyl)-2,8,12,17-tetramethyl-21H, 23H-Porphine-7-propionic acid methyl ester (183)

Synthesized by method J in dichloromethane using 2-[2-(2-methoxyethoxy)ethoxy]ethylamine with stirring for 4 days. Yield of title compound=85%.

### General Method K

### Zinc Metallation Procedure of Chlorin e6 Amides

To a solution of the chlorin e6 amide (0.1 g) in chloroform (20 mL) was added a solution of zinc acetate (0.2 g) in methanol (20 mL). The solution was refluxed for 0.5 hrs and the solvent was removed by rotary evaporation. The crude residue was dissolved in dichloromethane (100 mL), washed with water (100 mL), and the organic layer dried over sodium sulfate. After filtration, the organic layer was removed by rotary evaporation and dried and either precipitated with hexane/dichloromethane or used directly in biological studies. Using this method, compounds 184-264 were generated in quantitative yields from their free base precursors. Compounds 187, 196,198, 206, 212, and 223 were used directly without precipitation and the others were precipitated with hexane/dichloromethane.

### General Method L

### Gallium Metallation Procedure

Procedure A) Compounds not Bearing a Hydroxyl Group

To a solution of the chlorin e6 analog (0.2 g) in acetic acid (20 mL) was added Gallium acetylacetonate (0.2 g). The solution was refluxed for 1.5 hrs and the solvent was removed by rotary evaporation. The crude residue was dissolved in dichloromethane (100 mL), washed with water (100 mL), and 1N HCl (2×200 mL), and the organic layer was dried over sodium sulfate. After filtration, the organic layer was removed by rotary evaporation and the residue purified by column chromatography on silica using a mobile phase suitable for its purification (usually 2-10% methanol/dichloromethane). The compounds were then collected, evaporated and either precipitated with a suitable solvent (hexane/dichloromethane) or used directly.

Using this method, compounds 265, 266, 268, 269, 271 and 272 were generated in yields ˜60% from their free base precursors. Compound 268 was used directly without precipitation and the others were precipitated with hexane/dichloromethane.

Procedure B) Compounds Bearing a Hydroxyl Group

To a solution of the chlorin e6 analog (0.2 g) in acetic acid (20 mL) was added gallium acetylacetonate (0.2 g). The solution was refluxed for 1.5 hrs and the solvent removed by rotary evaporation. The crude residue was dissolved in dichloromethane (100 mL) and washed with water (100 mL) and the organic layer was dried over sodium sulfate. After filtration, the organic layer was removed by rotary evaporation. The gallium complex was dissolved in methanol (50 mL) and K₂CO₃(0.5 g) was added. The solution was stirred overnight and the solvent was removed by rotary evaporation. Dichloromethane (50 mL) was added, followed by water (100 mL) and the solution extracted with more dichloromethane (50 mL). The organic layers were washed with 1N HCl (2×200 mL), combined and dried, filtered and evaporated to dryness. The residue was chromatographed on silica using a mobile phase suitable for its purification (usually 10% methanol/dichloromethane). The compounds were then collected, and evaporated and either precipitated with hexane/dichloromethane or used directly in the biological studies.

Using this method compounds 267, 270, 273, 274, 275 and 276 were generated in yields ˜60% from their free base precursors. Compound 274 was used directly without precipitation, the others were precipitated with hexane/dichloromethane.

### Biological Evaluation of Photosensitizers

### Example 165

A) The Rat Choroidal Neovessel Model: Choroidal Neovessel Closure

The chlorin e6molecules were evaluated in a series of preclinical ocular neovessel models, corneal neovascularization, normal choriocapillaris vessels, and choroidal neovascularization.

Experimentally Induced Corneal Neovascularization

Corneal neovessels were experimentally induced in Sprague Dawley rats with an N-heptanol chemical scrub. The chemical scrub was used to remove the corneal epithelium and stem cells, allowing the neovessels to grow across the entire cornea. PDT was performed at approximately 3 weeks after the chemical scrub when the neovessels formed a uniform network across the cornea. The PDT treatment was applied to the corneal surface with a laser wavelength that was optimized for the given absorption spectrum. The laser energy was coupled through a slit lamp biomicroscope with a slit lamp adapter. A 3.0 mm spot size was used (Area=7.07 mm²). The light dose delivered was varied from 5-25 J/cm². The efficacy of neovessel closure was evaluated by measuring the area of treated cornea that remained neovessel-free out to 28 days following PDT. Accurate area measurements were taken using fluorescein angiography and measuring the area of neovessel-free cornea. Absence of fluorescein leakage in the treatment area demonstrated closure of the neovessels. The dosimetry and results of selected chlorin e6 molecules in this model are summarized in Table 16.

TABLE 16
A summary of the optimal drug dose and time interval for PDT
treatment of corneal neovessels induced by an n-heptanol scrub.
The light dose was 20 J/cm² at the corresponding
wavelength for optimal excitation of each photosensitizer. The
time interval (min) is the time between drug and light
administration.

| | | | | Extent of |
|---|---|---|---|---|
| | | | | neovessel |
| | | | Time | closure at 28 |
| | Excitation | Drug Dose | Interval | days after |
| Compound | Wavelength (nm) | (μmoles/kg) | (min) | treatment⁼ |
| Visudyne | 689 | 1.4 | 15 | 0 |
| 39 | 664 | 0.6 | 20 | 1 |
| 30 | 664 | 0.4 | 20 | 2 |
| 106 | 689 | 1.2 | 10 | 1 |
| 101 | 689 | 0.5 | 10 | 0 |
| 45 | 664 | 0.35 | 10 | 1 |
| 34 | 664 | 0.4 | 20 | 1 |
| 43 | 664 | 0.4 | 10 | 1 |

⁼Grading scale based on corneal fluorescein angiography: 0 = 0-0.2 mm², 1 = 0.21-1.0 mm², 2 = 1.1-2.0 mm², 3 = 2.1-3.0 mm², 4 = 3.1-7.0 mm², 5 = >7.0 mm²

Normal Choriocapillaris Rabbit Model

Selected chlorin e6 molecules were also evaluated in a normal choriocapillaris model in the pigmented rabbit. This model used the choriocapillaris as a surrogate for neovasculature to demonstrate PDT efficacy and longevity of vessel closure in the posterior segment of the eye. The selected photosensitizers were administered intravenously at varying drug doses, the light dose was set constant at 20 J/cm², and the time interval was varied from 5-30 minutes between drug and light administration. Two PDT treatment areas were placed on the fundus of each eye in each rabbit. Fluorescein angiography was used to evaluate vessel closure following PDT out to 28 days. The dosimetry and efficacy results of these compounds are summarized in Table 17.

TABLE 17
Optimal dosimetry and results summarizing the closure
of the choriocapillaris at 28 days following PDT. The
light dose for all treatments was 20 J/cm².

| | Drug Dose | Time Interval | Closure at 28 |
|---|---|---|---|
| Photosensitizer | (μmoles/kg) | (min) | Days⁼ |
| Visudyne | 1.4 | 5-10 | 4 |
| 39 | 0.9 | 5-30 | 4 |
| 30 | 0.5 | 5-20 | 3 |
| 106 | 2.8 | 5-30 | 4 |
| 45 | 0.35 | 10 | 3 |
| 34 | 0.7 | 5-30 | 3 |

⁼Grading scale based on fluorescein angiography: 1 = 0-25%, 2 = 26-50%, 3 = 51-75%, 4 > 75%

Experimentally Induced Choroidal Neovascularization

Two of the chlorin e6molecules, (39) and (106), were evaluated in a laser-induced choroidal neovascularization (CNV) model in rats. Laser photocoagulation was used to stimulate choroidal neovessel growth on the fundus of the rat (E. T. Dobi, C. Puliafito, M. A. Destro, “A new model of experimental choroidal neovascularization in the rat”, Arch. Ophthalmol. 1989; 107: 264-269). The PDT treatments were performed approximately 4 weeks after the laser photocoagulation, which was when the choroidal neovascularization lesions were fully developed. The lesions were PDT treated using a 1.2 mm spot which covered the entire CNV lesion. Fluorescein angiography and histopathology were used to evaluate the CNV closure. Initial flush of the fluorescein angiography showed that molecule (39) (0.8 & 1.6 μmoles/kg, 10-20 minutes time interval post injection) closed the CNV lesion at 7 days after PDT. Molecule (106) (0.75 μmoles/kg, 20 minutes time interval post injection) also demonstrated CNV closure at 7 days post PDT based on fluorescein angiography.

In comparison, Visudyne also showed CNV closure at 7 days post treatment at a drug dose of 1.4 μmoles/kg, with light treatment 10-20 minutes post injection. However, CNV closure at 28 days post treatment at a drug dose of 1.4 μmoles/kg was only observed with light treatment 20 minutes post injection and not at 10 minutes.

In summary, the pharmacological properties of the novel compounds according to the invention are substantially different from those of existing photosensitizers described to date in the literature. In particular, the compounds investigated possess the following properties.
(I) They show short periods (<24 hrs) of skin photosensitivity.
(II) Several candidates are equal to or more effective at sustaining choriocapillaris in the rabbit at 28 days following PDT as compared to the commercial drug Visudyne, without retinal damage.
(III) They are made in excellent yield from readily available plant chlorophylls.
(IV) They demonstrate equal or superior results in closing choriocapillaris in rabbits without adjacent retinal damage compared to Visudyne.
(V) Upon light activation, the photodynamic effect is localized to the treatment zone of the vascular vessel, while sparing underlying tissues including the retina and pigment epithelium.
(VI) They may be suitable for other diseases in photodynamic therapy, including neovessel diseases, cancer, ophthalmic diseases, immune diseases and the like.

1. Compounds of formula I: 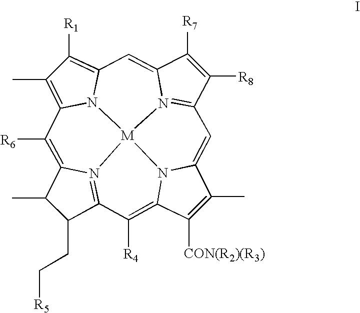
wherein:
R₁, R₅, R₆, R₇, and R₈ can be the same or different and are selected from the group consisting of:
H, halogen, substituted or unsubstituted C1-C20 alkyl, heteroalkyl, haloalkyl, heterohaloalkyl, cyclic alkyl, aryl, substituted aryl, alkenyl, haloalkenyl, substituted alkenyl, alkynyl, substituted alkynyl, amide, ester, ethers, polyethers, alkoxy group, aryloxy group, haloalkoxy group, amino group, alkylcarbonyloxy group, alkoxycarbonyl group, aryloxycarbonyl group, azo group, arylcarbonyloxy group, alkoxycarbonyloxy group, aryloxycarbonyloxy group, sulfinyl group, sulfonyl group, silil group, carbamoyl group, heterocyclic group, nitro group, nitroso group, formyloxy group, isocyano group, cyanate group, isocyanate group, thiocyanate group, isothiocyanate group, N(alkyl)₂, N(aryl)₂, CH═CH(aryl), CH═CHCH₂N(CH₃)₂, CH═CHCH₂N⁺(CH₃)₃A, CH═N(alkyl)₂⁺A, N(alkyl)₃⁺A, CN, OH, CHO, COCH₃, CO(alkyl), CO₂H, CO₂Na, CO₂K, CH(CH₃)OH, CH(CH₃)O-alkyl, CH(CH₃)O-alkoxy, CH(CH₃)O-aryl, CH(CH₃)NH-alkyl, CH(CH₃)NH-cycloalkyl, CH(CH₃)NH-heteroalkyl, CH(CH₃)NH-heteroalkoxy, CH(CH₃)-(amino acid), CH(CH₃)-(amino acid ester), CH(CH₃)-(amino acid amide), C(X)₂C(X)₃, CH═NR₉, NHCOCH₂N(CH₃)₂, NHCOCH₂N(CH₃)₃⁺A, NHCOCH₂-(pyridinium)⁺A, (CH₂)ₙO-alkoxy, and (CH₂)ₙO-alkyl, where X is selected from H and halogen, R₉ is selected from OH, O-alkyl, O-ether, and O-alkylamino, n is an integer ranging from 0 to 8, and A is a charge balancing ion;
CO₂R₁₀, where R₁₀ is selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons;
(CH₂)ₙOH and (CH₂)ₙOR₁₁, where R₁₁ is selected from alkyl, haloalkyl, heteroalkyl, haloheteroalkyl, aryl, heteroaryl, heterocycle, a protecting group, a mono-, di- or polyhydroxyalkyl residue, a mono-, di-, or polyhydroxyaryl residue, and a functional group of less than about 100,000 daltons, and n is an integer ranging from 0 to 4;
(CH₂)ₙCO₂R₁₂, (CHX)ₙCO₂R₁₂, and (CX₂)ₙCO₂R₁₂, where X is selected from OH, OR₁₃, or a halogen, and R₁₂ and R₁₃ can be the same or different and are selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons, and n is an integer ranging from 1 to 4;
CONHNH(R₁₄), CO(R₁₄), CON(R₁₄)₂, CON(R₁₄)(R₁₅), (CH₂)ₙCON(R₁₄)₂, (CH₂)ₙCOR₁₄, (CH₂)ₙCON(R₁₄)(R₁₅), (CX₂)ₙCONH(R₁₄), (CX₂)ₙCON(R₁₄)₂, (CX₂)ₙCON(R₁₄)(R₁₅), (CX₂)ₙCOR₁₄, (CH₂)ₙCONHNH(R₁₄), (CX₂)ₙCONHNH(R₁₄), (CHX)ₙCONH(R₁₅), (CHX)ₙCONHNH(R₁₄), (CHX)ₙCO(R₁₄), (CHX)ₙCON(R₁₄)₂, and (CHX)ₙCON(R₁₄)(R₁₅), where X is selected from OH, OR₁₆, or a halogen, and R₁₄, R₁₅ and R₁₆ can be the same or different and are selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
CONH(R₁₄) and (CH₂)ₙCONH(R₁₄), where R₁₄ is selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
S(R₁₇), CH(CH₃)S(R₁₇), (CH₂)ₙS(R₁₇), (CH₂)ₙNH(R₁₇), (CH₂)ₙNHNH(R₁₇), (CH₂)ₙN(R₁₇)₂, (CH₂)ₙN(R₁₇)(R₁₈), (CH₂)ₙN(R₁₇)(R₁₈)(R₁₉)⁺A, CH═N(R₁₇), CH═NN(R₁₇)(R₁₈), and amino acids containing —NH(R₁₇) or —N(R₁₇)(R₁₈), where R₁₇, R₁₈ and R₁₉ can be the same or different and are selected from H, OH, O-alkyl, NH₂, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, where R₁₇, R₁₈ and R₁₉ may together possess the atoms necessary to constitute an aromatic ring system, n is an integer ranging from 0 to 4, and A is a physiologically acceptable counter ion;
(CH₂)ₙOPO(OR₂₀)₂ and (CH₂)ₙPO(OR₂₀)₂, where R₂₀ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and n is an integer ranging from 0 to 4;
(CH₂)ₙNHCOR₂₁, and (CH₂)ₙNHNHCOR₂₁, where R₂₁ is selected from a straight or branched chain C1-C20 alkyl, haloalkyl, heteroalkyl, haloheteroalkyl, aryl, heteroaryl, heterocycle, and a functional group of less than about 100,000 daltons, and n is an integer ranging from 0 to 4;
SO₃R₂₂, SO₂NHR₂₂, SO₂N(R₂₂)₂, SO₂NHNHR₂₂, SO₂R₂₂, (CH₂)ₙSO₂NHR₂₂, (CH₂)ₙSO₂N(R₂₂)₂, (CH₂)ₙSO₂NHNHR₂₂, and (CH₂)ₙSO₂R₂₂, where R₂₂ is selected from H, OH, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and NHR₂₂ can constitute an amino acid, an amino acid salt, an amino acid ester residue, or an amino acid amide residue, and n is an integer ranging from 0 to 4;
aryl or substituted aryl, which may bear one or more substituents with a molecular weight of less than or equal to about 100,000 daltons;
R₂ and R₃ can be the same or different and are selected from H, C1-C20 alkyl, C1-C20 cyclic alkyl, aryl, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN((CH₂)ₘN(CH₃)₂)₂, (CH₂)ₙO-haloalkyl, (CH₂)ₙN((CH₂)ₘN(CH₃)₃⁺A)₂, ((CH₂)ₙO)ₘ((CH₂)_{Q}O)COCH₃, a mono-, di-, or polyhydroxyaryl residue, a mono-, di-, or polyetheralkyl residue, and a mono-, di-, or polyetheraryl residue, and Q, n and m are integers ranging from 0 to 10,000 and A is a physiologically acceptable counter ion;
R₄ is selected from:
(CH₂)ₙCO₂R₂₃, (CHX)ₙCO₂R₂₃, and (CX₂)ₙCO₂R₂₃, where X is selected from OH, OR₂₄, SR₂₄, and a halogen, and R₂₃ and R₂₄ can be the same or different and are selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons, and n is an integer ranging from 1 to 4;
(CH₂)ₙCON(R₂₅)₂, (CH₂)ₙCOR₂₅, (CH₂)ₙCON(R₂₅)(R₂₆), (CX₂)ₙCONH(R₂₅), (CX₂)ₙCON(R₂₅)₂, (CX₂)ₙCON(R₂₅)(R₂₆), (CX₂)ₙCOR₂₅, (CH₂)ₙCONHNH(R₂₅), (CX₂)ₙCONHNH(R₂₅), (CHX)ₙCONH(R₂₅), (CHX)ₙCONHNH(R₂₅), (CHX)ₙCO(R₂₅), (CHX)ₙCON(R₂₅)₂, and (CHX)ₙCON(R₂₅)(R₂₆), where X is selected from OH, OR₂₇, SR₂₇, and a halogen, and R₂₅, R₂₆ and R₂₇ can be the same or different and are selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
(CH₂)ₙCONH(R₂₅), where R₂₅ is selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
(CH₂)ₙS(R₂₈), (CH₂)ₙNH(R₂₈), (CH₂)ₙNHNH(R₂₈), (CH₂)ₙN(R₂₈)₂, (CH₂)ₙN(R₂₈)(R₂₉), (CH₂)ₙN(R₂₈)(R₂₉)(R₃₀)⁺A, amino acids containing —NH(R₂₈), amino acid esters containing —NH(R₂₈), and amino acid amides containing —NH(R₂₈), where R₂₈, R₂₉ and R₃₀ can be the same or different and are selected from H, OH, NH₂, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, where R₂₈, R₂₉ and R₃₀ together may possess the atoms necessary to constitute an aromatic ring system, n is an integer ranging from 1 to 4, and A is a physiologically acceptable counter ion;
(CH₂)ₙOPO(OR₃₁)₂ and (CH₂)ₙPO(OR₃₁)₂, where R₃₁ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and n is an integer ranging from 0 to 4;
(CH₂)ₙOH and (CH₂)ₙOR₃₂, where R₃₂ is selected from alkyl, haloalkyl, heteroalkyl, haloheteroalkyl, aryl, heteroaryl, heterocycle, a protecting group, a mono-, di- or polyhydroxyalkyl residue, a mono-, di-, or polyhydroxyaryl residue, and a functional group of less than about 100,000 daltons, and n is an integer ranging from 0 to 4;
(CH₂)ₙNHCOR₃₃ and (CH₂)ₙNHNHCOR₃₃, where R₃₃ is selected from a straight or branched chain C1-C20 alkyl, OH, haloalkyl, heteroalkyl, haloheteroalkyl, aryl, heteroaryl, heterocycle, and a functional group of less than about 100,000 daltons, and n is an integer ranging from 1 to 4;
(CH₂)ₙSO₂NHR₃₄, (CH₂)ₙSO₂N(R₃₄)₂, (CH₂)ₙSO₂NHNHR₃₄, (CH₂)ₙSO₂R₃₄, (CH₂)ₙOSO₂NHR₃₄, (CH₂)ₙOSO₂N(R₃₄)₂, (CH₂)ₙOSO₂NHNHR₃₄, and (CH₂)ₙOSO₃R₃₄, where R₃₄ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, NHR₃₄ can constitute an amino acid, an amino acid salt, an amino acid ester residue, or an amino acid amide residue, and n is an integer ranging from 1 to 4; and aryl or substituted aryl, which may bear one or more substituents with a molecular weight of less than or equal to about 100,000 daltons;
with the proviso that when R₁ is vinyl, R₂ is H, R₄ is CH₂CO₂CH₃, R₅ is CO₂H, R₆ is H, R₇ is CH₃, and R₈ is CH₂CH₃, R₃ cannot be selected from (CH₂)₂CH₃, CH₂CH₃, CH(CH₃)₂, (CH₂)₃CH₃, CH₂-phenyl, and cyclohexyl; and wherein M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

2. A pharmaceutical composition comprising an effective diagnostic or therapeutic amount of the compound of claim 1, together with at least one pharmaceutically acceptable carrier or excipient.

3. The pharmaceutical composition according to claim 2 used to treat ophthalmic diseases.

4. The pharmaceutical composition of claim 3, wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

5. The pharmaceutical composition according to claim 2 used to treat cardiovascular diseases.

6. The pharmaceutical composition according to claim 2 used to treat skin diseases.

7. The pharmaceutical composition according to claim 2 used to treat cancer or malignant diseases.

8. Compounds of the following formula: 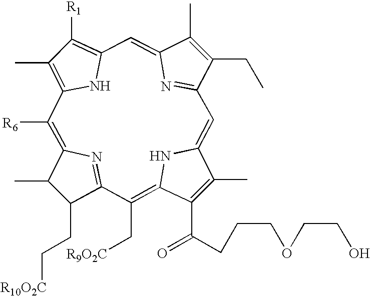
wherein:
R₁ is selected from H, CH₃, CH₂CH₃, CH═CH₂, CH₂OH, CH₂OAc, CH₂O-alkyl, CH₂O-alkoxy, CH═CHCH₂N(CH₃)₂, CH═CHCH₂N(CH₃)₃⁺A⁻, COCH₃, CHO, CH(OH)CH₃, CH(O-alkyl)CH₃, CH(O-alkoxy)CH₃, CH₂CH₂O-alkyl, CH₂CH₂O-alkoxy, and CH₂CH₂OAc, where A is a charge balancing ion;
R₆ is selected from H, halogen, and methyl;
R₉ is selected from alkyl, aryl, hydroxyalkyl, H, and a physiologically acceptable counter ion; and
R₁₀ is selected from alkyl, aryl, H, and a physiologically acceptable counter ion;
or a pharmaceutically acceptable salt, prodrug, solvate, or metabolite thereof.

9. A pharmaceutical composition comprising an effective diagnostic or therapeutic amount of the compound of claim 8, together with at least one pharmaceutically acceptable carrier or excipient.

10. The pharmaceutical composition according to claim 9 used to treat ophthalmic diseases.

11. The pharmaceutical composition of claim 10, wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

12. The pharmaceutical composition according to claim 9 used to treat cancer or malignant diseases.

13. The pharmaceutical composition according to claim 9 used to treat cardiovascular diseases.

14. The pharmaceutical composition according to claim 9 used to treat skin diseases.

15. Compounds of formula I: 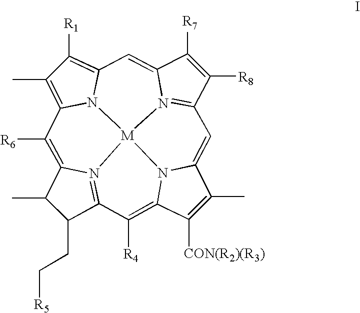
wherein:
R₁, R₇, and R₈ can be the same or different and are selected from vinyl, hydroxyalkyl, alkylether, and CH₂CH₂OCOCH₃;
R₆ is selected from methyl, halogen, CH═CHCHO, CH═CHCH₂OH, CH═CHCH₂O-alkyl, and CH═CHCH₂O-alkylether;
R₄ is selected from:
(CH₂)ₙCO₂R₉, (CHX)ₙCO₂R₉, and (CX₂)ₙCO₂R₉, where X is selected from OH, OR₁₀, SR₁₀, and a halogen, and R₉ and R₁₀ can be the same or different and are selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons, and n is an integer ranging from 1 to 4;
(CH₂)ₙCON(R₁₁)₂, (CH₂)ₙCOR₁₁, (CH₂)ₙCON(R₁₁)(R₁₂), (CX₂)ₙCONH(R₁₁), (CX₂)ₙCON(R₁₂)₂, (CX₂)ₙCON(R₁₁)(R₁₂), (CX₂)ₙCOR₁₁, (CH₂)ₙCONHNH(R₁₁), (CX₂)ₙCONHNH(R₁₁), (CHX)ₙCONH(R₁₁), (CHX)ₙCONHNH(R₁₁), (CHX)ₙCO(R₁₁), (CHX)ₙCON(R₁₁)₂, and (CHX)ₙCON(R₁₁)(R₁₂), where X is selected from OH, OR₁₃, SR₁₃, and a halogen, and R₁₁, R₁₂ and R₁₃ can be the same or different and are selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
(CH₂)ₙCONH(R₁₁), where R₁₁ is selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
(CH₂)ₙS(R₁₄), (CH₂)ₙNH(R₁₄), (CH₂)ₙNHNH(R₁₄), (CH₂)ₙN(R₁₄)₂, (CH₂)ₙN(R₁₄)(R₁₅), (CH₂)ₙN(R₁₄)(R₁₅)(R₁₆)⁺A, amino acids containing —NH(R₁₄), amino acid esters containing —NH(R₁₄), and amino acid amides containing —NH(R₁₄), where R₁₄, R₁₅ and R₁₆ can be the same or different and are selected from H, OH, NH₂, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, where R₁₄, R₁₅ and R₁₆ together may possess the atoms necessary to constitute an aromatic ring system, n is an integer ranging from 1 to 4, and A is a physiologically acceptable counter ion;
(CH₂)ₙOPO(OR₁₇)₂ and (CH₂)ₙPO(OR₁₇)₂, where R₁₇ is selected from H, OH, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and n is an integer ranging from 0 to 4;
(CH₂)ₙNHCOR₁₈ and (CH₂)ₙNHNHCOR₁₈, where R₁₈ is selected from a straight or branched chain C1-C20 alkyl, haloalkyl, heteroalkyl, haloheteroalkyl, aryl, heteroaryl, heterocycle, and a functional group of less than about 100,000 daltons, and n is an integer ranging from 0 to 4;
(CH₂)ₙSO₂NHR₁₉, (CH₂)ₙSO₂N(R₁₉)₂, (CH₂)ₙSO₂NHNHR₁₉, (CH₂)ₙSO₂R₁₉, (CH₂)ₙOSO₂NHR₁₉, (CH₂)ₙOSO₂N(R₁₉)₂, (CH₂)ₙOSO₂NHNHR₁₉, and (CH₂)ₙOSO₃R₁₉, where R₁₉ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and NHR₁₉ can constitute an amino acid, an amino acid salt, an amino acid ester residue, or an amino acid amide residue, and n is an integer between 0 and 4;
(CH₂)ₙOH and (CH₂)ₙOR₂₀ , where R₂₀ is selected from alkyl, haloalkyl, heteroalkyl, haloheteroalkyl, aryl, heteroaryl, heterocycle, a protecting group, a mono-, di- or polyhydroxyalkyl residue, a mono-, di-, or polyhydroxyaryl residue, and a functional group of less than about 100,000 daltons, and n is an integer ranging from 0 to 4; and
aryl or substituted aryl, which may bear one or more substituents with a molecular weight of less than or equal to about 100,000 daltons;
R₂ and R₃ can be the same or different and are selected from H, alkyl, cycloalkyl, aryl, (CH₂)ₙO-alkyl, (CH₂)ₙOCOCH₃, CH₂CH(OH)CH₂OH, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, ((CH₂)ₙO)ₘ(CH₂)_{Q}OH, ((CH₂)ₙO)ₘ(CH₂O)_{Q}COCH₃, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN((CH₂)ₘN(CH₃)₂)₂, (CH₂)ₙN((CH₂)ₘN(CH₃)₃⁺A)₂, ((CH₂)ₙO)ₘ(CH₂)_{Q}OH, ((CH₂)ₙO)ₘ(CH₂O)_{Q}COCH₃, (CH₂)ₙO-haloalkyl, (CH₂)ₙN((CH₂)ₘO-alkyl)₂, (CH₂)ₙN((CH₂)ₘO-alkylether)₂, CH₂CH(OAc)CH₂OAc, an alkylphosphate residue, an alkylsulfonic acid residue, an alkylsulfonic ester or amide reside, an alkylmorpholine residue, an alkylheterocyclic residue, an alkylthiol residue, a mono-, di-, or polyhydroxyaryl residue, a mono-, di-, or polyetheralkyl residue, and a mono-, di-, or polyetheraryl residue, wherein Q, n and m may be the same or different and are integers ranging from 0 to 10,000, and A is a charge balancing ion; and
M is selected from 2H, a metal cation, or photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

16. A pharmaceutical composition comprising an effective diagnostic or therapeutic amount of the compound of claim 15, together with at least one pharmaceutically acceptable carrier or excipient.

17. The pharmaceutical composition according to claim 16 used to treat ophthalmic diseases.

18. The pharmaceutical composition of claim 17, wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

19. The pharmaceutical composition according to claim 16 used to treat cancer or malignant diseases.

20. The pharmaceutical composition according to claim 16 used to treat cardiovascular diseases.

21. The pharmaceutical composition according to claim 16 used to treat skin diseases.

22. Compounds of formula I: 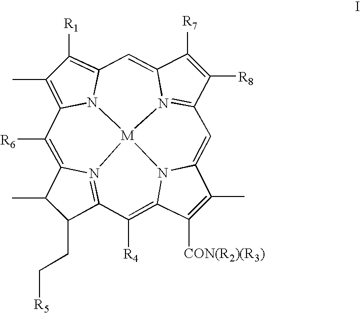
wherein:
R₁, R₅, R₆, R₇, and R₈ can be the same or different and are selected from the group consisting of:
H, halogen, substituted or unsubstituted C1-C20 alkyl, heteroalkyl, haloalkyl, heterohaloalkyl, cyclic alkyl, aryl, substituted aryl, alkenyl, haloalkenyl, substituted alkenyl, alkynyl, substituted alkynyl, amide, ester, ethers, polyethers, alkoxy group, aryloxy group, haloalkoxy group, amino group, alkylcarbonyloxy group, alkoxycarbonyl group, aryloxycarbonyl group, azo group, arylcarbonyloxy group, alkoxycarbonyloxy group, aryloxycarbonyloxy group, sulfinyl group, sulfonyl group, silil group, carbamoyl group, heterocyclic group, nitro group, nitroso group, formyloxy group, isocyano group, cyanate group, isocyanate group, thiocyanate group, isothiocyanate group, N(alkyl)₂, N(aryl)₂, CH═CH(aryl), CH═CHCH₂N(CH₃)₂, CH═CHCH₂N⁺(CH₃)₃A, CH═N(alkyl)₂⁺A, N(alkyl)₃⁺A, CN, OH, CHO, COCH₃, CO(alkyl), CO₂H, CO₂Na, CO₂K, CH(CH₃)OH, CH(CH₃)O-alkyl, CH(CH₃)O-alkoxy, CH(CH₃)O-aryl, CH(CH₃)NH-alkyl, CH(CH₃)NH-cycloalkyl, CH(CH₃)NH-heteroalkyl, CH(CH₃)NH-heteroalkoxy, CH(CH₃)-(amino acid), CH(CH₃)-(amino acid ester), CH(CH₃)-(amino acid amide), C(X)₂C(X)₃, CH═NR₉, NHCOCH₂N(CH₃)₂, NHCOCH₂N(CH₃)₃⁺A, NHCOCH₂-(pyridinium)⁺A, (CH₂)ₙO-alkoxy, and (CH₂)ₙO-alkyl, where X is selected from H and halogen, R₉ is selected from OH, O-alkyl, O-ether, and O-alkylamino, n is an integer ranging from 0 to 8, and A is a charge balancing ion;
CO₂R₁₀, where R₁₀ is selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons;
(CH₂)ₙOH and (CH₂)ₙOR₁₁, where R₁₁ is selected from alkyl, haloalkyl, heteroalkyl, haloheteroalkyl, aryl, heteroaryl, heterocycle, a protecting group, a mono-, di- or polyhydroxyalkyl residue, a mono-, di-, or polyhydroxyaryl residue, and a functional group of less than about 100,000 daltons, and n is an integer ranging from 0 to 4;
(CH₂)ₙCO₂R₁₂, (CHX)ₙCO₂R₁₂, and (CX₂)ₙCO₂R₁₂, where X is selected from OH, OR₁₃, and halogen, and R₁₂ and R₁₃ can be the same or different and are selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons, and n is an integer ranging from 1 to 4;
CONHNH(R₁₄), CO(R₁₄), CON(R₁₄)₂, CON(R₁₄)(R₁₅), (CH₂)ₙCON(R₁₄)₂, (CH₂)ₙCOR₁₄, (CH₂)ₙCON(R₁₄)(R₁₅), (CX₂)ₙCONH(R₁₄), (CX₂)ₙCON(R₁₄)₂, (CX₂)ₙCON(R₁₄)(R₁₅), (CX₂)ₙCOR₁₄, (CH₂)ₙCONHNH(R₁₄), (CX₂)ₙCONHNH(R₁₄), (CHX)ₙCONH(R₁₅), (CHX)ₙCONHNH(R₁₄), (CHX)ₙCO(R₁₄), (CHX)ₙCON(R₁₄)₂, and (CHX)ₙCON(R₁₄)(R₁₅), where X is selected from OH, OR₁₆, and halogen, and R₁₄, R₁₅ and R₁₆ can be the same or different and are selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
CONH(R₁₄) and (CH₂)ₙCONH(R₁₄), where R₁₄ is selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
S(R₁₇), CH(CH₃)S(R₁₇), (CH₂)ₙS(R₁₇), (CH₂)ₙNH(R₁₇), (CH₂)ₙNHNH(R₁₇), (CH₂)ₙN(R₁₇)₂, (CH₂)ₙN(R₁₇)(R₁₈), —(CH₂)ₙN(R₁₇)(R₁₈)(R₁₉)⁺A, CH═N(R₁₇), CH═NN(R₁₇)(R₁₈), and amino acids containing —NH(R₁₇) or —N(R₁₇)(R₁₈), where R₁₇, R₁₈ and R₁₉ can be the same or different and are selected from H, OH, O-alkyl, NH₂, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, where R₁₇, R₁₈ and R₁₉ may together possess the atoms necessary to constitute an aromatic ring system, n is an integer ranging from 0 to 4, and A is a physiologically acceptable counter ion;
(CH₂)ₙOPO(OR₂₀)₂ and (CH₂)ₙPO(OR₂₀)₂, where R₂₀ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and n is an integer ranging from 0 to 4;
(CH₂)ₙNHCOR₂₁ and (CH₂)ₙNHNHCOR₂₁, where R₂₁ is selected from a straight or branched chain C1-C20 alkyl, haloalkyl, heteroalkyl, haloheteroalkyl, aryl, heteroaryl, heterocycle, and a functional group of less than about 100,000 daltons, and n is an integer ranging from 0 to 4;
SO₃R₂₂, SO₂NHR₂₂, SO₂N(R₂₂)₂, SO₂NHNHR₂₂, SO₂R₂₂, (CH₂)ₙSO₂NHR₂₂, (CH₂)ₙSO₂N(R₂₂)₂, (CH₂)ₙSO₂NHNHR₂₂, and (CH₂)ₙSO₂R₂₂, where R₂₂ is selected from H, OH, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, NHR₂₂ can constitute an amino acid, an amino acid salt, an amino acid ester residue, or an amino acid amide residue, and n is an integer ranging from 0 to 4;
aryl or substituted aryl, which may bear one or more substituents with a molecular weight of less than or equal to about 100,000 daltons;
R₂ and R₃ can be the same or different and are selected from C1-C20 alkyl, C1-C20 cyclic alkyl, aryl, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN((CH₂)ₘN(CH₃)₂)₂, (CH₂)ₙO-haloalkyl, (CH₂)ₙN((CH₂)ₘN(CH₃)₃)₃⁺A)₂, ((CH₂)ₙO)ₘ((CH₂)_{Q}O)COCH₃, a mono-, di, or polyhydroxyalkyl residue, a mono, di, or polyhydroxyaryl residue, a mono-, di, or polyetheralkyl residue, and a mono-, di-, or polyetheraryl residue, where Q, n, and m are integers ranging from 0 to 10,000, and A is a physiologically acceptable counter ion;
R₄ is selected from:
(CH₂)ₙCO₂R₂₃, (CHX)ₙCO₂R₂₃, and (CX₂)ₙCO₂R₂₃, where X is selected from OH, OR₂₄, SR₂₄, and a halogen, and R₂₃ and R₂₄ can be the same or different and are selected from H, a physiologically acceptable counter ion, a straight or branched chain C1-C20 alkyl, haloakyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons, and n is an integer ranging from 1 to 4;
(CH₂)ₙCON(R₂₅)₂, (CH₂)ₙCOR₂₅, (CH₂)ₙCON(R₂₅)(R₂₆), (CX₂)ₙCONH(R₂₅), (CX₂)ₙCON(R₂₅)₂, (CX₂)ₙCON(R₂₅)(R₂₆), (CX₂)ₙCOR₂₅, (CH₂)ₙCONHNH(R₂₅), (CX₂)ₙCONHNH(R₂₅), (CHX)ₙCONH(R₂₅), (CHX)ₙCONHNH(R₂₅), (CHX)ₙCO(R₂₅), (CHX)ₙCON(R₂₅)₂, and (CHX)ₙCON(R₂₅)(R₂₆), where X is selected from OH, OR₂₇, SR₂₇, and a halogen, and R₂₅, R₂₆ and R₂₇ can be the same or different and are selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
(CH₂)ₙCONH(R₂₅), where R₂₅ is selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
(CH₂)ₙS(R₂₈), (CH₂)ₙNH(R₂₈), (CH₂)ₙNHNH(R₂₈), (CH₂)ₙN(R₂₈)₂, (CH₂)ₙN(R₂₈)(R₂₉ (CH₂)ₙN(R₂₈)(R₂₉)(R₃₀)⁺A, amino acids containing —NH(R₂₈), amino acid esters containing —NH(R₂₈), and amino acid amides containing —NH(R₂₈), where R₂₈, R₂₉ and R₃₀ can be the same or different and are selected from H, OH, NH₂, acetyl, a straight or branched chain C1-C20 alkyl, haloalkyl, heteroalkyl, haloheteroalkyl, aryl, heteroaryl, heterocycle, mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, where R₂₈, R₂₉ and R₃₀ together may possess the atoms necessary to constitute an aromatic ring system, n is an integer ranging from 1 to 4, and A is a physiologically acceptable counter ion;
(CH₂)ₙOPO(OR₃₁)₂ and (CH₂)ₙPO(OR₃₁)₂, where R₃₁ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and n is an integer ranging from 0 to 4;
(CH₂)ₙOH and (CH₂)ₙOR₃₂, where R₃₂ is selected from alkyl, haloalkyl, heteroalkyl, haloheteroalkyl, aryl, heteroaryl, heterocycle, a protecting group, a mono-, di- or polyhydroxyalkyl residue, a mono-, di-, or polyhydroxyaryl residue, and a functional group of less than about 100,000 daltons, and n is an integer ranging from 0 to 4;
(CH₂)ₙNHCOR₃₃ and (CH₂)ₙNHNHCOR₃₃, where R₃₃ is selected from a straight or branched chain C1-C20 alkyl, OH, haloalkyl, heteroalkyl, haloheteroalkyl, aryl, heteroaryl, heterocycle, and a functional group of less than about 100,000 daltons, and n is an integer ranging from 1 to 4;
(CH₂)ₙSO₂NHR₃₄, (CH₂)ₙSO₂N(R₃₄)₂, (CH₂)ₙSO₂NHNHR₃₄, (CH₂)ₙSO₂R₃₄, (CH₂)ₙOSO₂NHR₃₄, (CH₂)ₙOSO₂N(R₃₄)₂, (CH₂)ₙOSO₂NHNHR₃₄, and (CH₂)ₙOSO₃R₃₄, where R₃₄ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, NHR₃₄ can constitute an amino acid, an amino acid salt, an amino acid ester residue, or an amino acid amide residue, and n is an integer ranging from 1 to 4; and
aryl or substituted aryl, which may bear one or more substituents with a molecular weight of less than or equal to about 100,000 daltons; and wherein
M is selected from 2H, a metal cation, and photoactive metal ions selected from Ga³⁺; Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

23. A pharmaceutical composition comprising an effective diagnostic or therapeutic amount of the compound of claim 22, together with at least one pharmaceutically acceptable carrier or excipient.

24. The pharmaceutical composition according to claim 23 used to treat ophthalmic diseases.

25. The pharmaceutical composition of claim 24, wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

26. The pharmaceutical composition according to claim 23 used to treat cancer or malignant diseases.

27. The pharmaceutical composition according to claim 23 used to treat cardiovascular diseases.

28. The pharmaceutical composition according to claim 23 used to treat skin diseases.

29. Compounds of formula I: 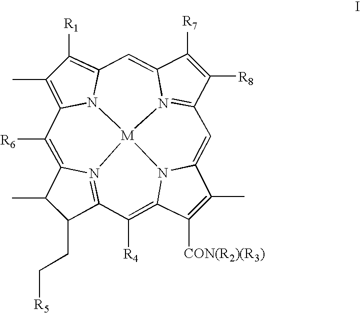
wherein:
R₁, R₇, and R₈ can be the same or different and are selected from vinyl, hydroxyalkyl, alkylether, and CH₂CH₂OCOCH₃;
R₆ is selected from methyl, halogen, CH═CHCHO, CH═CHCH₂OH, CH═CHCH₂O-alkyl, and CHCH₂O-alkylether;
R₄ is selected from:
(CH₂)ₙCO₂R₉, (CHX)ₙCO₂R₉, and (CX₂)ₙCO₂R₉, where X is selected from OH, OR₁₀, SR₁₀, and a halogen, and R₉ and R₁₀ can be the same or different and are selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons, and n is an integer ranging from 1 to 4;
(CH₂)ₙCON(R₁₁)₂, (CH₂)ₙCOR₁₁, (CH₂)ₙCON(R₁₁)(R₁₂), (CX₂)ₙCONH(R₁₁), (CX₂)ₙCON(R₁₂)₂, (CX₂)ₙCON(R₁₁)(R₁₂), (CX₂)ₙCOR₁₁, (CH₂)ₙCONHNH(R₁₁), (CX₂)ₙCONHNH(R₁₁), (CHX)ₙCONH(R₁₁), (CHX)ₙCONHNH(R₁₁), (CHX)ₙCO(R₁₁), (CHX)ₙCON(R₁₁)₂, and (CHX)ₙCON(R₁₁)(R₁₂), where X is selected from OH, OR₁₃, SR₁₃, and a halogen, and R₁₁, R₁₂ and R₁₃ can be the same or different and are selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
(CH₂)ₙCONH(R₁₁), where R₁₁ is selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
(CH₂)ₙS(R₁₄), (CH₂)ₙNH(R₁₄), (CH₂)ₙNHNH(R₁₄), (CH₂)ₙN(R₁₄)₂, (CH₂)ₙN(R₁₄)(R₁₅), (CH₂)ₙN(R₁₄)(R₁₅)(R₁₆)⁺A, amino acids containing —NH(R₁₄), amino acid esters containing —NH(R₁₄), and amino acid amides containing —NH(R₁₄), where R₁₄, R₁₅ and R₁₆ can be the same or different and are selected from H, OH, NH₂, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, where R₁₄, R₁₅ and R₁₆ together may possess the atoms necessary to constitute an aromatic ring system, n is an integer ranging from 1 to 4, and A is a physiologically acceptable counter ion;
(CH₂)ₙOPO(OR₁₇)₂ and (CH₂)ₙPO(OR₁₇)₂, where R₁₇ is selected from H, OH, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and n is an integer ranging from 0 to 4;
(CH₂)ₙNHCOR₁₈ and (CH₂)ₙNHNHCOR₁₈, where R₁₈ is selected from a straight or branched chain C1-C20 alkyl, haloalkyl, heteroalkyl, haloheteroalkyl, aryl, heteroaryl, heterocycle, and a functional group of less than about 100,000 daltons, and n is an integer ranging from 0 to 4;
(CH₂)ₙSO₂NHR₁₉, (CH₂)ₙSO₂N(R₁₉)₂, (CH₂)ₙSO₂NHNHR₁₉, (CH₂)ₙSO₂R₁₉, (CH₂)ₙOSO₂NHR₁₉, (CH₂)ₙOSO₂N(R₁₉)₂, (CH₂)ₙOSO₂NHNHR₁₉, and (CH₂)ₙOSO₃R₁₉, where R₁₉ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and NHR₁₉ can constitute an amino acid, an amino acid salt, an amino acid ester residue, or an amino acid amide residue, and n is an integer between 0 and 4;
(CH₂)ₙOH and (CH₂)ₙOR₂₀, where R₂₀ is selected from alkyl, haloalkyl, heteroalkyl, haloheteroalkyl, aryl, heteroaryl, heterocycle, a protecting group, a mono-, di- or polyhydroxyalkyl residue, a mono-, di-, or polyhydroxyaryl residue, and a functional group of less than about 100,000 daltons, and n is an integer ranging from 0 to 4; and
aryl or substituted aryl, which may bear one or more substituents with a molecular weight of less than or equal to about 100,000 daltons;
R₂ and R₃ can be the same or different and are selected from alkyl, cycloalkyl, aryl, (CH₂)_{w}OH , (CH₂)ₙO-alkyl, (CH₂)ₙOCOCH₃, CH₂CH(OH)CH₂OH, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, ((CH₂)ₙOn)ₘ(CH₂)_{Q}OH, ((CH₂)ₙO)ₘ(CH₂O)_{Q}COCH₃, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN((CH₂)ₘN(CH₃)₂)₂, (CH₂)ₙN((CH₂)ₘN(CH₃)₃⁺A)₂, ((CH₂)ₙO)ₘ(CH₂)_{Q}OH, ((CH₂)ₙO)ₘ(CH₂O)_{Q}COCH₃, (CH₂)ₙO-haloalkyl, (CH₂)ₙN((CH₂)ₘO-alkyl)₂, (CH₂)ₙN((CH₂)ₘO-alkylether)₂, a mono-, di-, or polyhydroxyalkyl residue, CH₂CH(OAc)CH₂OAc, an alkylphosphate residue, an alkylsulfonic acid residue, an alkylsulfonic ester or amide residue, an alkylmorpholine residue, an alkylheterocyclic residue, an alkylthiol residue, a mono-, di-, or polyhydroxyaryl residue, a mono-, di-, or polyetheralkyl residue, and a mono-, di-, or polyetheraryl residue, wherein Q, n, and m may be the same or different and are integers ranging from 0 to 10,000, w is an integer ranging from 1 to 10,000, and A is a charge balancing ion; and
M is selected from 2H, a metal cation, or photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

30. A pharmaceutical composition comprising an effective diagnostic or therapeutic amount of the compound of claim 29, together with at least one pharmaceutically acceptable carrier or excipient.

31. The pharmaceutical composition according to claim 30 used to treat ophthalmic diseases.

32. The pharmaceutical composition of claim 31, wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

33. The pharmaceutical composition according to claim 30 used to treat cancer or malignant diseases.

34. The pharmaceutical composition according to claim 30 used to treat cardiovascular diseases.

35. The pharmaceutical composition according to claim 30 used to treat skin diseases.
